(19) United States
(12) Patent Application Publication
(10) Pub. No.: US 2023/0029970 A1
(43) Pub. Date: Feb. 2, 2023

(54) QUALITY SCORE CALIBRATION OF BASECALLING SYSTEMS

(71) Applicants: ILLUMINA, INC., San Diego, CA (US); ILLUMINA SOFTWARE, INC., San Diego, CA (US)

(72) Inventors: Rohan PAUL, Palo Alto, CA (US); Dorna KASHEFHAGHIGHI, San Francisco, CA (US); John S. VIECELI, Encinitas, CA (US); Andrew Dodge HEIBERG, San Diego, CA (US)

(73) Assignees: ILLUMINA, INC., San Diego, CA (US); ILLUMINA SOFTWARE, INC., San Diego, CA (US)

(21) Appl. No.: 17/839,387

(22) Filed: Jun. 13, 2022

Related U.S. Application Data
(60) Provisional application No. 63/226,707, filed on Jul. 28, 2021.

(51) Int. Cl. G16B 30/00; C12Q 1/6869

(52) U.S. Cl. CPC G16B 30/00; C12Q 1/6869

(57) ABSTRACT

A method of generating base calls by a base caller is disclosed. The method includes receiving a plurality of sensor data from a flow cell, wherein the plurality of sensor data is within a first range and identifying a second range, such that at least a threshold percentage of the plurality of sensor data are within the second range. At least a subset of the plurality of sensor data, that are within the second range, are mapped to a third range, thereby generating a plurality of normalized sensor data. The plurality of normalized sensor data is processed in a base caller, to call, for the plurality of normalized sensor data, one or more corresponding bases.

### PRIORITY APPLICATION

This application claims the benefit of U.S. Provisional Patent Application No. 63/226,707, titled, “Quality Score Calibration of Basecalling Systems,” filed Jul. 28, 2021 (Attorney Docket No. ILLM 1045-1/IP-2093-PRV). The provisional application is hereby incorporated by reference for all purposes.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep neural networks such as deep convolutional neural networks for analyzing data.

### INCORPORATIONS

The following are incorporated by reference as if fully set forth herein:

U.S. Provisional Patent Application No. 62/979,384, titled “ARTIFICIAL INTELLIGENCE-BASED BASE CALLING OF INDEX SEQUENCES,” filed 20 Feb. 2020 (Attorney Docket No. ILLM 1015-1/IP-1857-PRV);

U.S. Provisional Patent Application No. 62/979,414, titled “ARTIFICIAL INTELLIGENCE-BASED MANY-TO-MANY BASE CALLING,” filed 20 Feb. 2020 (Attorney Docket No. ILLM 1016-1/IP-1858-PRV);

U.S. Nonprovisional patent application Ser. No. 16/825,987, titled “TRAINING DATA GENERATION FOR ARTIFICIAL INTELLIGENCE-BASED SEQUENCING,” filed 20 Mar. 2020 (Attorney Docket No. ILLM 1008-16/IP-1693-US);

U.S. Nonprovisional patent application Ser. No. 16/825,991, titled “ARTIFICIAL INTELLIGENCE-BASED GENERATION OF SEQUENCING METADATA,” filed 20 Mar. 2020 (Attorney Docket No. ILLM 1008-17/IP-1741-US);

U.S. Nonprovisional patent application Ser. No. 16/826,126, titled “ARTIFICIAL INTELLIGENCE-BASED BASE CALLING,” filed 20 Mar. 2020 (Attorney Docket No. ILLM 1008-18/IP-1744-US);

U.S. Nonprovisional patent application Ser. No. 16/826,134, titled “ARTIFICIAL INTELLIGENCE-BASED SEQUENCING,” filed 21 Mar. 2020 (Attorney Docket No. ILLM 1008-20/IP-752-PRV-US).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

The rapid improvement in computation capability has made deep Convolution Neural Networks (CNNs) a great success in recent years on many computer vision tasks with significantly improved accuracy. During the inference phase, many applications demand low latency processing of one image with strict power consumption requirements, which reduces the efficiency of Graphics Processing Unit (GPU) and other general-purpose platforms, bringing opportunities for specific acceleration hardware, e.g., Field Programmable Gate Array (FPGA), by customizing the digital circuit specific for the deep learning algorithm inference. However, deploying CNNs on portable and embedded systems is still challenging due to large data volume, intensive computation, varying algorithm structures, and frequent memory accesses.

As convolution contributes to most operations in CNNs, the convolution acceleration scheme significantly affects the efficiency and performance of a hardware CNN accelerator. Convolution involves multiply and accumulate (MAC) operations with four levels of loops that slide along kernel and feature maps. The first loop level computes the MAC of pixels within a kernel window. The second loop level accumulates the sum of products of the MAC across different input feature maps. After finishing the first and second loop levels, a final output element in the output feature map is obtained by adding the bias. The third loop level slides the kernel window within an input feature map. The fourth loop level generates different output feature maps.

FPGAs have gained increasing interest and popularity in particular to accelerate inference tasks, due to their (1) high degree of reconfigurability, (2) faster development time compared to Application Specific Integrated Circuits (ASICs) to catch up with the rapid evolution of CNNs, (3) good performance, and (4) superior energy efficiency compared to GPUs. The high performance and efficiency of an FPGA can be realized by synthesizing a circuit that is customized for a specific computation to directly process billions of operations with the customized memory systems. For instance, hundreds to thousands of digital signal processing (DSP) blocks on modern FPGAs support the core convolution operation, e.g., multiplication and addition, with high parallelism. Dedicated data buffers between external on-chip memory and on-chip processing engines (PEs) can be designed to realize the preferred dataflow by configuring tens of Mbyte on-chip block random access memories (BRAM) on the FPGA chip.

Efficient dataflow and hardware architecture of CNN acceleration are desired to minimize data communication while maximizing resource utilization to achieve high performance. An opportunity arises to design methodology and framework to accelerate the inference process of various CNN algorithms on acceleration hardware with high performance, efficiency, and flexibility.

Deep neural networks have great promise for bioinformatics research because of their broad applicability and enhanced prediction power. Convolutional neural networks have been adapted to solve sequence-based problems in genomics such as motif discovery, pathogenic variant identification, and gene expression inference. Convolutional neural networks use a weight-sharing strategy that is especially useful for studying DNA because it can capture sequence motifs, which are short, recurring local patterns in DNA that are presumed to have significant biological functions. Neural networks can capture long-range dependencies in sequential data of varying lengths, such as protein or DNA sequences. Therefore, an opportunity arises to use a principled deep learning-based framework for base calling.

There is a need for increasing the quality and quantity of nucleic acid sequencing data that can be obtained rapidly and cost-effectively for a wide variety of uses, including for genomics (e.g., for genome characterization of any and all animal, plant, microbial or other biological species or populations), pharmacogenomics, transcriptomics, diagnostics, prognostics, biomedical risk assessment, clinical and research genetics, personalized medicine, drug efficacy and drug interactions assessments, veterinary medicine, agriculture, evolutionary and biodiversity studies, aquaculture, forestry, oceanography, ecological and environmental management, and other purposes. For example, deep learning network models or other appropriate models may be used to generate sequencing data for a wide variety of genomics.

Such models, in addition to generating base calls, also generate corresponding quality scores. Generally speaking, quality scores provide indication of, in logarithmic scale, probabilities of a base being called an adenine (A), thymine (T), guanine (G), or cytosine (C). For example, a quality score Q(A) for a base provides an indication of a probability of the base being an A; a quality score Q(C) for the base provides an indication of a probability of the base being an C, and so on.

Often times, the quality scores are used to make critical decisions, such as critical health care decisions. For example, in a healthcare setting, quality scores associated with detecting bases of a human tissue sample may affect an approach to treat a health condition. Thus, it is desirable that the quality scores generated for base calling are relatively accurate and dependable. For example, it is desirable that the quality scores generated for base calling are better aligned to empirically determined quality scores (which are representative of true quality scores).

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 illustrates a cross-section of a biosensor that can be used in various embodiments.

FIG. 2 depicts one implementation of a flow cell that contains clusters in its tiles.

FIG. 3 illustrates an example flow cell with eight lanes, and also illustrates a zoom-in on one tile and its clusters and their surrounding background.

FIG. 4 is a simplified block diagram of the system for analysis of sensor data from a sequencing system, such as base call sensor outputs.

FIG. 5 is a simplified diagram showing aspects of the base calling operation, including functions of a runtime program executed by a host processor.

FIG. 6 is a simplified diagram of a configuration of a configurable processor such as that of FIG. 4.

FIG. 7 is a diagram of a neural network architecture which can be executed using a configurable or a reconfigurable array configured as described herein.

FIG. 8A is a simplified illustration of an organization of tiles of sensor data used by a neural network architecture like that of FIG. 7.

FIG. 8B is a simplified illustration of patches of tiles of sensor data used by a neural network architecture like that of FIG. 7.

FIG. 9 illustrates part of a configuration for a neural network like that of FIG. 7 on a configurable or a reconfigurable array, such as a Field Programmable Gate Array (FPGA).

FIG. 10 is a diagram of another alternative neural network architecture which can be executed using a configurable or a reconfigurable array configured as described herein.

FIG. 11 illustrates one implementation of a specialized architecture of the neural network-based base caller that is used to segregate processing of data for different sequencing cycles.

FIG. 12 depicts one implementation of segregated layers, each of which can include convolutions.

FIG. 13A depicts one implementation of combinatory layers, each of which can include convolutions.

FIG. 13B depicts another implementation of the combinatory layers, each of which can include convolutions.

FIG. 14A illustrates a base calling system generating quality scores corresponding to A, C, T, and G for various bases to be called.

FIG. 14B illustrates a table indicating a relationship between probability scores, quality scores, corresponding error probabilities, and corresponding error rates.

FIG. 14C illustrates a comparison operation between predicted quality scores predicted by the base calling system of FIG. 14A and true (e.g., empirically calculated) quality scores.

FIG. 14D illustrates determination of true (e.g., empirically determined) quality scores of FIG. 14C.

FIG. 15A illustrates a graph depicting a comparison between predicted quality scores and true quality scores.

FIG. 15B illustrates another graph depicting another comparison between predicted quality scores and true quality scores.

FIG. 16 illustrates another graph depicting a comparison between predicted quality scores and true quality scores.

FIG. 17A illustrates a base calling system including a normalization module for normalizing sensor data that are received by a base caller.

FIG. 17B illustrates two graphs depicting a normalization operation on sensor data performed by the normalization module of the base calling system of FIG. 17A.

FIG. 17C illustrates a graph depicting a comparison between predicted quality scores and true quality scores, wherein the sensor data have been normalized by the normalization module of the base calling system of FIG. 17A while generating data for the graph of FIG. 17C.

FIG. 17D illustrates a plot indicating expected calibration error (ECE) for a base calling system having input normalization versus another base calling system lacking such an input normalization.

FIG. 17E illustrates a color comparison between sensor data prior to normalization and normalized sensor data.

FIG. 17F illustrates a flowchart depicting an example method for normalizing sensor data, and using normalized sensor data for base calling operations.

FIG. 18A illustrates a base calling system including a quality score remapping module for selectively remapping quality scores predicted by the base caller of the base calling system.

FIGS. 18B1, 18B2, 18B3, 18B4, and 18B5, in combination, illustrate examples of quality score remapping and quantization.

FIG. 18C illustrates two further examples of quality score remapping and quantization.

FIG. 19 illustrates a table depicting, for some specific base sequences, deviations between (i) an average of quality scores of the specific base sequences and (ii) an average of remapped quality scores of the specific base sequences, where the remapping is performed in accordance with a general Look Up Table (LUT) of, for example, FIG. 18B2.

FIG. 20A illustrates a LUT that is usable to remap predicted quality scores of a specific base sequence to remapped quality scores.

FIG. 20B illustrates remapping of predicted quality scores for a specific base sequence using the LUT of FIG. 20A.

FIG. 21 illustrates a base calling system that includes a loss penalization module to selectively penalize loss for one or more specific base sequences.

FIGS. 22A, 22B, 22C, 22D and 22E, in combination, illustrate penalization of a loss function (e.g., by the loss penalization module 2106), in response to a detection of a specific base sequence.

FIG. 22F illustrates application of a specialized weight to loss associated with a middle base of a specific base sequence.

FIG. 22G illustrates two graphs comparing performance of a base calling system that does not penalize loss, versus a base calling system that penalizes loss for a specific base sequence.

FIG. 23 illustrates a base calling system that includes (i) the normalization module of the base calling system of FIG. 17A, (ii) the quality score remapping module and the quality score quantization module of the base calling system of FIG. 18A, and (iii) the loss penalization module of the base calling system of FIG. 21.

FIG. 24 is a block diagram of a base calling system in accordance with one implementation.

FIG. 25 is a block diagram of a system controller that can be used in the system of FIG. 24.

FIG. 26 is a simplified block diagram of a computer system that can be used to implement the technology disclosed.

### DETAILED DESCRIPTION

As used herein, the terms “polynucleotide” or “nucleic acids” refer to deoxyribonucleic acid (DNA), but where appropriate the skilled artisan will recognize that the systems and devices herein can also be utilized with ribonucleic acid (RNA). The terms should be understood to include, as equivalents, analogs of either DNA or RNA made from nucleotide analogs. The terms as used herein also encompass cDNA, that is complementary, or copy, DNA produced from an RNA template, for example by the action of reverse transcriptase.

The single stranded polynucleotide molecules sequenced by the systems and devices herein can have originated in single-stranded form, as DNA or RNA or have originated in double-stranded DNA (dsDNA) form (e.g., genomic DNA fragments, PCR and amplification products and the like). Thus, a single stranded polynucleotide may be the sense or antisense strand of a polynucleotide duplex. Methods of preparation of single stranded polynucleotide molecules suitable for use in the method of the disclosure using standard techniques are well known in the art. The precise sequence of the primary polynucleotide molecules is generally not material to the disclosure, and may be known or unknown. The single stranded polynucleotide molecules can represent genomic DNA molecules (e.g., human genomic DNA) including both intron and exon sequences (coding sequences), as well as non-coding regulatory sequences such as promoter and enhancer sequences.

In certain embodiments, the nucleic acid to be sequenced through use of the current disclosure is immobilized upon a substrate (e.g., a substrate within a flowcell or one or more beads upon a substrate such as a flowcell, etc.). The term “immobilized” as used herein is intended to encompass direct or indirect, covalent or non-covalent attachment, unless indicated otherwise, either explicitly or by context. In certain embodiments covalent attachment may be preferred, but generally all that is required is that the molecules (e.g. nucleic acids) remain immobilized or attached to the support under conditions in which it is intended to use the support, for example in applications requiring nucleic acid sequencing.

The term “solid support” (or “substrate” in certain usages) as used herein refers to any inert substrate or matrix to which nucleic acids can be attached, such as for example glass surfaces, plastic surfaces, latex, dextran, polystyrene surfaces, polypropylene surfaces, polyacrylamide gels, gold surfaces, and silicon wafers. In many embodiments, the solid support is a glass surface (e.g., the planar surface of a flowcell channel). In certain embodiments the solid support may comprise an inert substrate or matrix which has been “functionalized,” for example by the application of a layer or coating of an intermediate material comprising reactive groups which permit covalent attachment to molecules such as polynucleotides. By way of non-limiting example such supports can include polyacrylamide hydrogels supported on an inert substrate such as glass. In such embodiments the molecules (polynucleotides) can be directly covalently attached to the intermediate material (e.g., the hydrogel) but the intermediate material can itself be non-covalently attached to the substrate or matrix (e.g., the glass substrate). Covalent attachment to a solid support is to be interpreted accordingly as encompassing this type of arrangement.

As indicated above, the present disclosure comprises novel systems and devices for sequencing nucleic acids. As will be apparent to those of skill in the art, references herein to a particular nucleic acid sequence may, depending on the context, also refer to nucleic acid molecules which comprise such nucleic acid sequence. Sequencing of a target fragment means that a read of the chronological order of bases is established. The bases that are read do not need to be contiguous, although this is preferred, nor does every base on the entire fragment have to be sequenced during the sequencing. Sequencing can be carried out using any suitable sequencing technique, wherein nucleotides or oligonucleotides are added successively to a free 3′ hydroxyl group, resulting in synthesis of a polynucleotide chain in the 5′ to 3′ direction. The nature of the nucleotide added is preferably determined after each nucleotide addition. Sequencing techniques using sequencing by ligation, wherein not every contiguous base is sequenced, and techniques such as massively parallel signature sequencing (MPSS) where bases are removed from, rather than added to, the strands on the surface are also amenable to use with the systems and devices of the disclosure.

In certain embodiments, the current disclosure discloses sequencing-by-synthesis (SBS). In SBS, four fluorescently labeled modified nucleotides are used to sequence dense clusters of amplified DNA (possibly millions of clusters) present on the surface of a substrate (e.g., a flowcell). Various additional aspects regarding SBS procedures and methods, which can be utilized with the systems and devices herein, are disclosed in, for example, WO04018497, WO04018493 and U.S. Pat. No. 7,057,026 (nucleotides), WO05024010 and WO06120433 (polymerases), WO05065814 (surface attachment techniques), and WO 9844151, WO06064199 and WO07010251, the contents of each of which are incorporated herein by reference in their entirety.

In particular uses of the systems/devices herein, the flowcells containing the nucleic acid samples for sequencing are placed within the appropriate flowcell holder. The samples for sequencing can take the form of single molecules, amplified single molecules in the form of clusters, or beads comprising molecules of nucleic acid. The nucleic acids are prepared such that they comprise an oligonucleotide primer adjacent to an unknown target sequence. To initiate the first SBS sequencing cycle, one or more differently labeled nucleotides, and DNA polymerase, etc., are flowed into/through the flowcell by the fluid flow subsystem (various embodiments of which are described herein). Either a single nucleotide can be added at a time, or the nucleotides used in the sequencing procedure can be specially designed to possess a reversible termination property, thus allowing each cycle of the sequencing reaction to occur simultaneously in the presence of all four labeled nucleotides (A, C, T, G). Where the four nucleotides are mixed together, the polymerase is able to select the correct base to incorporate and each sequence is extended by a single base. In such methods of using the systems, the natural competition between all four alternatives leads to higher accuracy than wherein only one nucleotide is present in the reaction mixture (where most of the sequences are therefore not exposed to the correct nucleotide). Sequences where a particular base is repeated one after another (e.g., homopolymers) are addressed like any other sequence and with high accuracy.

The fluid flow subsystem also flows the appropriate reagents to remove the blocked 3′ terminus (if appropriate) and the fluorophore from each incorporated base. The substrate can be exposed either to a second round of the four blocked nucleotides, or optionally to a second round with a different individual nucleotide. Such cycles are then repeated, and the sequence of each cluster is read over the multiple chemistry cycles. The computer aspect of the current disclosure can optionally align the sequence data gathered from each single molecule, cluster or bead to determine the sequence of longer polymers, etc. Alternatively, the image processing and alignment can be performed on a separate computer.

The heating/cooling components of the system regulate the reaction conditions within the flowcell channels and reagent storage areas/containers (and optionally the camera, optics, and/or other components), while the fluid flow components allow the substrate surface to be exposed to suitable reagents for incorporation (e.g., the appropriate fluorescently labeled nucleotides to be incorporated) while unincorporated reagents are rinsed away. An optional movable stage upon which the flowcell is placed allows the flowcell to be brought into proper orientation for laser (or other light) excitation of the substrate and optionally moved in relation to a lens objective to allow reading of different areas of the substrate. Additionally, other components of the system are also optionally movable/adjustable (e.g., the camera, the lens objective, the heater/cooler, etc.). During laser excitation, the image/location of emitted fluorescence from the nucleic acids on the substrate is captured by the camera component, thereby, recording the identity, in the computer component, of the first base for each single molecule, cluster or bead.

Embodiments described herein may be used in various biological or chemical processes and systems for academic or commercial analysis. More specifically, embodiments described herein may be used in various processes and systems where it is desired to detect an event, property, quality, or characteristic that is indicative of a desired reaction. For example, embodiments described herein include cartridges, biosensors, and their components as well as bioassay systems that operate with cartridges and biosensors. In particular embodiments, the cartridges and biosensors include a flow cell and one or more sensors, pixels, light detectors, or photodiodes that are coupled together in a substantially unitary structure.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be standalone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” or “including” an element or a plurality of elements having a particular property may include additional elements whether or not they have that property.

As used herein, a “desired reaction” includes a change in at least one of a chemical, electrical, physical, or optical property (or quality) of an analyte-of-interest. In particular embodiments, the desired reaction is a positive binding event (e.g., incorporation of a fluorescently labeled biomolecule with the analyte-of-interest). More generally, the desired reaction may be a chemical transformation, chemical change, or chemical interaction. The desired reaction may also be a change in electrical properties. For example, the desired reaction may be a change in ion concentration within a solution. Exemplary reactions include, but are not limited to, chemical reactions such as reduction, oxidation, addition, elimination, rearrangement, esterification, amidation, etherification, cyclization, or substitution; binding interactions in which a first chemical binds to a second chemical; dissociation reactions in which two or more chemicals detach from each other; fluorescence; luminescence; bioluminescence; chemiluminescence; and biological reactions, such as nucleic acid replication, nucleic acid amplification, nucleic acid hybridization, nucleic acid ligation, phosphorylation, enzymatic catalysis, receptor binding, or ligand binding. The desired reaction can also be an addition or elimination of a proton, for example, detectable as a change in pH of a surrounding solution or environment. An additional desired reaction can be detecting the flow of ions across a membrane (e.g., natural or synthetic bilayer membrane), for example as ions flow through a membrane the current is disrupted and the disruption can be detected.

In particular embodiments, the desired reaction includes the incorporation of a fluorescently-labeled molecule to an analyte. The analyte may be an oligonucleotide and the fluorescently-labeled molecule may be a nucleotide. The desired reaction may be detected when an excitation light is directed toward the oligonucleotide having the labeled nucleotide, and the fluorophore emits a detectable fluorescent signal. In alternative embodiments, the detected fluorescence is a result of chemiluminescence or bioluminescence. A desired reaction may also increase fluorescence (or Forster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

As used herein, a “reaction component” or “reactant” includes any substance that may be used to obtain a desired reaction. For example, reaction components include reagents, enzymes, samples, other biomolecules, and buffer solutions. The reaction components are typically delivered to a reaction site in a solution and/or immobilized at a reaction site. The reaction components may interact directly or indirectly with another substance, such as the analyte-of-interest.

As used herein, the term “reaction site” is a localized region where a desired reaction may occur. A reaction site may include support surfaces of a substrate where a substance may be immobilized thereon. For example, a reaction site may include a substantially planar surface in a channel of a flow cell that has a colony of nucleic acids thereon. Typically, but not always, the nucleic acids in the colony have the same sequence, being for example, clonal copies of a single stranded or double stranded template. However, in some embodiments a reaction site may contain only a single nucleic acid molecule, for example, in a single stranded or double stranded form. Furthermore, a plurality of reaction sites may be unevenly distributed along the support surface or arranged in a predetermined manner (e.g., side-by-side in a matrix, such as in microarrays). A reaction site can also include a reaction chamber (or well) that at least partially defines a spatial region or volume configured to compartmentalize the desired reaction.

This application uses the terms “reaction chamber” and “well” interchangeably. As used herein, the term “reaction chamber” or “well” includes a spatial region that is in fluid communication with a flow channel. The reaction chamber may be at least partially separated from the surrounding environment or other spatial regions. For example, a plurality of reaction chambers may be separated from each other by shared walls. As a more specific example, the reaction chamber may include a cavity defined by interior surfaces of a well and have an opening or aperture so that the cavity may be in fluid communication with a flow channel. Biosensors including such reaction chambers are described in greater detail in international application no. PCT/US2011/057111, filed on Oct. 20, 2011, which is incorporated herein by reference in its entirety.

In some embodiments, the reaction chambers are sized and shaped relative to solids (including semi-solids) so that the solids may be inserted, fully or partially, therein. For example, the reaction chamber may be sized and shaped to accommodate only one capture bead. The capture bead may have clonally amplified DNA or other substances thereon. Alternatively, the reaction chamber may be sized and shaped to receive an approximate number of beads or solid substrates. As another example, the reaction chambers may also be filled with a porous gel or substance that is configured to control diffusion or filter fluids that may flow into the reaction chamber.

In some embodiments, sensors (e.g., light detectors, photodiodes) are associated with corresponding pixel areas of a sample surface of a biosensor. As such, a pixel area is a geometrical construct that represents an area on the biosensor's sample surface for one sensor (or pixel). A sensor that is associated with a pixel area detects light emissions gathered from the associated pixel area when a desired reaction has occurred at a reaction site or a reaction chamber overlying the associated pixel area. In a flat surface embodiment, the pixel areas can overlap. In some cases, a plurality of sensors may be associated with a single reaction site or a single reaction chamber. In other cases, a single sensor may be associated with a group of reaction sites or a group of reaction chambers.

As used herein, a “biosensor” includes a structure having a plurality of reaction sites and/or reaction chambers (or wells). A biosensor may include a solid-state imaging device (e.g., CCD or CMOS imager) and, optionally, a flow cell mounted thereto. The flow cell may include at least one flow channel that is in fluid communication with the reaction sites and/or the reaction chambers. As one specific example, the biosensor is configured to fluidically and electrically couple to a bioassay system. The bioassay system may deliver reactants to the reaction sites and/or the reaction chambers according to a predetermined protocol (e.g., sequencing-by-synthesis) and perform a plurality of imaging events. For example, the bioassay system may direct solutions to flow along the reaction sites and/or the reaction chambers. At least one of the solutions may include four types of nucleotides having the same or different fluorescent labels. The nucleotides may bind to corresponding oligonucleotides located at the reaction sites and/or the reaction chambers. The bioassay system may then illuminate the reaction sites and/or the reaction chambers using an excitation light source (e.g., solid-state light sources, such as light-emitting diodes or LEDs). The excitation light may have a predetermined wavelength or wavelengths, including a range of wavelengths. The excited fluorescent labels provide emission signals that may be captured by the sensors.

In alternative embodiments, the biosensor may include electrodes or other types of sensors configured to detect other identifiable properties. For example, the sensors may be configured to detect a change in ion concentration. In another example, the sensors may be configured to detect the ion current flow across a membrane.

As used herein, a “cluster” is a colony of similar or identical molecules or nucleotide sequences or DNA strands. For example, a cluster can be an amplified oligonucleotide or any other group of a polynucleotide or polypeptide with a same or similar sequence. In other embodiments, a cluster can be any element or group of elements that occupy a physical area on a sample surface. In embodiments, clusters are immobilized to a reaction site and/or a reaction chamber during a base calling cycle.

As used herein, the term “immobilized,” when used with respect to a biomolecule or biological or chemical substance, includes substantially attaching the biomolecule or biological or chemical substance at a molecular level to a surface. For example, a biomolecule or biological or chemical substance may be immobilized to a surface of the substrate material using adsorption techniques including non-covalent interactions (e.g., electrostatic forces, van der Waals, and dehydration of hydrophobic interfaces) and covalent binding techniques where functional groups or linkers facilitate attaching the biomolecules to the surface Immobilizing biomolecules or biological or chemical substances to a surface of a substrate material may be based upon the properties of the substrate surface, the liquid medium carrying the biomolecule or biological or chemical substance, and the properties of the biomolecules or biological or chemical substances themselves. In some cases, a substrate surface may be functionalized (e.g., chemically or physically modified) to facilitate immobilizing the biomolecules (or biological or chemical substances) to the substrate surface. The substrate surface may be first modified to have functional groups bound to the surface. The functional groups may then bind to biomolecules or biological or chemical substances to immobilize them thereon. A substance can be immobilized to a surface via a gel, for example, as described in US Patent Publ. No. US 2011/0059865 A1, which is incorporated herein by reference.

In some embodiments, nucleic acids can be attached to a surface and amplified using bridge amplification. Useful bridge amplification methods are described, for example, in U.S. Pat. No. 5,641,658; WO 2007/010251; U.S. Pat. No. 6,090,592; U.S. Patent Publ. No. 2002/0055100 A1; U.S. Pat. No. 7,115,400; U.S. Patent Publ. No. 2004/0096853 A1; U.S. Patent Publ. No. 2004/0002090 A1; U.S. Patent Publ. No. 2007/0128624 A1; and U.S. Patent Publ. No. 2008/0009420 A1, each of which is incorporated herein in its entirety. Another useful method for amplifying nucleic acids on a surface is Rolling Circle Amplification (RCA), for example, using methods set forth in further detail below. In some embodiments, the nucleic acids can be attached to a surface and amplified using one or more primer pairs. For example, one of the primers can be in solution and the other primer can be immobilized on the surface (e.g., 5′-attached). By way of example, a nucleic acid molecule can hybridize to one of the primers on the surface followed by extension of the immobilized primer to produce a first copy of the nucleic acid. The primer in solution then hybridizes to the first copy of the nucleic acid which can be extended using the first copy of the nucleic acid as a template. Optionally, after the first copy of the nucleic acid is produced, the original nucleic acid molecule can hybridize to a second immobilized primer on the surface and can be extended at the same time or after the primer in solution is extended. In any embodiment, repeated rounds of extension (e.g., amplification) using the immobilized primer and primer in solution provide multiple copies of the nucleic acid.

In particular embodiments, the assay protocols executed by the systems and methods described herein include the use of natural nucleotides and also enzymes that are configured to interact with the natural nucleotides. Natural nucleotides include, for example, ribonucleotides (RNA) or deoxyribonucleotides (DNA). Natural nucleotides can be in the mono-, di-, or tri-phosphate form and can have a base selected from adenine (A), thymine (T), uracil (U), guanine (G) or cytosine (C). It will be understood however that non-natural nucleotides, modified nucleotides or analogs of the aforementioned nucleotides can be used. Some examples of useful non-natural nucleotides are set forth below in regard to reversible terminator-based sequencing by synthesis methods.

In embodiments that include reaction chambers, items or solid substances (including semi-solid substances) may be disposed within the reaction chambers. When disposed, the item or solid may be physically held or immobilized within the reaction chamber through an interference fit, adhesion, or entrapment. Exemplary items or solids that may be disposed within the reaction chambers include polymer beads, pellets, agarose gel, powders, quantum dots, or other solids that may be compressed and/or held within the reaction chamber. In particular embodiments, a nucleic acid superstructure, such as a DNA ball, can be disposed in or at a reaction chamber, for example, by attachment to an interior surface of the reaction chamber or by residence in a liquid within the reaction chamber. A DNA ball or other nucleic acid superstructure can be preformed and then disposed in or at the reaction chamber. Alternatively, a DNA ball can be synthesized in situ at the reaction chamber. A DNA ball can be synthesized by rolling circle amplification to produce a concatemer of a particular nucleic acid sequence and the concatemer can be treated with conditions that form a relatively compact ball. DNA balls and methods for their synthesis are described, for example in, U.S. Patent Publication Nos. 2008/0242560 A1 or 2008/0234136 A1, each of which is incorporated herein in its entirety. A substance that is held or disposed in a reaction chamber can be in a solid, liquid, or gaseous state.

As used herein, “base calling” identifies a nucleotide base in a nucleic acid sequence. Base calling refers to the process of determining a base call (A, C, G, T) for every cluster at a specific cycle. As an example, base calling can be performed utilizing four-channel, two-channel or one-channel methods and systems described in the incorporated materials of U.S. Patent Application Publication No. 2013/0079232. In particular embodiments, a base calling cycle is referred to as a “sampling event.” In one dye and two-channel sequencing protocol, a sampling event comprises two illumination stages in time sequence, such that a pixel signal is generated at each stage. The first illumination stage induces illumination from a given cluster indicating nucleotide bases A and T in a AT pixel signal, and the second illumination stage induces illumination from a given cluster indicating nucleotide bases C and T in a CT pixel signal.

The technology disclosed, e.g., the disclosed base callers can be implemented on processors like Central Processing Units (CPUs), Graphics Processing Units (GPUs), Field Programmable Gate Arrays (FPGAs), Coarse-Grained Reconfigurable Architectures (CGRAs), Application-Specific Integrated Circuits (ASICs), Application Specific Instruction-set Processor (ASIP), and Digital Signal Processors (DSPs).

### Biosensor

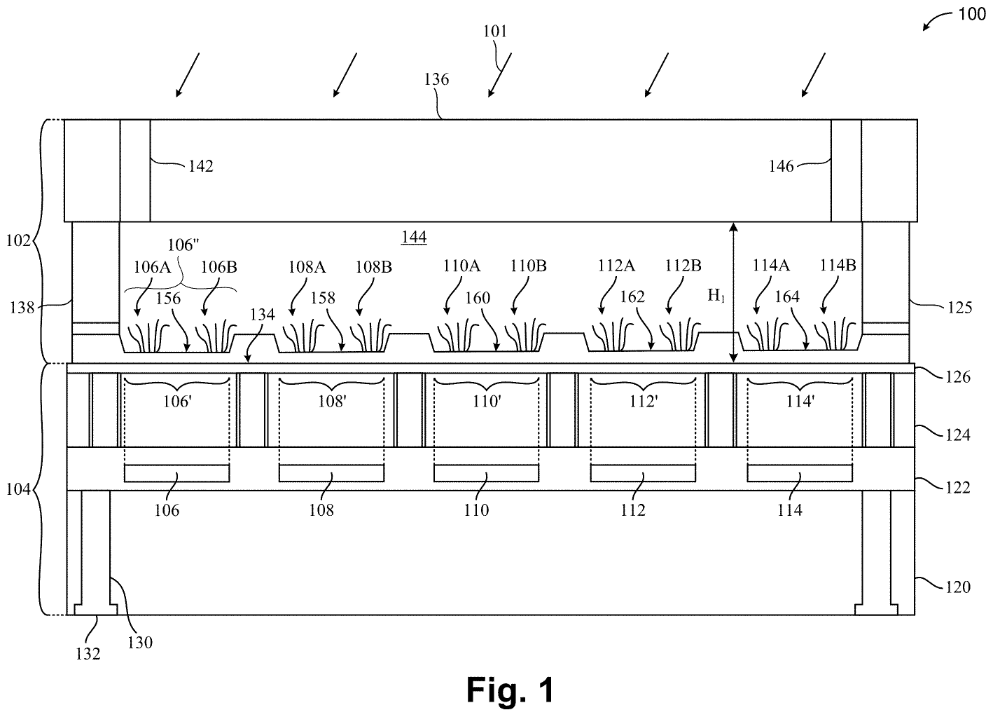

FIG. 1 illustrates a cross-section of a biosensor 100 that can be used in various embodiments. Biosensor 100 has pixel areas 106′, 108′, 110′, 112′, and 114′ that can each hold more than one cluster during a base calling cycle (e.g., 2 clusters per pixel area). As shown, the biosensor 100 may include a flow cell 102 that is mounted onto a sampling device 104. In the illustrated embodiment, the flow cell 102 is affixed directly to the sampling device 104. However, in alternative embodiments, the flow cell 102 may be removably coupled to the sampling device 104. The sampling device 104 has a sample surface 134 that may be functionalized (e.g., chemically or physically modified in a suitable manner for conducting the desired reactions). For example, the sample surface 134 may be functionalized and may include a plurality of pixel areas 106′, 108′, 110′, 112′, and 114′ that can each hold more than one cluster during a base calling cycle (e.g., each having a corresponding cluster pair 106A, 106B; 108A, 108B; 110A, 110B; 112A, 112B; and 114A, 114B immobilized thereto). Each pixel area is associated with a corresponding sensor (or pixel or photodiode) 106, 108, 110, 112, and 114, such that light received by the pixel area is captured by the corresponding sensor. A pixel area 106′ can also be associated with a corresponding reaction site 106″ on the sample surface 134 that holds a cluster pair, such that light emitted from the reaction site 106″ is received by the pixel area 106′ and captured by the corresponding sensor 106. As a result of this sensing structure, in the case in which two or more clusters are present in a pixel area of a particular sensor during a base calling cycle (e.g., each having a corresponding cluster pair), the pixel signal in that base calling cycle carries information based on all of the two or more clusters. As a result, signal processing as described herein is used to distinguish each cluster, where there are more clusters than pixel signals in a given sampling event of a particular base calling cycle.

In the illustrated embodiment, the flow cell 102 includes sidewalls 138, 125, and a flow cover 136 that is supported by the sidewalls 138, 125. The sidewalls 138, 125 are coupled to the sample surface 134, and extend between the flow cover 136 and the sample surface 134. In some embodiments, the sidewalls 138, 125 are formed from a curable adhesive layer that bonds the flow cover 136 to the sampling device 104.

The sidewalls 138, 125 are sized and shaped so that a flow channel 144 exists between the flow cover 136 and the sampling device 104. The flow cover 136 may include a material that is transparent to excitation light 101 propagating from an exterior of the biosensor 100 into the flow channel 144. In an example, the excitation light 101 approaches the flow cover 136 at a non-orthogonal angle.

Also shown, the flow cover 136 may include inlet and outlet ports 142, 146 that are configured to fluidically engage other ports (not shown). For example, the other ports may be from the cartridge or the workstation. The flow channel 144 is sized and shaped to direct a fluid along the sample surface 134. A height Hi and other dimensions of the flow channel 144 may be configured to maintain a substantially even flow of a fluid along the sample surface 134. The dimensions of the flow channel 144 may also be configured to control bubble formation.

By way of example, the flow cover 136 (or the flow cell 102) may comprise a transparent material, such as glass or plastic. The flow cover 136 may constitute a substantially rectangular block having a planar exterior surface and a planar inner surface that defines the flow channel 144. The block may be mounted onto the sidewalls 138, 125. Alternatively, the flow cell 102 may be etched to define the flow cover 136 and the sidewalls 138, 125. For example, a recess may be etched into the transparent material. When the etched material is mounted to the sampling device 104, the recess may become the flow channel 144.

The sampling device 104 may be similar to, for example, an integrated circuit comprising a plurality of stacked substrate layers 120-126. The substrate layers 120-126 may include a base substrate 120, a solid-state imager 122 (e.g., CMOS image sensor), a filter or light-management layer 124, and a passivation layer 126. It should be noted that the above is only illustrative and that other embodiments may include fewer or additional layers. Moreover, each of the substrate layers 120-126 may include a plurality of sub-layers. The sampling device 104 may be manufactured using processes that are similar to those used in manufacturing integrated circuits, such as CMOS image sensors and CCDs. For example, the substrate layers 120-126 or portions thereof may be grown, deposited, etched, and the like to form the sampling device 104.

The passivation layer 126 is configured to shield the filter layer 124 from the fluidic environment of the flow channel 144. In some cases, the passivation layer 126 is also configured to provide a solid surface (i.e., the sample surface 134) that permits biomolecules or other analytes-of-interest to be immobilized thereon. For example, each of the reaction sites may include a cluster of biomolecules that are immobilized to the sample surface 134. Thus, the passivation layer 126 may be formed from a material that permits the reaction sites to be immobilized thereto. The passivation layer 126 may also comprise a material that is at least transparent to a desired fluorescent light. By way of example, the passivation layer 126 may include silicon nitride (Si₂N₄) and/or silica (SiO₂). However, other suitable material(s) may be used. In the illustrated embodiment, the passivation layer 126 may be substantially planar. However, in alternative embodiments, the passivation layer 126 may include recesses, such as pits, wells, grooves, and the like. In the illustrated embodiment, the passivation layer 126 has a thickness that is about 150-200 nm and, more particularly, about 170 nm.

The filter layer 124 may include various features that affect the transmission of light. In some embodiments, the filter layer 124 can perform multiple functions. For instance, the filter layer 124 may be configured to (a) filter unwanted light signals, such as light signals from an excitation light source; (b) direct emission signals from the reaction sites toward corresponding sensors 106, 108, 110, 112, and 114 that are configured to detect the emission signals from the reaction sites; or (c) block or prevent detection of unwanted emission signals from adjacent reaction sites. As such, the filter layer 124 may also be referred to as a light-management layer. In the illustrated embodiment, the filter layer 124 has a thickness that is about 1-5 μm and, more particularly, about 2-4 μm. In alternative embodiments, the filter layer 124 may include an array of microlenses or other optical components. Each of the microlenses may be configured to direct emission signals from an associated reaction site to a sensor.

In some embodiments, the solid-state imager 122 and the base substrate 120 may be provided together as a previously constructed solid-state imaging device (e.g., CMOS chip). For example, the base substrate 120 may be a wafer of silicon and the solid-state imager 122 may be mounted thereon. The solid-state imager 122 includes a layer of semiconductor material (e.g., silicon) and the sensors 106, 108, 110, 112, and 114. In the illustrated embodiment, the sensors are photodiodes configured to detect light. In other embodiments, the sensors comprise light detectors. The solid-state imager 122 may be manufactured as a single chip through a CMOS-based fabrication processes.

The solid-state imager 122 may include a dense array of sensors 106, 108, 110, 112, and 114 that are configured to detect activity indicative of a desired reaction from within or along the flow channel 144. In some embodiments, each sensor has a pixel area (or detection area) that is about 1-2 square micrometers (μm²). The array can include 500,000 sensors, 5 million sensors, 10 million sensors, or even 120 million sensors. The sensors 106, 108, 110, 112, and 114 can be configured to detect a predetermined wavelength of light that is indicative of the desired reactions.

In some embodiments, the sampling device 104 includes a microcircuit arrangement, such as the microcircuit arrangement described in U.S. Pat. No. 7,595,882, which is incorporated herein by reference in its entirety. More specifically, the sampling device 104 may comprise an integrated circuit having a planar array of the sensors 106, 108, 110, 112, and 114. Circuitry formed within the sampling device 104 may be configured for at least one of signal amplification, digitization, storage, and processing. The circuitry may collect and analyze the detected fluorescent light and generate pixel signals (or detection signals) for communicating detection data to a signal processor. The circuitry may also perform additional analog and/or digital signal processing in the sampling device 104. Sampling device 104 may include conductive vias 130 that perform signal routing (e.g., transmit the pixel signals to the signal processor). The pixel signals may also be transmitted through electrical contacts of the sampling device 104.

The sampling device 104 is discussed in further details with respect to U.S. Nonprovisional patent application Ser. No. 16/874,599, titled “Systems and Devices for Characterization and Performance Analysis of Pixel-Based Sequencing,” filed May 14, 2020 (Attorney Docket No. ILLM 1011-4/IP-1750-US), which is incorporated by reference as if fully set forth herein. The sampling device 104 is not limited to the above constructions or uses as described above. In alternative embodiments, the sampling device 104 may take other forms. For example, the sampling device 104 may comprise a CCD device, such as a CCD camera, that is coupled to a flow cell or is moved to interface with a flow cell having reaction sites therein.

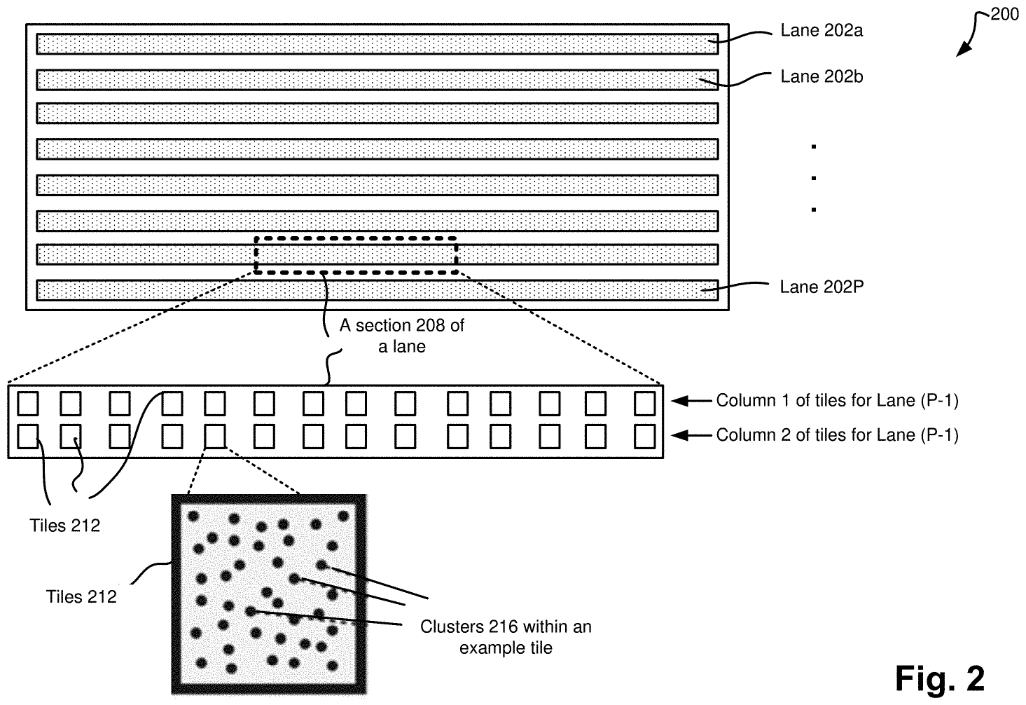

FIG. 2 depicts one implementation of a flow cell 200 that contains clusters in its tiles. The flow cell 200 corresponds to the flow cell 102 of FIG. 1, e.g., without the flow cover 136. Furthermore, the depiction of the flow cell 200 is symbolic in nature, and the flow cell 200 symbolically depicts various lanes and tiles therewithin, without illustrating various other components therewithin. FIG. 2 illustrates a top view of the flow cell 200.

In an embodiment, the flow cell 200 is divided or partitioned in a plurality of lanes, such as lanes 202a, 202b, . . . , 202P, i.e., P number of lanes. In the example of FIG. 2, the flow cell 200 is illustrated to include 8 lanes, i.e., P=8 in this example, although the number of lanes within a flow cell is implementation specific.

In an embodiment, individual lanes 202 are further partitioned into non-overlapping regions called “tiles” 212. For example, FIG. 2 illustrates a magnified view of a section 208 of an example lane. The section 208 is illustrated to comprise a plurality of tiles 212.

In an example, each lane 202 comprises one or more columns of tiles. For example, in FIG. 2, each lane 202 comprises two corresponding columns of tiles 212, as illustrated within the magnified section 208. A number of tiles within each column of tiles within each lane is implementation specific, and in one example, there can be 50 tiles, 60 tiles, 100 tiles, or another appropriate number of tiles in each column of tiles within each lane.

Each tile comprises a corresponding plurality of clusters. During the sequencing procedure, the clusters and their surrounding background on the tiles are imaged. For example, FIG. 2 illustrates example clusters 216 within an example tile.

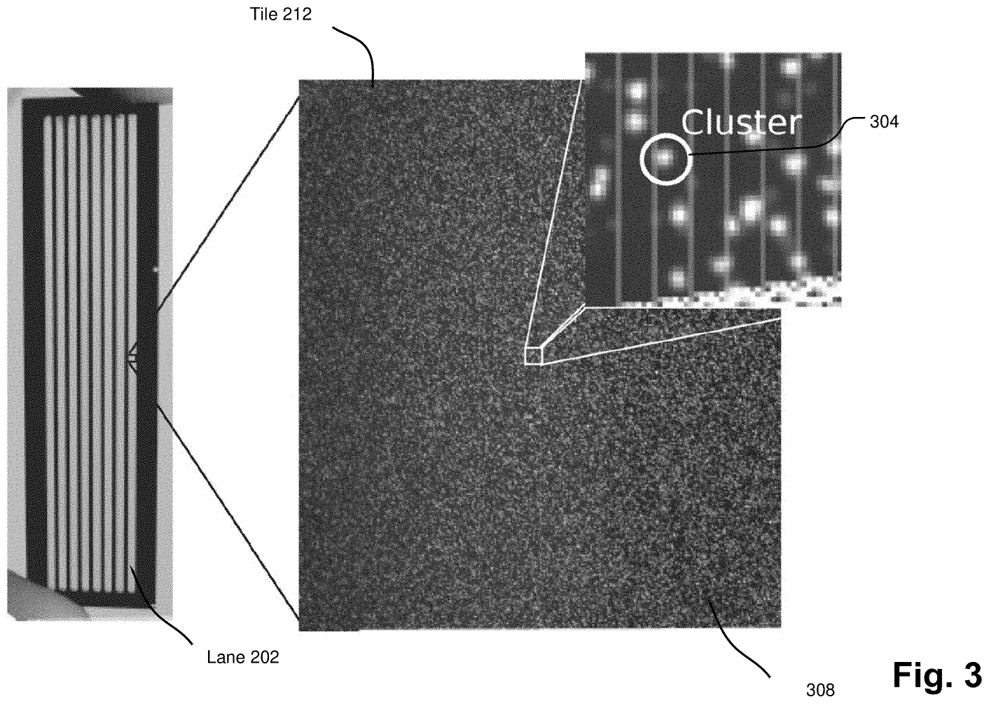

FIG. 3 illustrates an example Illumina GA-IIx™ flow cell with eight lanes, and also illustrates a zoom-in on one tile and its clusters and their surrounding background. For example, there are a hundred tiles per lane in Illumina Genome Analyzer II and sixty-eight tiles per lane in Illumina HiSeq2000. A tile 212 holds hundreds of thousands to millions of clusters. In FIG. 3, an image generated from a tile with clusters shown as bright spots is shown at 308 (e.g., 308 is a magnified image view of a tile), with an example cluster 304 labelled. A cluster 304 comprises approximately one thousand identical copies of a template molecule, though clusters vary in size and shape. The clusters are grown from the template molecule, prior to the sequencing run, by bridge amplification of the input library. The purpose of the amplification and cluster growth is to increase the intensity of the emitted signal since the imaging device cannot reliably sense a single fluorophore. However, the physical distance of the DNA fragments within a cluster 304 is small, so the imaging device perceives the cluster of fragments as a single spot 304.

The clusters and the tiles are discussed in further details with respect to U.S. Nonprovisional patent application Ser. No. 16/825,987, titled “TRAINING DATA GENERATION FOR ARTIFICIAL INTELLIGENCE-BASED SEQUENCING,” filed 20 Mar. 2020 (Attorney Docket No. ILLM 1008-16/IP-1693-US).

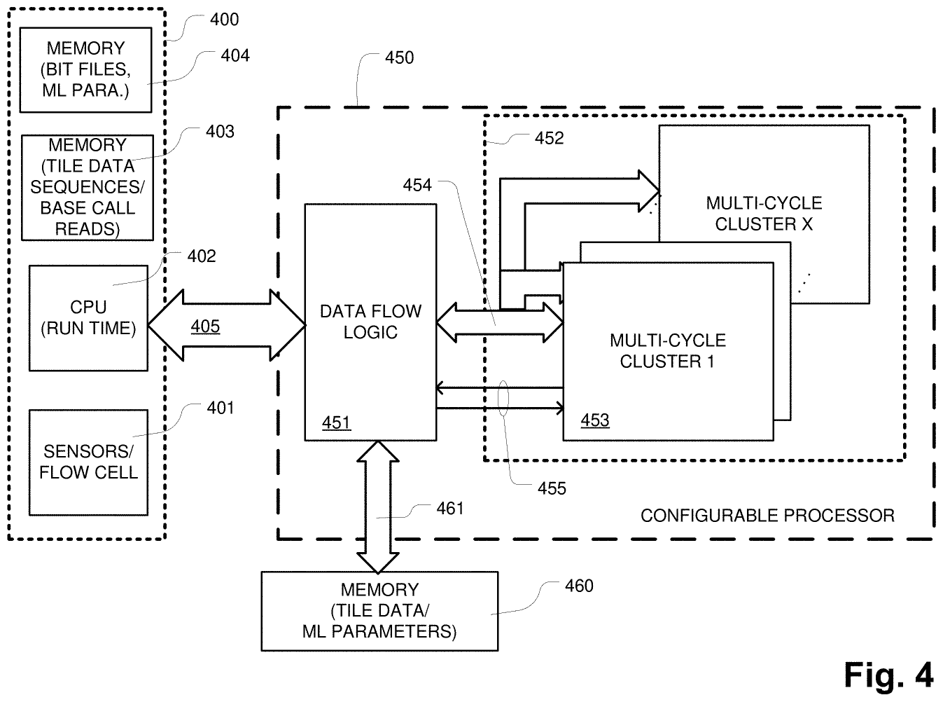

FIG. 4 is a simplified block diagram of the system for analysis of sensor data from a sequencing system, such as base call sensor outputs (e.g., see FIG. 1). In the example of FIG. 4, the system includes a sequencing machine 400 and a configurable processor 450. The configurable processor 450 can execute a neural network-based base caller in coordination with a runtime program executed by a host processor, such as a central processing unit (CPU) 402. The sequencing machine 400 comprises base call sensors and a flow cell 401 (e.g., discussed with respect to FIGS. 1-3). The flow cell can comprise one or more tiles in which clusters of genetic material are exposed to a sequence of analyte flows used to cause reactions in the clusters to identify the bases in the genetic material, as discussed with respect to FIGS. 1-3. The sensors sense the reactions for each cycle of the sequence in each tile of the flow cell to provide tile data. Examples of this technology are described in more detail below. Genetic sequencing is a data intensive operation, which translates base call sensor data into sequences of base calls for each cluster of genetic material sensed during a base call operation.

The system in this example includes the CPU 402 which executes a runtime program to coordinate the base call operations, memory 403 to store sequences of arrays of tile data, base call reads produced by the base calling operation, and other information used in the base call operations. Also, in this illustration the system includes memory 404 to store a configuration file (or files), such as FPGA bit files, and model parameters for the neural network used to configure and reconfigure the configurable processor 450 and execute the neural network. The sequencing machine 400 can include a program for configuring a configurable processor, and in some embodiments, a reconfigurable processor to execute the neural network.

The sequencing machine 400 is coupled by a bus 405 to the configurable processor 450. The bus 405 can be implemented using a high throughput technology, such as in one example bus technology compatible with the PCIe standards (Peripheral Component Interconnect Express) currently maintained and developed by the PCI-SIG (PCI Special Interest Group). Also, in this example, a memory 460 is coupled to the configurable processor 450 by a bus 461. The memory 460 can be on-board memory, disposed on a circuit board with the configurable processor 450. The memory 460 is used for high-speed access by the configurable processor 450 of working data used in the base call operation. The bus 461 can also be implemented using a high throughput technology, such as bus technology compatible with the PCIe standards.

Configurable processors, including Field Programmable Gate Arrays (FPGAs), Coarse Grained Reconfigurable Arrays (CGRAs), and other configurable and reconfigurable devices, can be configured to implement a variety of functions more efficiently or faster than might be achieved using a general-purpose processor executing a computer program. Configuration of configurable processors involves compiling a functional description to produce a configuration file, referred to sometimes as a bitstream or bit file, and distributing the configuration file to the configurable elements on the processor.

The configuration file defines the logic functions to be executed by the configurable processor, by configuring the circuit to set data flow patterns, use of distributed memory and other on-chip memory resources, lookup table contents, operations of configurable logic blocks and configurable execution units like multiply-and-accumulate units, configurable interconnects and other elements of the configurable array. A configurable processor is reconfigurable if the configuration file may be changed in the field, by changing the loaded configuration file. For example, the configuration file may be stored in volatile SRAM elements, in non-volatile read-write memory elements, and in combinations of the same, distributed among the array of configurable elements on the configurable or reconfigurable processor. A variety of commercially available configurable processors are suitable for use in a base calling operation as described herein. Examples include commercially available products such as Xilinx Alveo™ U200, Xilinx Alveo™ U250, Xilinx Alveo™ U280, Intel/Altera Stratix™ GX2800, Intel/Altera Stratix™ GX2800, and Intel Stratix™ GX10M. In some examples, a host CPU can be implemented on the same integrated circuit as the configurable processor.

Embodiments described herein implement the multi-cycle neural network using a configurable processor 450. The configuration file for a configurable processor can be implemented by specifying the logic functions to be executed using a high-level description language (HDL) or a register transfer level (RTL) language specification. The specification can be compiled using the resources designed for the selected configurable processor to generate the configuration file. The same or similar specification can be compiled for the purposes of generating a design for an application-specific integrated circuit which may not be a configurable processor.

Alternatives for the configurable processor, in all embodiments described herein, therefore include a configured processor comprising an application specific ASIC or special purpose integrated circuit or set of integrated circuits, or a system-on-a-chip SOC device, configured to execute a neural network based base call operation as described herein.

In general, configurable processors and configured processors described herein, as configured to execute runs of a neural network, are referred to herein as neural network processors.

The configurable processor 450 is configured in this example by a configuration file loaded using a program executed by the CPU 402, or by other sources, which configures the array of configurable elements on the configurable processor 450 to execute the base call function. In this example, the configuration includes data flow logic 451 which is coupled to the buses 405 and 461 and executes functions for distributing data and control parameters among the elements used in the base call operation.

Also, the configurable processor 450 is configured with base call execution logic 452 to execute a multi-cycle neural network. The logic 452 comprises a plurality of multi-cycle execution clusters (e.g., 453) which, in this example, includes multi-cycle cluster 1 through multi-cycle cluster X. The number of multi-cycle clusters can be selected according to a trade-off involving the desired throughput of the operation, and the available resources on the configurable processor.

The multi-cycle clusters are coupled to the data flow logic 451 by data flow paths 454 implemented using configurable interconnect and memory resources on the configurable processor. Also, the multi-cycle clusters are coupled to the data flow logic 451 by control paths 455 implemented using configurable interconnect and memory resources, for example, on the configurable processor, which provide control signals indicating available clusters, readiness to provide input units for execution of a run of the neural network to the available clusters, readiness to provide trained parameters for the neural network, readiness to provide output patches of base call classification data, and other control data used for execution of the neural network.

The configurable processor is configured to execute runs of a multi-cycle neural network using trained parameters to produce classification data for sensing cycles of the base flow operation. A run of the neural network is executed to produce classification data for a subject sensing cycle of the base call operation. A run of the neural network operates on a sequence including a number N of arrays of tile data from respective sensing cycles of N sensing cycles, where the N sensing cycles provide sensor data for different base call operations for one base position per operation in time sequence in the examples described herein. Optionally, some of the N sensing cycles can be out of sequence if needed according to a particular neural network model being executed. The number N can be any number greater than one. In some examples described herein, sensing cycles of the N sensing cycles represent a set of sensing cycles for at least one sensing cycle preceding the subject sensing cycle and at least one sensing cycle following the subject cycle in time sequence. Examples are described herein in which the number N is an integer equal to or greater than five.

The data flow logic 451 is configured to move tile data and at least some trained parameters of the model from the memory 460 to the configurable processor for runs of the neural network, using input units for a given run including tile data for spatially aligned patches of the N arrays. The input units can be moved by direct memory access operations in one DMA operation, or in smaller units moved during available time slots in coordination with the execution of the neural network deployed.

Tile data for a sensing cycle as described herein can comprise an array of sensor data having one or more features. For example, the sensor data can comprise two images which are analyzed to identify one of four bases at a base position in a genetic sequence of DNA, RNA, or other genetic material. The tile data can also include metadata about the images and the sensors. For example, in embodiments of the base calling operation, the tile data can comprise information about alignment of the images with the clusters such as distance from center information indicating the distance of each pixel in the array of sensor data from the center of a cluster of genetic material on the tile.

During execution of the multi-cycle neural network as described below, tile data can also include data produced during execution of the multi-cycle neural network, referred to as intermediate data, which can be reused rather than recomputed during a run of the multi-cycle neural network. For example, during execution of the multi-cycle neural network, the data flow logic can write intermediate data to the memory 460 in place of the sensor data for a given patch of an array of tile data. Embodiments like this are described in more detail below.

As illustrated, a system is described for analysis of base call sensor output, comprising memory (e.g., 460) accessible by the runtime program storing tile data including sensor data for a tile from sensing cycles of a base calling operation. Also, the system includes a neural network processor, such as configurable processor 450 having access to the memory. The neural network processor is configured to execute runs of a neural network using trained parameters to produce classification data for sensing cycles. As described herein, a run of the neural network is operating on a sequence of N arrays of tile data from respective sensing cycles of N sensing cycles, including a subject cycle, to produce the classification data for the subject cycle. The data flow logic 451 is provided to move tile data and the trained parameters from the memory to the neural network processor for runs of the neural network using input units including data for spatially aligned patches of the N arrays from respective sensing cycles of N sensing cycles.

Also, a system is described in which the neural network processor has access to the memory, and includes a plurality of execution clusters, the execution logic clusters in the plurality of execution clusters configured to execute a neural network. The data flow logic has access to the memory and to execution clusters in the plurality of execution clusters, to provide input units of tile data to available execution clusters in the plurality of execution clusters, the input units including a number N of spatially aligned patches of arrays of tile data from respective sensing cycles, including a subject sensing cycle, and to cause the execution clusters to apply the N spatially aligned patches to the neural network to produce output patches of classification data for the spatially aligned patch of the subject sensing cycle, where N is greater than 1.

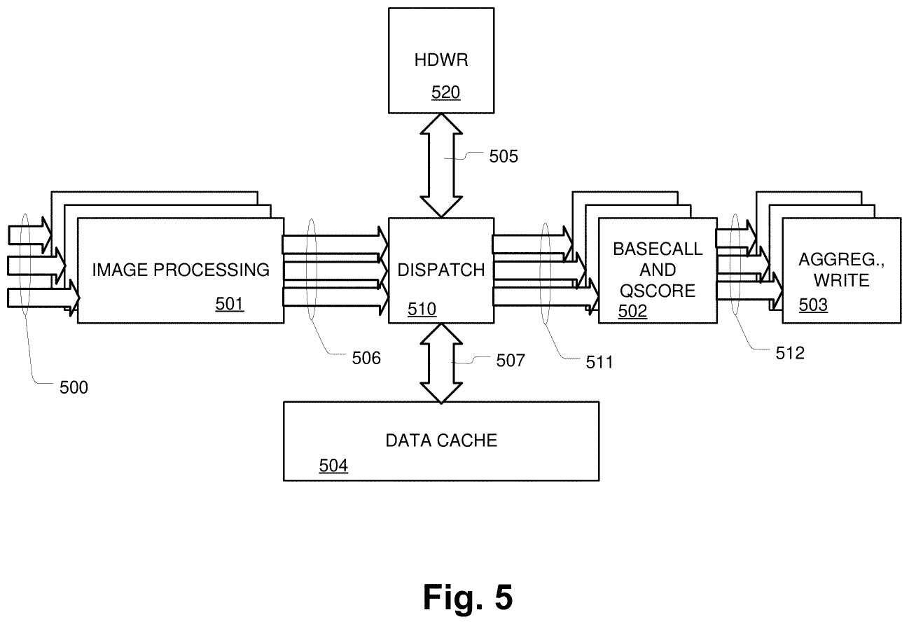

FIG. 5 is a simplified diagram showing aspects of the base calling operation, including functions of a runtime program executed by a host processor. In this diagram, the output of image sensors from a flow cell (such as those illustrated in FIGS. 1-2) are provided on lines 500 to image processing threads 501, which can perform processes on images such as resampling, alignment and arrangement in an array of sensor data for the individual tiles, and can be used by processes which calculate a tile cluster mask for each tile in the flow cell, which identifies pixels in the array of sensor data that correspond to clusters of genetic material on the corresponding tile of the flow cell. To compute a cluster mask, one example algorithm is based on a process to detect clusters which are unreliable in the early sequencing cycles using a metric derived from the softmax output, and then the data from those wells/clusters are discarded, and no output data is produced for those clusters. For example, a process can identify clusters with high reliability during the first N1 (e.g., 25) base-calls, and reject the others. Rejected clusters might be polyclonal or very weak intensity or obscured by fiducials. This procedure can be performed on the host CPU. In alternative implementations, this information would potentially be used to identify the necessary clusters of interest to be passed back to the CPU, thereby limiting the storage required for intermediate data.

The outputs of the image processing threads 501 are provided on lines 502 to a dispatch logic 510 in the CPU which routes the arrays of tile data to a data cache 504 on a high-speed bus 503, or on high-speed bus 505 to the multi-cluster neural network processor hardware 520, such as the configurable processor of FIG. 4, according to the state of the base calling operation. The hardware 520 returns classification data output by the neural network to the dispatch logic 510, which passes the information to the data cache 504, or on lines 511 to threads 502 that perform base call and quality score computations using the classification data, and can arrange the data in standard formats for base call reads. The outputs of the threads 502 that perform base calling and quality score computations are provided on lines 512 to threads 503 that aggregate the base call reads, perform other operations such as data compression, and write the resulting base call outputs to specified destinations for utilization by the customers.

In some embodiments, the host can include threads (not shown) that perform final processing of the output of the hardware 520 in support of the neural network. For example, the hardware 520 can provide outputs of classification data from a final layer of the multi-cluster neural network. The host processor can execute an output activation function, such as a softmax function, over the classification data to configure the data for use by the base call and quality score threads 502. Also, the host processor can execute input operations (not shown), such as resampling, batch normalization or other adjustments of the tile data prior to input to the hardware 520.

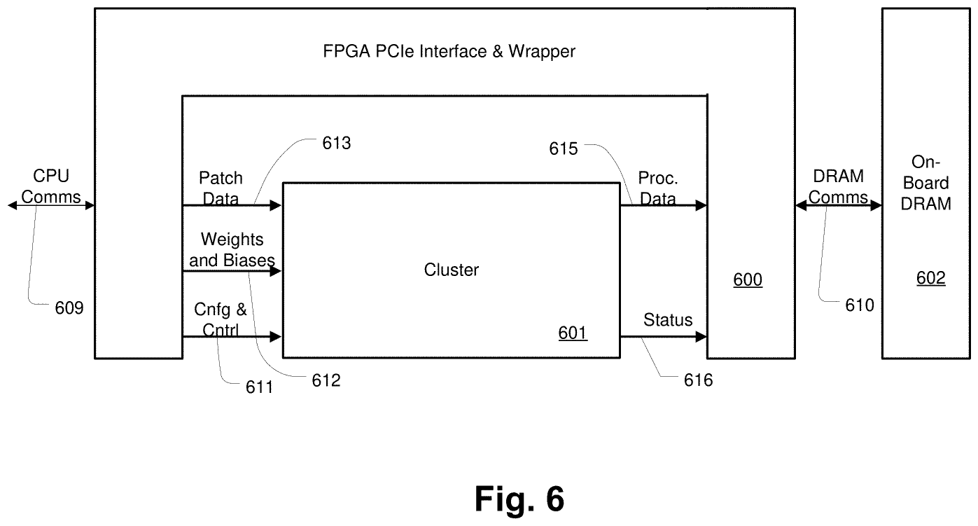

FIG. 6 is a simplified diagram of a configuration of a configurable processor such as that of FIG. 4. In FIG. 6, the configurable processor comprises an FPGA with a plurality of high speed PCIe interfaces. The FPGA is configured with a wrapper 600 which comprises the data flow logic described with reference to FIG. 1. The wrapper 600 manages the interface and coordination with a runtime program in the CPU across the CPU communication link 609 and manages communication with the on-board DRAM 602 (e.g., memory 460) via DRAM communication link 610. The data flow logic in the wrapper 600 provides patch data retrieved by traversing the arrays of tile data on the on-board DRAM 602 for the number N cycles to a cluster 601 and retrieves process data 615 from the cluster 601 for delivery back to the on-board DRAM 602. The wrapper 600 also manages transfer of data between the on-board DRAM 602 and host memory, for both the input arrays of tile data, and for the output patches of classification data. The wrapper transfers patch data on line 613 to the allocated cluster 601. The wrapper 600 provides trained parameters, such as weights and biases on line 612 to the cluster 601 retrieved from the on-board DRAM 602. The wrapper 600 provides configuration and control data on line 611 to the cluster 601 provided from, or generated in response to, the runtime program on the host via the CPU communication link 609. The cluster can also provide status signals on line 616 to the wrapper 600, which are used in cooperation with control signals from the host to manage traversal of the arrays of tile data to provide spatially aligned patch data, and to execute the multi-cycle neural network over the patch data using the resources of the cluster 601.

As mentioned above, there can be multiple clusters on a single configurable processor managed by the wrapper 600 configured for executing on corresponding ones of multiple patches of the tile data. Each cluster can be configured to provide classification data for base calls in a subject sensing cycle using the tile data of multiple sensing cycles described herein.

In examples of the system, model data, including kernel data like filter weights and biases can be sent from the host CPU to the configurable processor, so that the model can be updated as a function of cycle number. A base calling operation can comprise, for a representative example, on the order of hundreds of sensing cycles. Base calling operation can include paired end reads in some embodiments. For example, the model trained parameters may be updated once every 20 cycles (or other number of cycles), or according to update patterns implemented for particular systems and neural network models. In some embodiments including paired end reads in which a sequence for a given string in a genetic cluster on a tile includes a first part extending from a first end down (or up) the string, and a second part extending from a second end up (or down) the string, the trained parameters can be updated on the transition from the first part to the second part.

In some examples, image data for multiple cycles of sensing data for a tile can be sent from the CPU to the wrapper 600. The wrapper 600 can optionally do some pre-processing and transformation of the sensing data and write the information to the on-board DRAM 602. The input tile data for each sensing cycle can include arrays of sensor data including on the order of 4000×3000 pixels per sensing cycle per tile or more, with two features representing colors of two images of the tile, and one or two bytes per feature per pixel. For an embodiment in which the number N is three sensing cycles to be used in each run of the multi-cycle neural network, the array of tile data for each run of the multi-cycle neural network can consume on the order of hundreds of megabytes per tile. In some embodiments of the system, the tile data also includes an array of DFC data, stored once per tile, or other type of metadata about the sensor data and the tiles.

In operation, when a multi-cycle cluster is available, the wrapper allocates a patch to the cluster. The wrapper fetches a next patch of tile data in the traversal of the tile and sends it to the allocated cluster along with appropriate control and configuration information. The cluster can be configured with enough memory on the configurable processor to hold a patch of data including patches from multiple cycles in some systems, that is being worked on in place, and a patch of data that is to be worked on when the current patch of processing is finished using a ping-pong buffer technique or raster scanning technique in various embodiments.

When an allocated cluster completes its run of the neural network for the current patch and produces an output patch, it will signal the wrapper. The wrapper will read the output patch from the allocated cluster, or alternatively the allocated cluster will push the data out to the wrapper. Then the wrapper will assemble output patches for the processed tile in the DRAM 602. When the processing of the entire tile has been completed, and the output patches of data transferred to the DRAM, the wrapper sends the processed output array for the tile back to the host/CPU in a specified format. In some embodiments, the on-board DRAM 602 is managed by memory management logic in the wrapper 600. The runtime program can control the sequencing operations to complete analysis of all the arrays of tile data for all the cycles in the run in a continuous flow to provide real time analysis.

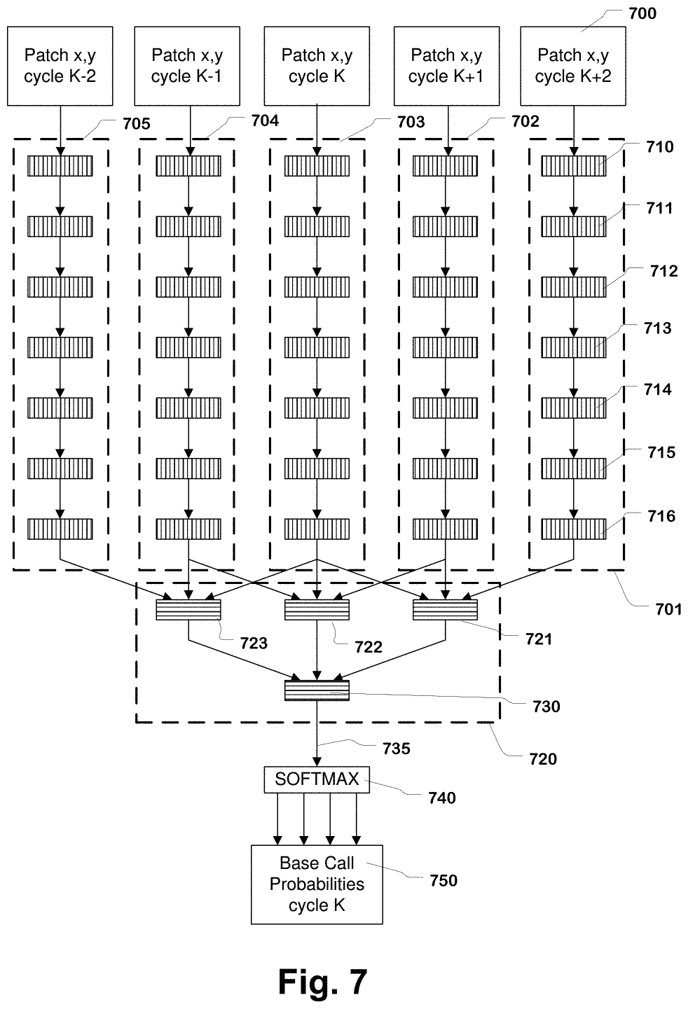

FIG. 7 is a diagram of a multi-cycle neural network model which can be executed using the system described herein. The example shown in FIG. 7 can be referred to as a five-cycle input, one-cycle output neural network. The inputs to the multi-cycle neural network model include five spatially aligned patches (e.g., 700) from the tile data arrays of five sensing cycles of a given tile. Spatially aligned patches have the same aligned row and column dimensions (x,y) as other patches in the set, so that the information relates to the same clusters of genetic material on the tile in sequence cycles. In this example, a subject patch is a patch from the array of tile data for cycle K. The set of five spatially aligned patches includes a patch from cycle K−2 preceding the subject patch by two cycles, a patch from cycle K−1 preceding the subject patch by one cycle, a patch from cycle K+1 following the patch from the subject cycle by one cycle, and a patch from cycle K+2 following the patch from the subject cycle by two cycles.

The model includes a segregated stack 701 of layers of the neural network for each of the input patches. Thus, stack 701 receives, as input, tile data for the patch from cycle K+2, and is segregated from the stacks 702, 703, 704, and 705 so they do not share input data or intermediate data. In some embodiments, all of the stacks 710-705 can have identical models, and identical trained parameters. In other embodiments, the models and trained parameters may be different in the different stacks. Stack 702 receives as input, tile data for the patch from cycle K+1. Stack 703 receives as input, tile data for the patch from cycle K. Stack 704 receives, as input, tile data for the patch from cycle K−1. Stack 705 receives as input, tile data for the patch from cycle K−2. The layers of the segregated stacks each execute a convolution operation of a kernel including a plurality of filters over the input data for the layer. As in the example above, the patch 700 may include three features. The output of the layer 710 may include many more features, such as 10 to 20 features. Likewise, the outputs of each of layers 711 to 716 can include any number of features suitable for a particular implementation. The parameters of the filters are trained parameters for the neural network, such as weights and biases. The output feature set (intermediate data) from each of the stacks 701-705 is provided as input to an inverse hierarchy 720 of temporal combinatorial layers, in which the intermediate data from the multiple cycles is combined. In the example illustrated, the inverse hierarchy 720 includes a first layer including three combinatorial layers 721, 722, 723, each receiving intermediate data from three of the segregated stacks, and a final layer including one combinatorial layer 730 receiving intermediate data from the three temporal layers 721, 722, 723.

The output of the final combinatorial layer 730 is an output patch of classification data for clusters located in the corresponding patch of the tile from cycle K. The output patches can be assembled into an output array of classification data for the tile for cycle K. In some embodiments, the output patches may have sizes and dimensions different from the input patches. In some embodiments, the output patches may include pixel-by-pixel data that can be filtered by the host to select cluster data.

The output classification data can then be applied to a softmax function 740 (or other output activation function) optionally executed by the host, or on the configurable processor, depending on the particular implementation. An output function different from softmax could be used (e g., making a base call output parameter according to largest output, then using a learned nonlinear mapping using context/network outputs to give base quality).

Finally, the output of the softmax function 740 can be provided as base call probabilities for cycle K (750) and stored in host memory to be used in subsequent processing. Other systems may use another function for output probability calculation, e.g., another nonlinear model.

The neural network can be implemented using a configurable processor with a plurality of execution clusters so as to complete evaluation of one tile cycle within the duration of the time interval, or close to the duration of the time interval, of one sensing cycle, effectively providing the output data in real time. Data flow logic can be configured to distribute input units of tile data and trained parameters to the execution clusters, and to distribute output patches for aggregation in memory.

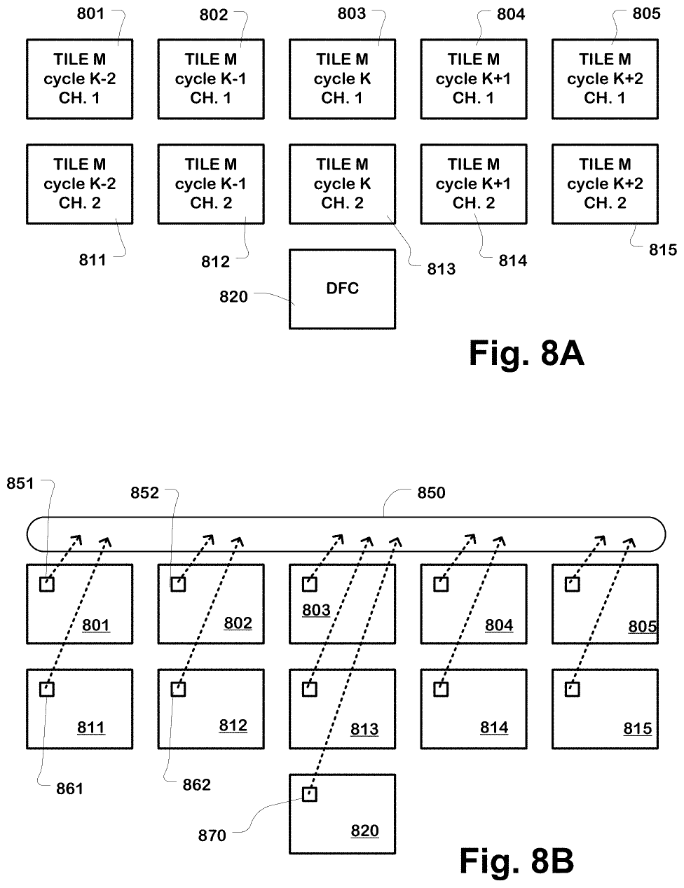

Input units of data for a five-cycle input, one-cycle output neural network like that of FIG. 7 are described with reference to FIGS. 8A and 8B for a base call operation using two-channel sensor data. For example, for a given base in a genetic sequence, the base call operation can execute two flows of analyte and two reactions that generate two channels of signals, such as images, which can be processed to identify which one of four bases is located at a current position in the genetic sequence for each cluster of genetic material. In other systems, a different number of channels of sensing data may be utilized. For example, base calling can be performed utilizing one-channel methods and systems. Incorporated materials of U.S. Patent Application Publication No. 2013/0079232 discuss base calling using various number of channels, such as one-channel, two-channels, or four-channels.

FIG. 8A shows arrays of tile data for five cycles for a given tile, tile M, used for the purposes of executing a five-cycle input, one-cycle output neural network. The five-cycle input tile data in this example can be written to the on-board DRAM, or other memory in the system which can be accessed by the data flow logic and, for cycle K−2, includes an array 801 for channel 1 and an array 811 for channel 2, for cycle K−1, an array 802 for channel 1 and an array 812 for channel 2, for cycle K, an array 803 for channel 1 and an array 813 for channel 2, for cycle K+1, an array 804 for channel 1 and an array 814 for channel 2, for cycle K+2, an array 805 for channel 1 and an array 815 for channel 2. Also an array 820 of metadata for the tile can be written once in the memory, in this case a DFC file, included for use as input to the neural network along with each cycle.

Although FIG. 8A discusses two-channel base calling operations, using two channels is merely an example, and base calling can be performed using any other appropriate number of channels. For example, incorporated materials of U.S. Patent Application Publication No. 2013/0079232 discuss base calling using various number of channels, such as one-channel, two-channels, or four-channels, or another appropriate number of channels.

The data flow logic composes input units, which can be understood with reference to FIG. 8B, of tile data that includes spatially aligned patches of the arrays of tile data for each execution cluster configured to execute a run of the neural network over an input patch. An input unit for an allocated execution cluster is composed by the data flow logic by reading spatially aligned patches (e.g., 851, 852, 861, 862, 870) from each of the arrays 801-805, 811, 815, 820 of tile data for the five input cycles, and delivering them via data paths (schematically 850) to memory on the configurable processor configured for use by the allocated execution cluster. The allocated execution cluster executes a run of the five-cycle input/one-cycle output neural network, and delivers an output patch for the subject cycle K of classification data for the same patch of the tile in the subject cycle K.

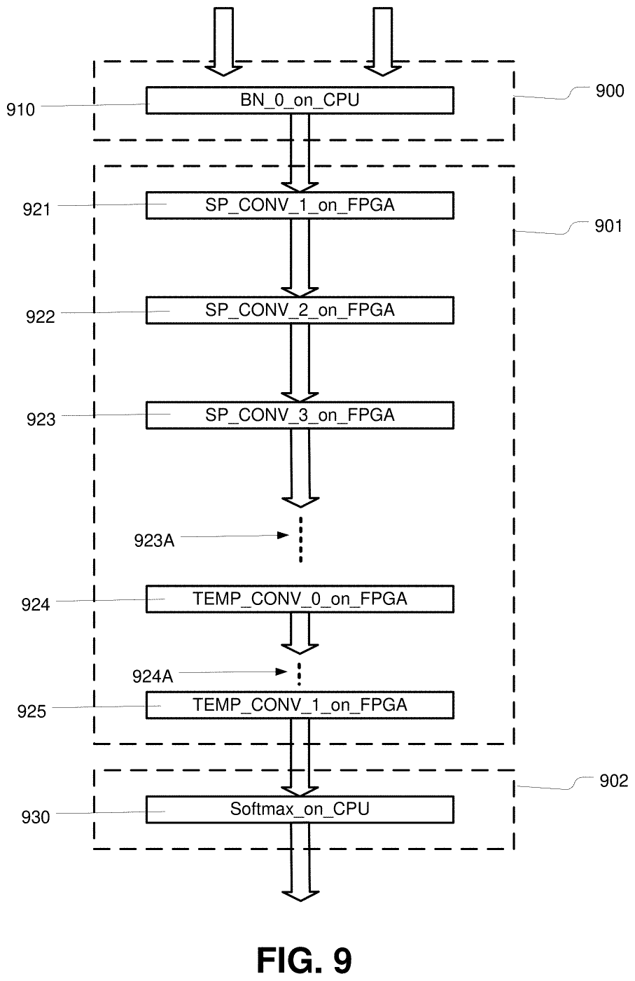

FIG. 9 is a simplified representation of a stack of a neural network usable in a system like that of FIG. 7 (e.g., 701 and 720). In this example, some functions of the neural network (e.g., 900, 902) are executed on the host, and other portions of the neural network (e.g., 901) are executed on the configurable processor.

In an example, a first function can be batch normalization (layer 910) formed on the CPU. However, in another example, batch normalization as a function may be fused into one or more layers, and no separate batch normalization layer may be present.

A number of spatial, segregated convolution layers are executed as a first set of convolution layers of the neural network, as discussed above on the configurable processor. In this example, the first set of convolution layers applies 2D convolutions spatially.

As shown in FIG. 9, a first spatial convolution 921 is executed, followed by a second spatial convolution 922, followed by a third spatial convolution 923, and so on for a number L/2 of spatially segregated neural network layers in each stack (L is described with reference to FIG. 7). As indicated at 923A, the number of spatial layers can be any practical number, which for context may range from a few to more than 20 in different embodiments.

For SP_CONV_0, kernel weights are stored for example in a (1,6,6,3,L) structure since there are 3 input channels to this layer. In this example, the “6” in this structure is due to storing coefficients in the transformed Winograd domain (the kernel size is 3×3 in the spatial domain but expands in the transform domain).

For other SP_CONV layers, kernel weights are stored for this example in a (1,6,6 L) structure since there are K(=L) inputs and outputs for each of these layers.

The outputs of the stack of spatial layers are provided to temporal layers, including convolution layers 924, 925 executed on the FPGA. Layers 924 and 925 can be convolution layers applying 1D convolutions across cycles. As indicated at 924A, the number of temporal layers can be any practical number, which for context may range from a few to more than 20 in different embodiments.

The first temporal layer, TEMP_CONVO layer 824, reduces the number of cycle channels from 5 to 3, as illustrated in FIG. 7. The second temporal layer, layer 925, reduces the number of cycle channels from 3 to 1 as illustrated in FIG. 7, and reduces the number of feature maps to four outputs for each pixel, representing confidence in each base call.

The output of the temporal layers is accumulated in output patches and delivered to the host CPU to apply for example, a softmax function 930, or other function to normalize the base call probabilities.

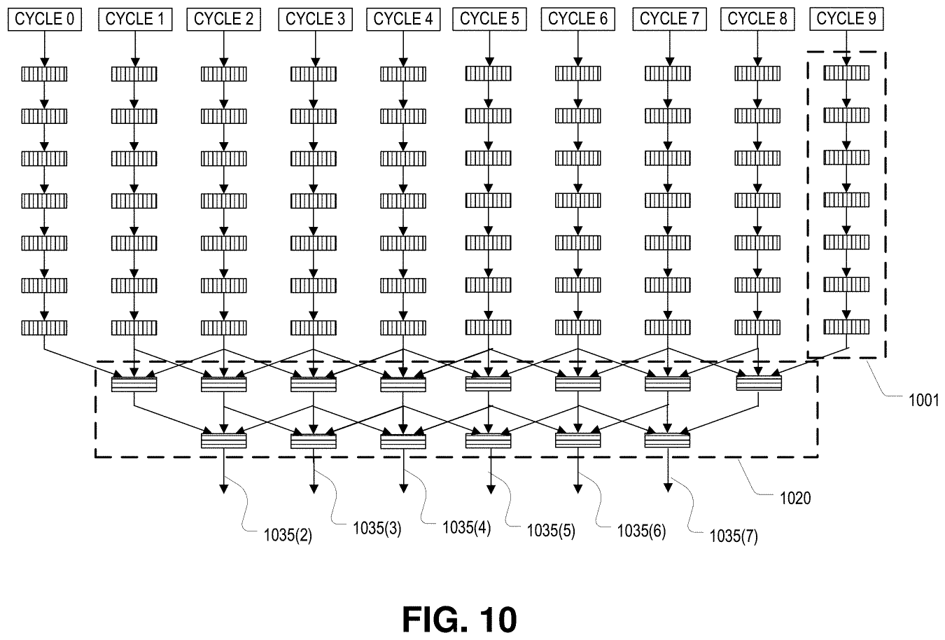

FIG. 10 illustrates an alternative implementation showing a 10-input, six-output neural network which can be executed for a base calling operation. In this example, tile data for spatially aligned input patches from cycles 0 to 9 are applied to segregated stacks of spatial layers, such as stack 1001 for cycle 9. The outputs of the segregated stacks are applied to an inverse hierarchical arrangement of temporal stacks 1020, having outputs 1035(2) through 1035(7) providing base call classification data for subject cycles 2 through 7.

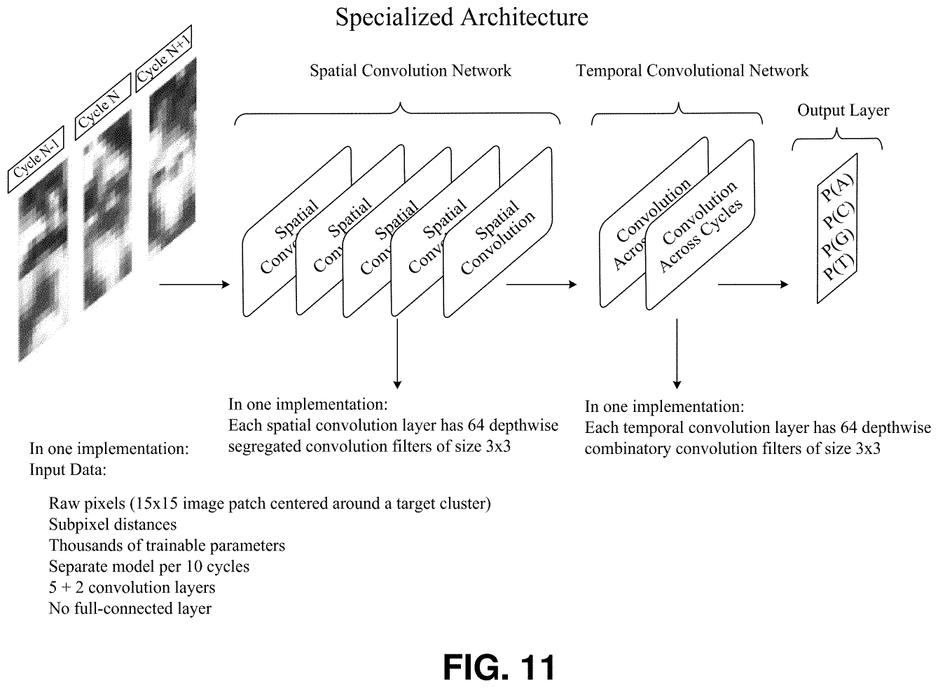

FIG. 11 illustrates one implementation of the specialized architecture of the neural network-based base caller (e.g., FIG. 7) that is used to segregate processing of data for different sequencing cycles. The motivation for using the specialized architecture is described first.

The neural network-based base caller processes data for a current sequencing cycle, one or more preceding sequencing cycles, and one or more successive sequencing cycles. Data for additional sequencing cycles provides sequence-specific context. The neural network-based base caller learns the sequence-specific context during training and base call them. Furthermore, data for pre and post sequencing cycles provides second order contribution of pre-phasing and phasing signals to the current sequencing cycle.

Images captured at different sequencing cycles and in different image channels are misaligned and have residual registration error with respect to each other. To account for this misalignment, the specialized architecture comprises spatial convolution layers that do not mix information between sequencing cycles and only mix information within a sequencing cycle.

Spatial convolution layers use so-called “segregated convolutions” that operationalize the segregation by independently processing data for each of a plurality of sequencing cycles through a “dedicated, non-shared” sequence of convolutions. The segregated convolutions convolve over data and resulting feature maps of only a given sequencing cycle, i.e., intra-cycle, without convolving over data and resulting feature maps of any other sequencing cycle.

Consider, for example, that the input data comprises (i) current data for a current (time t) sequencing cycle to be base called, (ii) previous data for a previous (time t−1) sequencing cycle, and (iii) next data for a next (time t+1) sequencing cycle. The specialized architecture then initiates three separate data processing pipelines (or convolution pipelines), namely, a current data processing pipeline, a previous data processing pipeline, and a next data processing pipeline. The current data processing pipeline receives as input the current data for the current (time t) sequencing cycle and independently processes it through a plurality of spatial convolution layers to produce a so-called “current spatially convolved representation” as the output of a final spatial convolution layer. The previous data processing pipeline receives as input the previous data for the previous (time t−1) sequencing cycle and independently processes it through the plurality of spatial convolution layers to produce a so-called “previous spatially convolved representation” as the output of the final spatial convolution layer. The next data processing pipeline receives as input the next data for the next (time t+1) sequencing cycle and independently processes it through the plurality of spatial convolution layers to produce a so-called “next spatially convolved representation” as the output of the final spatial convolution layer.

In some implementations, the current pipeline, one or more previous pipelines, and one or more next processing pipelines are executed in parallel.

In some implementations, the spatial convolution layers are part of a spatial convolutional network (or subnetwork) within the specialized architecture.

The neural network-based base caller further comprises temporal convolution layers that mix information between sequencing cycles, i.e., inter-cycles. The temporal convolution layers receive their inputs from the spatial convolutional network and operate on the spatially convolved representations produced by the final spatial convolution layer for the respective data processing pipelines.

The inter-cycle operability freedom of the temporal convolution layers emanates from the fact that the misalignment property, which exists in the image data fed as input to the spatial convolutional network, is purged out from the spatially convolved representations by the stack, or cascade, of segregated convolutions performed by the sequence of spatial convolution layers.

Temporal convolution layers use so-called “combinatory convolutions” that groupwise convolve over input channels in successive inputs on a sliding window basis. In one implementation, the successive inputs are successive outputs produced by a previous spatial convolution layer or a previous temporal convolution layer.

In some implementations, the temporal convolution layers are part of a temporal convolutional network (or subnetwork) within the specialized architecture. The temporal convolutional network receives its inputs from the spatial convolutional network. In one implementation, a first temporal convolution layer of the temporal convolutional network groupwise combines the spatially convolved representations between the sequencing cycles. In another implementation, subsequent temporal convolution layers of the temporal convolutional network combine successive outputs of previous temporal convolution layers.

The output of the final temporal convolution layer is fed to an output layer that produces an output. The output is used to base call one or more clusters at one or more sequencing cycles.

During a forward propagation, the specialized architecture processes information from a plurality of inputs in two stages. In the first stage, segregated convolutions are used to prevent mixing of information between the inputs. In the second stage, combinatory convolutions are used to mix information between the inputs. The results from the second stage are used to make a single inference for the plurality of inputs.

This is different than the batch mode technique where a convolution layer processes multiple inputs in a batch at the same time and makes a corresponding inference for each input in the batch. In contrast, the specialized architecture maps the plurality of inputs to the single inference. The single inference can comprise more than one prediction, such as a classification score for each of the four bases (A, C, T, and G).

In one implementation, the inputs have temporal ordering such that each input is generated at a different time step and has a plurality of input channels. For example, the plurality of inputs can include the following three inputs: a current input generated by a current sequencing cycle at time step (t), a previous input generated by a previous sequencing cycle at time step (t−1), and a next input generated by a next sequencing cycle at time step (t+1). In another implementation, each input is respectively derived from the current, previous, and next inputs by one or more previous convolution layers and includes k feature maps.

In one implementation, each input can include the following five input channels: a red image channel (in red), a red distance channel (in yellow), a green image channel (in green), a green distance channel (in purple), and a scaling channel (in blue). In another implementation, each input can be in blue and violet color channels (or one or more other appropriate color channels), instead of or in addition to red and green channels. In another implementation, each input can be in blue and violet color channels, instead of or in addition to red, green, purple, and/or yellow channels. In another implementation, each input can include k feature maps produced by a previous convolution layer and each feature map is treated as an input channel. In yet another example, each input can have merely one channel, two channels, or another different number of channels. Incorporated materials of U.S. Patent Application Publication No. 2013/0079232 discuss base calling using various number of channels, such as one-channel, two-channels, or four-channels.

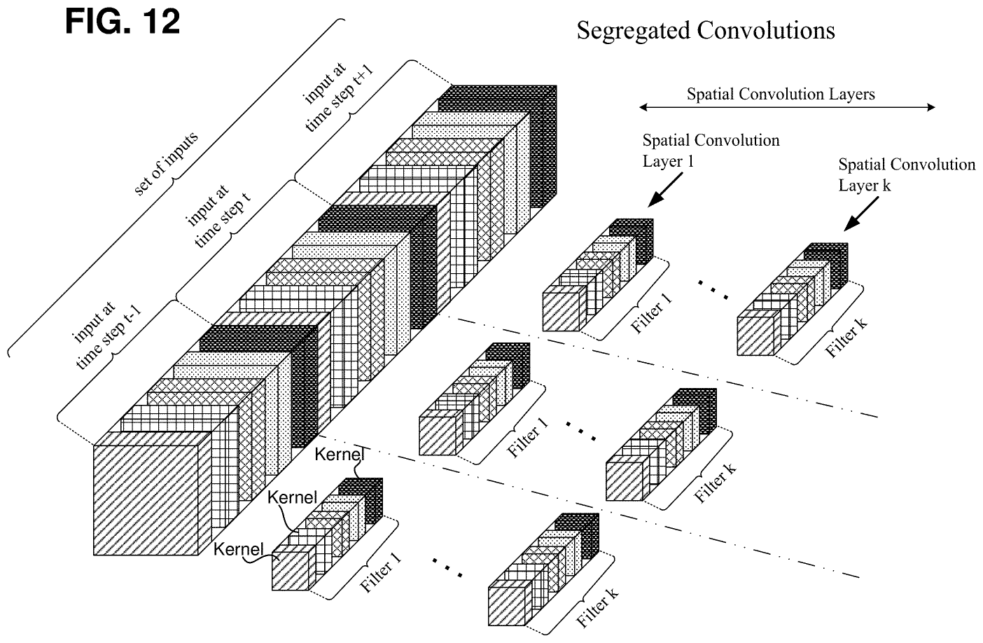

FIG. 12 depicts one implementation of segregated layers, each of which can include convolutions. Segregated convolutions process the plurality of inputs at once by applying a convolution filter to each input in parallel. With the segregated convolutions, the convolution filter combines input channels in a same input and does not combine input channels in different inputs. In one implementation, a same convolution filter is applied to each input in parallel. In another implementation, a different convolution filter is applied to each input in parallel. In some implementations, each spatial convolution layer comprises a bank of k convolution filters, each of which applies to each input in parallel.

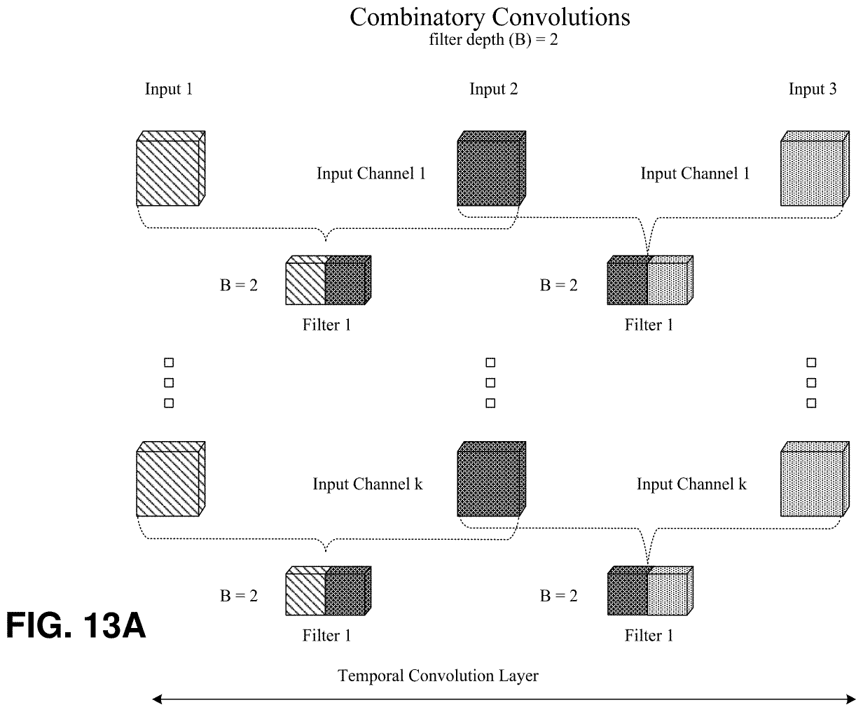

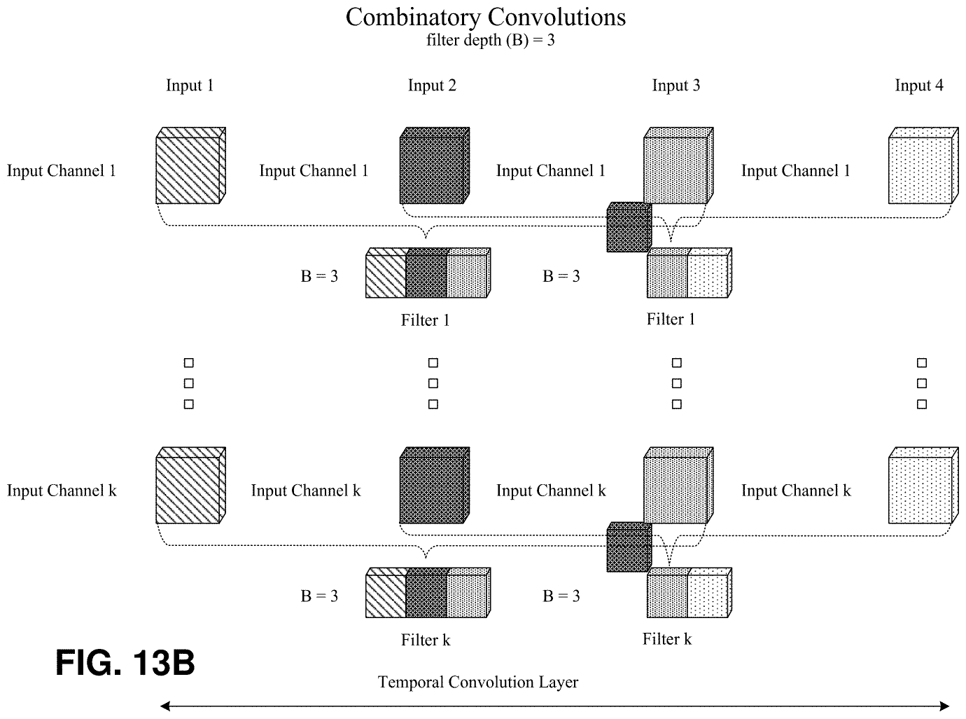

FIG. 13A depicts one implementation of combinatory layers, each of which can include convolutions. FIG. 13B depicts another implementation of the combinatory layers, each of which can include convolutions. Combinatory convolutions mix information between different inputs by grouping corresponding input channels of the different inputs and applying a convolution filter to each group. The grouping of the corresponding input channels and application of the convolution filter occurs on a sliding window basis. In this context, a window spans two or more successive input channels representing, for instance, outputs for two successive sequencing cycles. Since the window is a sliding window, most input channels are used in two or more windows.

In some implementations, the different inputs originate from an output sequence produced by a preceding spatial or temporal convolution layer. In the output sequence, the different inputs are arranged as successive outputs and therefore viewed by a next temporal convolution layer as successive inputs. Then, in the next temporal convolution layer, the combinatory convolutions apply the convolution filter to groups of corresponding input channels in the successive inputs.

In one implementation, the successive inputs have temporal ordering such that a current input is generated by a current sequencing cycle at time step (t), a previous input is generated by a previous sequencing cycle at time step (t−1), and a next input is generated by a next sequencing cycle at time step (t+1). In another implementation, each successive input is respectively derived from the current, previous, and next inputs by one or more previous convolution layers and includes k feature maps.

In one implementation, each input can include the following five input channels: a red image channel (in red), a red distance channel (in yellow), a green image channel (in green), a green distance channel (in purple), and a scaling channel (in blue). In another implementation, each input can include k feature maps produced by a previous convolution layer and each feature map is treated as an input channel.

The depth B of the convolution filter is dependent upon the number of successive inputs whose corresponding input channels are groupwise convolved by the convolution filter on a sliding window basis. In other words, the depth B is equal to the number of successive inputs in each sliding window and the group size.

In FIG. 13A, corresponding input channels from two successive inputs are combined in each sliding window, and therefore B=2. In FIG. 13B, corresponding input channels from three successive inputs are combined in each sliding window, and therefore B=3.

In one implementation, the sliding windows share a same convolution filter. In another implementation, a different convolution filter is used for each sliding window. In some implementations, each temporal convolution layer comprises a bank of k convolution filters, each of which applies to the successive inputs on a sliding window basis.

Further detail of FIGS. 4-10, and variations thereof, can be found in co-pending U.S. Nonprovisional patent application Ser. No. 17/176,147, titled “HARDWARE EXECUTION AND ACCELERATION OF ARTIFICIAL INTELLIGENCE-BASED BASE CALLER,” filed Feb. 15, 2021 (Attorney Docket No. ILLM 1020-2/IP-1866-US), which is incorporated by reference as if fully set forth herein.

### Base Calling System Generating Quality Scores

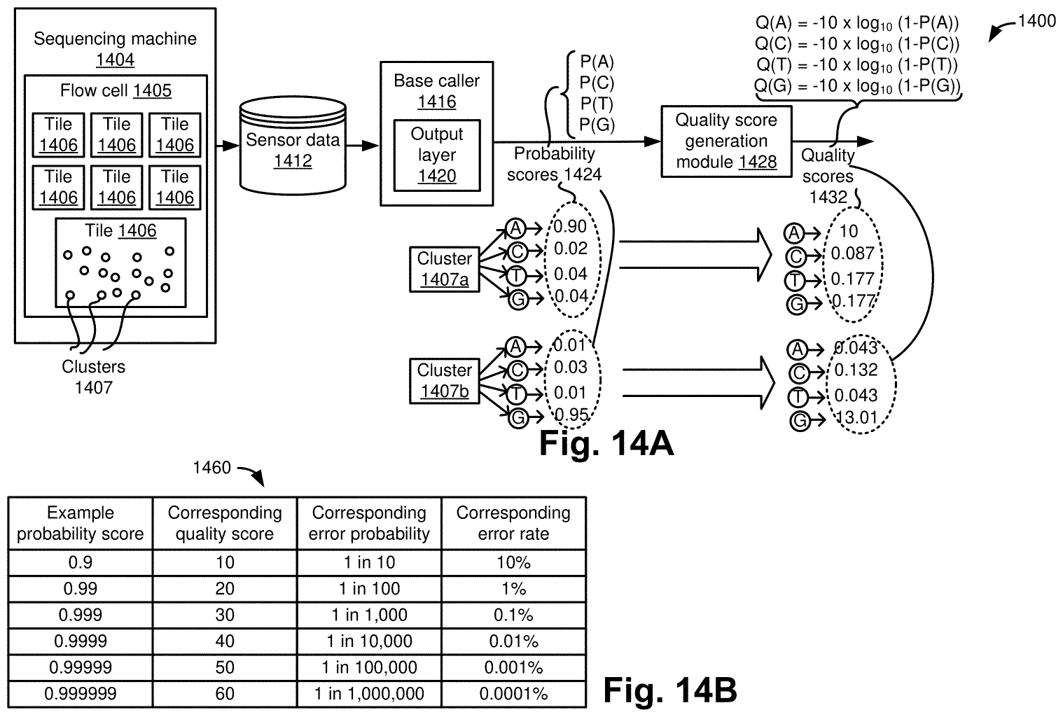

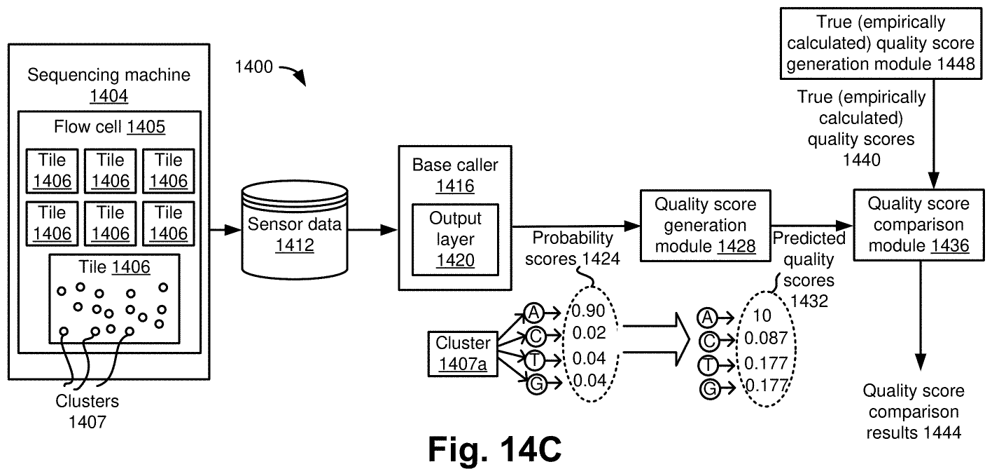

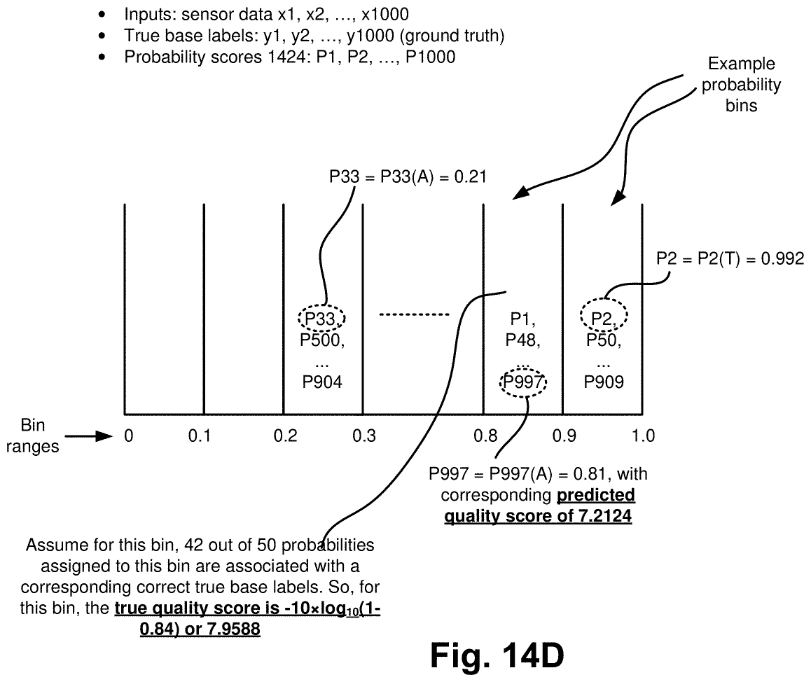

FIG. 14A illustrates a base calling system 1400 generating quality scores corresponding to A, C, T, and G for various bases to be called.

In the example of FIG. 14A, the base calling system 1400 comprises a sequencing machine 1404, such as the sequencing machine 400 of FIG. 4. In an embodiment, the sequencing machine 1404 includes a biosensor (not illustrated in FIG. 14A) comprising a flow cell 1405, similar to the flow cell 102 of the biosensor 100 of FIG. 1.

As discussed with respect to FIGS. 2, 3, and 6, the flow cell 1405 of the system 1400 comprises a plurality of tiles 1406, where each tile comprises a plurality of corresponding clusters 1407. For example, the flow cell 1405 comprises a plurality of lanes of tiles, with each tile including a corresponding plurality of clusters, as discussed with respect to FIG. 2. In FIG. 14A, the flow cell 1405 is illustrated to include some such example clusters 1407 of an example tile. During the base calling process, a base call (A, C, G, T) for every cluster at a specific sequencing cycle is predicted, accompanied by corresponding probability scores 1424 and/or quality scores 1432, as will be discussed in further detail herein.

As discussed herein previously, the sequencing machine 1404 generates sensor data 1412. For example, sensor data for individual clusters and for individual sequencing cycles are generated. Sensor data for a specific cluster and for a specific sequencing cycle is indicative of a base populating the specific cluster for the specific sequencing cycle.

The system 1400 comprises a base caller 1416. Based on the sensor data 1412, the base caller 1416 calls bases of the sequence loaded in the clusters. For example, during a base calling cycle, the base caller 1416 identifies a nucleotide base in a nucleic acid sequence in individual clusters. Base calling refers to the process of determining a base call (A, C, G, T) for every cluster at a specific cycle. As an example, base calling can be performed utilizing four-channel, two-channel or one-channel methods and systems described in the incorporated materials of U.S. Patent Application Publication No. 2013/0079232.

Sensor Data 1412 being Image Data

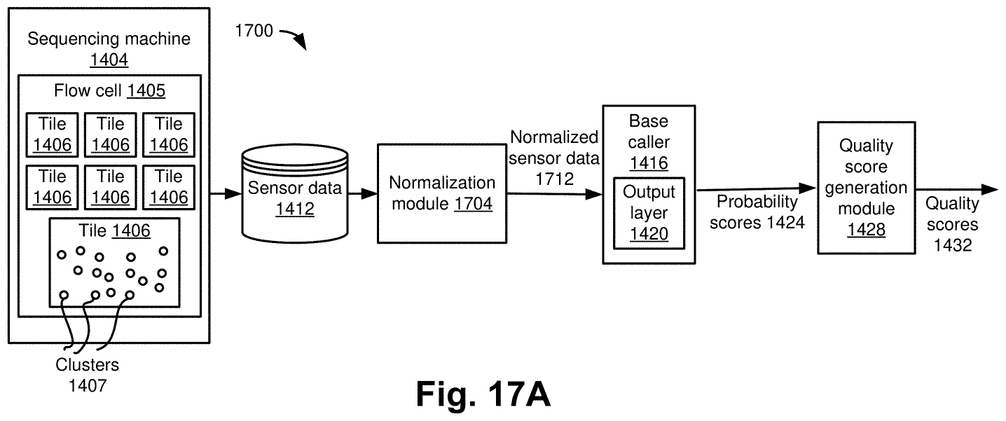

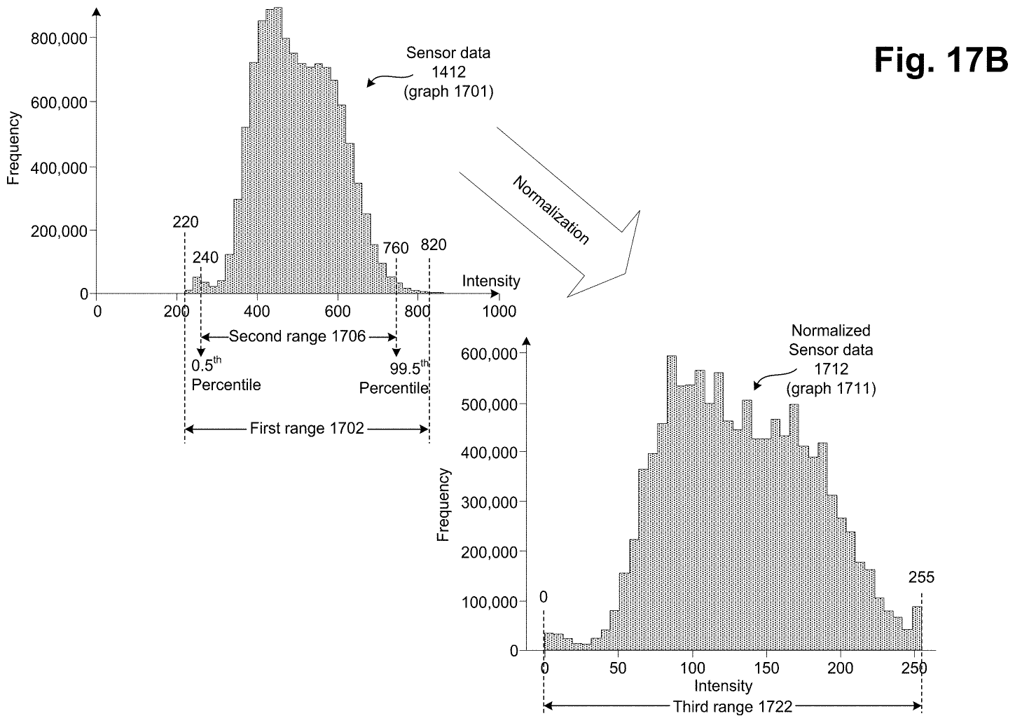

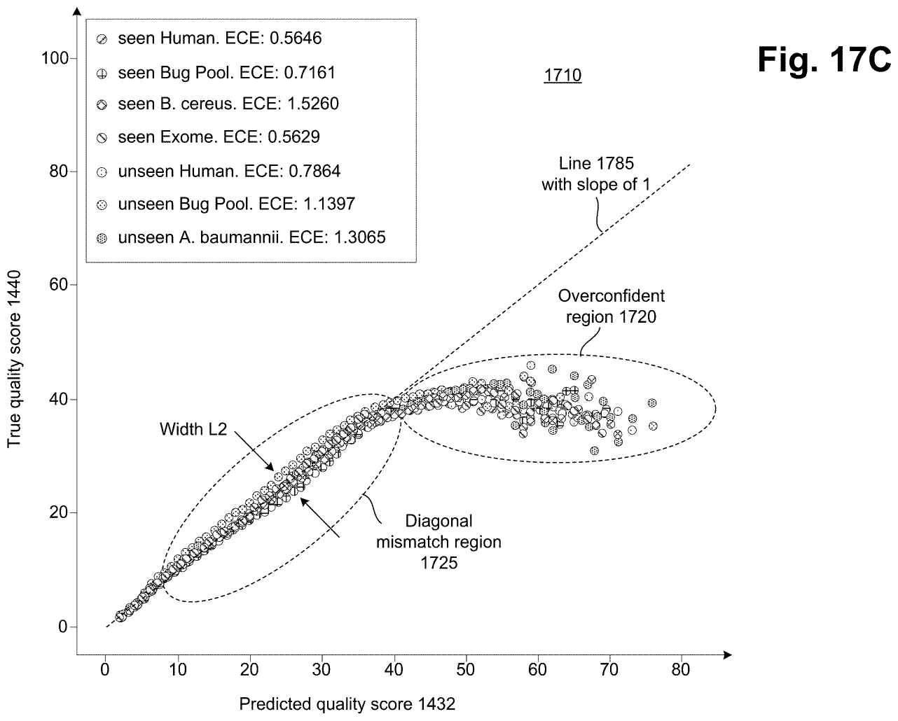

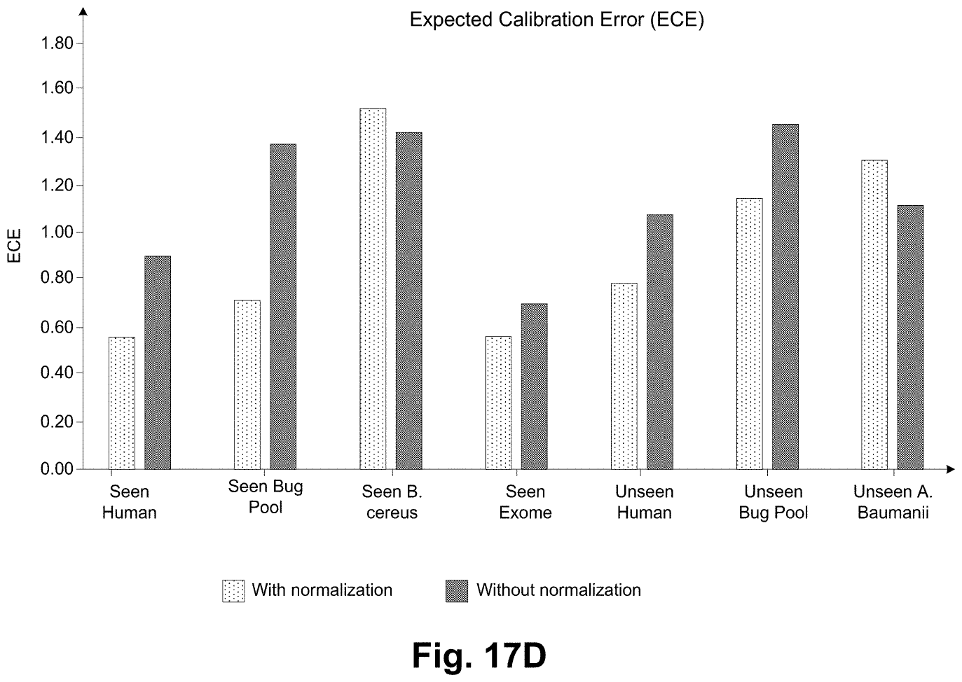

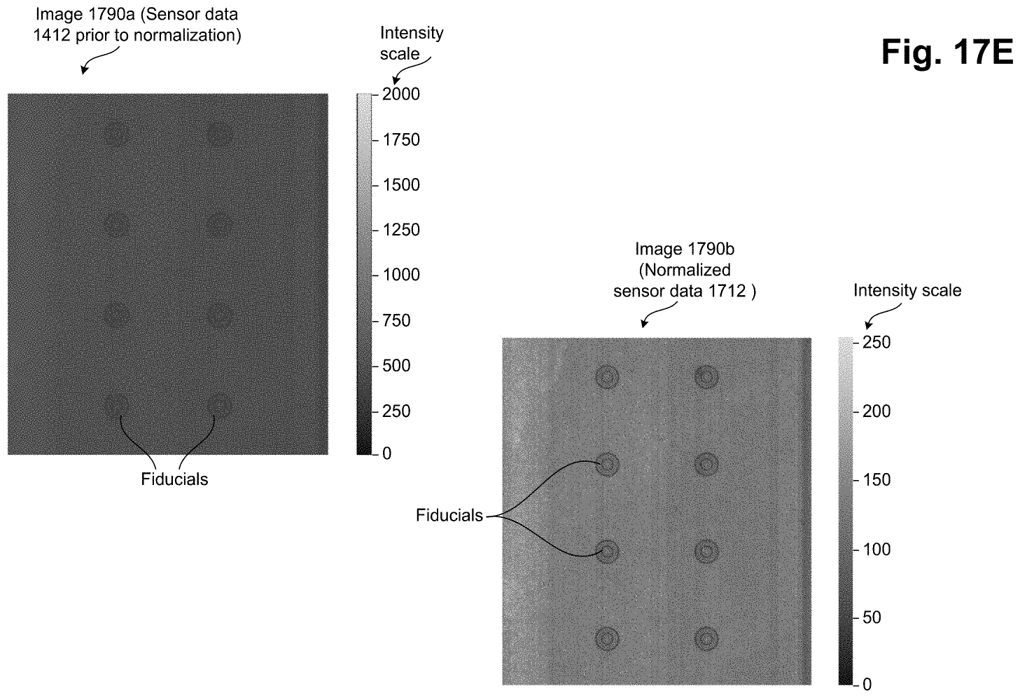

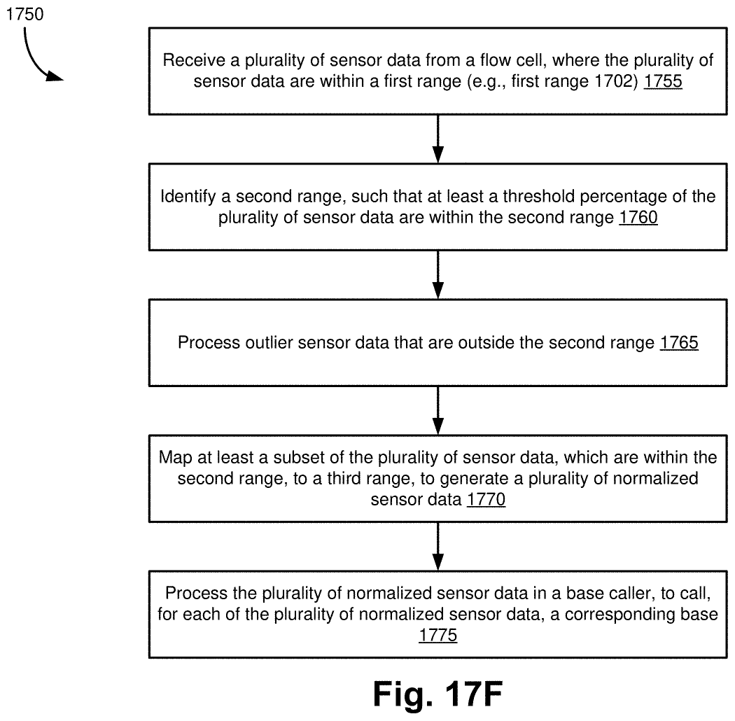

A type of sensor data 1412 generated by the sequencing machine 1404 is based on the type of sequencing machine 1404 used. For example, some of the sequencing machines discussed herein generates the sensor data 1412 in the form of images captured by sensors in the flow cell, as discussed herein previously. For example, such image data is derived from sequencing images produced by a sequencer of the sequencing machine during a sequencing run. For example, the sensor data 1412 depicts intensity emissions of a set of analytes, where intensity emissions are captured as an image (see FIG. 17E for example images comprising intensity information). As discussed, the intensity emissions are generated by analytes in the set of analytes during sequencing cycles of a sequencing run. A memory stores the images including the intensity emission of the sensor data 1412.

In one implementation, the image data comprises n×n image patches extracted from the sequencing images, where n is any number ranging between 1 and 10,000, or another appropriate range. The sequencing run produces m image(s) per sequencing cycle for corresponding m image channels, and an image patch is extracted from each of the m image(s) to prepare the image data for a particular sequencing cycle. In different implementations such as 4-, 2-, and 1-channel chemistries, m is 4 or 2. In other implementations, m is 1, 3, or greater than 4. The image data is in the optical, pixel domain in some implementations, and in the upsampled, subpixel domain in other implementations. The image data comprises data for multiple sequencing cycles (e.g., a current sequencing cycle, one or more preceding sequencing cycles, and one or more successive sequencing cycles). In one implementation, the image data comprises data for three sequencing cycles, such that data for a current (time t) sequencing cycle to be base called is accompanied with (i) data for a left flanking/context/previous/preceding/prior (time t−1) sequencing cycle and (ii) data for a right flanking/context/next/successive/subsequent (time t+1) sequencing cycle (e.g., see FIGS. 7 and 10). In other implementations, the image data comprises data for a single sequencing cycle. The image data depicts intensity emissions of one or more clusters and their surrounding background. In one implementation, when a single target cluster is to be base called, the image patches are extracted from the sequencing images in such a way that each image patch contains the center of the target cluster in its center pixel, a concept referred to herein as the “target cluster-centered patch extraction”. The image data is encoded in input data using intensity channels (also called image channels). For each of the m images obtained from the sequencer for a particular sequencing cycle, a separate image channel is used to encode its intensity data. Consider, for example, that the sequencing run uses the 2-channel chemistry which produces a red image and a green image at each sequencing cycle, then the input data comprises (i) a first red image channel with n×n pixels that depict intensity emissions of the one or more clusters and their surrounding background captured in the red image and (ii) a second green image channel with n×n pixels that depict intensity emissions of the one or more clusters and their surrounding background captured in the green image.

In an example, a biosensor comprises an array of light sensors. A light sensor is configured to sense information from a corresponding pixel area (e.g., a reaction site/well/nanowell) on the detection surface of the biosensor. An analyte disposed in a pixel area is said to be associated with the pixel area, i.e., the associated analyte. At a sequencing cycle, the light sensor corresponding to the pixel area is configured to detect/capture/sense emissions/photons from the associated analyte and, in response, generate a pixel signal for each imaged channel. In one implementation, each imaged channel corresponds to one of a plurality of filter wavelength bands. In another implementation, each imaged channel corresponds to one of a plurality of imaging events at a sequencing cycle. In yet another implementation, each imaged channel corresponds to a combination of illumination with a specific laser and imaging through a specific optical filter. Pixel signals from the light sensors are communicated to a signal processor coupled to the biosensor (e.g., via a communication port). For each sequencing cycle and each imaged channel, the signal processor produces an image whose pixels respectively depict/contain/denote/represent/characterize pixel signals obtained from the corresponding light sensors. This way, a pixel in the image corresponds to: (i) a light sensor of the biosensor that generated the pixel signal depicted by the pixel, (ii) an associated analyte whose emissions were detected by the corresponding light sensor and converted into the pixel signal, and (iii) a pixel area on the detection surface of the biosensor that holds the associated analyte. Consider, for example, that a sequencing run uses two different imaged channels: a red channel and a green channel. Then, at each sequencing cycle, the signal processor produces a red image and a green image. This way, for a series of k sequencing cycles of the sequencing run, a sequence with k pairs of red and green images is produced as output. Pixels in the red and green images (i.e., different imaged channels) have one-to-one correspondence within a sequencing cycle. This means that corresponding pixels in a pair of the red and green images depict intensity data for the same associated analyte, albeit in different imaged channels. Similarly, pixels across the pairs of red and green images have one-to-one correspondence between the sequencing cycles. This means that corresponding pixels in different pairs of the red and green images depict intensity data for the same associated analyte, albeit for different acquisition events/timesteps (sequencing cycles) of the sequencing run. Corresponding pixels in the red and green images (i.e., different imaged channels) can be considered a pixel of a “per-cycle image” that expresses intensity data in a first red channel and a second green channel. A per-cycle image whose pixels depict pixel signals for a subset of the pixel areas, i.e., a region (tile) of the detection surface of the biosensor, is called a “per-cycle tile image.” A patch extracted from a per-cycle tile image is called a “per-cycle image patch.” In one implementation, the patch extraction is performed by an input preparer. The image data comprises a sequence of per-cycle image patches generated for a series of k sequencing cycles of a sequencing run. The pixels in the per-cycle image patches contain intensity data for associated analytes and the intensity data is obtained for one or more imaged channels (e.g., a red channel and a green channel) by corresponding light sensors configured to detect emissions from the associated analytes. In one implementation, when a single target cluster is to be base called, the per-cycle image patches are centered at a center pixel that contains intensity data for a target associated analyte and non-center pixels in the per-cycle image patches contain intensity data for associated analytes adjacent to the target associated analyte. In one implementation, the image data is prepared by an input preparer.

Further detail of examples of the sensor data 1412 being image data can be found in U.S. Nonprovisional patent application Ser. No. 16/826,134, titled “ARTIFICIAL INTELLIGENCE-BASED QUALITY SCORING,” filed 20 Mar. 2020 (Attorney Docket No. ILLM 1008-19/IP-1747-US), which is incorporated by reference herein.

Sensor Data 1412 being Non-Image Data

In yet another example, the sensor data 1412 can be indicative of a chemical property (such as pH level) that, in turn, is indicative of a base to be predicted. For example, such pH changes may be induced by the release of hydrogen ions during molecule extension. The pH changes are detected and converted to a voltage change that is proportional to the number of bases incorporated (e.g., in the case of Ion Torrent).

In another example, the sensor data 1412 can be in the form of electrical signals (e g., current or voltage) generated by the flow cell 1405.

In yet another example, the sensor data 1412 is constructed from nanopore sensing that uses biosensors to measure the disruption in current as an analyte passes through a nanopore or near its aperture while determining the identity of the base. For example, the Oxford Nanopore Technologies (ONT) sequencing is based on the following concept: pass a single strand of DNA (or RNA) through a membrane via a nanopore and apply a voltage difference across the membrane. The nucleotides present in the pore will affect the pore's electrical resistance, so current measurements over time can indicate the sequence of DNA bases passing through the pore. This electrical current signal (the ‘squiggle’ due to its appearance when plotted) is the raw data gathered by an ONT sequencer. These measurements are stored as 16-bit integer data acquisition (DAC) values, taken at 4 kHz frequency (for example). With a DNA strand velocity of ˜450 base pairs per second, this gives approximately nine raw observations per base on average. This signal is then processed to identify breaks in the open pore signal corresponding to individual reads. These stretches of raw signal are base called through the process of converting DAC values into a sequence of DNA bases. In some implementations, the sensor data 1412 comprises normalized or scaled DAC values.

Base Caller 1416

The base caller 1416 can be any appropriate type of base caller. In an example, the base caller 1416 can be a neural network based base caller, which is also referred to herein as “Deep Learning” based base caller, discussed with respect to FIGS. 7-13B. In another example, the base caller is a “RTA” based base caller, which comprises a non-neural network model that is at least in part linear. Examples of a Deep Learning based base caller and an RTA base caller are discussed in U.S. Non-Provisional patent application Ser. No. 16/826,126, entitled “Artificial Intelligence-Based Base Calling,” filed 20 Mar. 2020 (Attorney Docket No. ILLM 1008-18/IP-1744-US), which is incorporated by reference for all purposes as if fully set forth herein. The principles of this disclosure are not limited to a type of base caller used to generate base calls. For example, the base caller 1416 may be of some other appropriate type, which can process any appropriate type of sensor data, such as image and/or non-image type of sensor data previously discussed herein.

In an example, the base caller 1416 is local to the sequencing machine 1404. Thus, the base caller 1416 and the sequencing machine 1404 are proximally located (e.g., within a same housing, or within two proximally located housing), and the base caller 1416 receives the sensor data 1412 directly from the sequencing machine 1404.

In another example, the base caller 1416 is located remotely relative to the sequencing machine 1404, which is an example of the so-called cloud-based base caller. Thus, the base caller 1416 receives the sensor data 1412 from the sequencing machine 1404 via a computer network, such as the Internet.

Probability Scores

In an example, irrespective of the location and/or the type of base caller used, the base caller 1416 comprises an output layer 1420 to generate probability scores of the bases to be called. For example, the output layer 1420 produces likelihoods (classification scores) of a base incorporated in the single target cluster at the current sequencing cycle being one of A, C, T, and G, and classifies the base as one of A, C, T, or G based on the likelihoods (e.g., the base with the maximum likelihood is selected). In such implementations, the likelihoods are exponentially normalized scores produced by a softmax classification layer and sum to unity. Thus, the output layer 1420, which for example may include a softmax layer, predicts a called base and corresponding probabilities P(A), P(C), P(T), P(G).

For example, for a specific base to be called corresponding to a specific cluster, corresponding probability scores 1424 are generated. Example probability scores for two example clusters 1407a and 1407b are illustrated in FIG. 14A. Merely as an example, for the cluster 1407a, a probability of the base to be called for a specific sequencing cycle being an A is P(A)=0.9; a probability of the base to be called for the specific sequencing cycle being a C is P(C)=0.02; a probability of the base to be called for the specific sequencing cycle being a T is P(T)=0.04; and a probability of the base to be called for the specific sequencing cycle being a G is P(G)=0.04.

Merely as an example, for the other cluster 1407b, a probability of the base to be called for a specific sequencing cycle being an A is P(A)=0.01; a probability of the base to be called for the specific sequencing cycle being a C is P(C)=0.03; a probability of the base to be called for the specific sequencing cycle being a T is P(T)=0.01; and a probability of the base to be called for the specific sequencing cycle being a G is P(G)=0.95.

Note that for a given cluster and for a given sequencing cycle, the sum of the probability scores P(A)+P(C)+P(T)+P(G) is 1, i.e., the probability scores are normalized (e.g., using a softmax function in, or subsequent to, output layer 1420).

In an example, the probability scores 1424 are also referred to herein as likelihood scores, softmax scores, confidence scores, and/or the like. The probability scores 1424 are generated for each cluster and for each sequencing cycle of the sequencing run.

In an embodiment, in addition to the probability scores 1424, the base caller 1416 may also call a base. Merely as an example, for the cluster 1407a, the base caller 1416 may call the base to be an A, based on the probability score P(A) being higher than a threshold value and/or based on the probability score P(A) being higher than each of P(C), P(T), or P(G). Similarly, for the cluster 1407b, the base caller 1416 may call the base to be a G, based on the probability score P(G) being higher than a threshold value and/or based on the probability score P(G) being higher than each of P(A), P(C), or P(T).

Quality Scores 1432

In an embodiment, the base calling system 1400 further comprises a quality score generation module 1428 configured to transform the probability scores 1424 to corresponding quality scores 1432. For example, a quality score Q is related a corresponding probability score P as follows:

Q=−10×log₁₀(1−P). Equation 1

Thus, for a given cluster and a given sequencing cycle and for bases A, C, T, and G, the corresponding quality scores are given as:

Q(A)=−10×log 10(1−P(A)),

Q(C)=−10×log 10(1−P(C)),

Q(T)=−10×log 10(1−P(T)),

Q(G)=−10×log₁₀(1−P(G)). Equations 2

Note that P(A), P(C), P(T), P(G) are respectively probabilities of the base being called is an A, a C, a T, or a G. Assume E(A) is an error probability associated with the base being called an A, E(C) is an error probability associated with the base being called a C, E(T) is an error probability associated with the base being called a T, and E(G) is an error probability associated with the base being called a G. Thus, E(A)=1−P(A); E(C)=1−P(C); and so on. In such an example, the quality scores can also be rewritten as:

Q(A)=−10×log₁₀(1−E(A)),

Q(C)=−10×log₁₀(1−E(C)),

Q(T)=−10×log₁₀(1−E(T)),

Q(G)=−10×log₁₀(1−E(G)). Equations 3

Referring to equations 2 and 3, the quality scores are defined as a property which is logarithmically related to the base calling probability scores P or base calling error probability scores E. Thus, a quality score Q(A) is a likelihood, in logarithmic scale, of a likelihood of a base to be called being an A; a quality score Q(C) is a likelihood, in logarithmic scale, of a likelihood of a base to be called being a C; and so on.

Often times, the quality scores Q are also referred to as “Phred” scores, and are a measure of the quality of the identification of the nucleobases generated by automated DNA sequencing machines, such as by the sequencing machine 1404.

FIG. 14A illustrates example quality scores 1422 corresponding to the probability scores 1424 for the example clusters 1407a and 1407b. For example, the cluster 1407a has a probability score P(A)=0.9 and a corresponding quality score Q(A)=10 (calculated using equations 2), has a probability score P(C)=0.02 and a corresponding quality score Q(C)=0.087, and so on. In an example, generally, quality scores are calculated for relatively higher probability scores, such as for probability scores higher than a threshold values (such as higher than 0.9), e.g., as illustrated in FIG. 14B.

FIG. 14B illustrates a table 1460 indicating the relationship between the probability scores 1424, the quality scores 1432, corresponding error probabilities, and corresponding error rates. The table 14B is derived from equations 1, 2, and 3. The table 1460 is self-explanatory.

The probabilistic interpretation of quality scores allows fair integration of different sequencing reads in downstream analysis, such as variant calling and sequence assembly. As discussed, a quality score is a measure of the probability of a sequencing error in a base call. A relatively high value of the quality score implies that a base call is more reliable and less likely to be incorrect, and vice versa. For example, as seen in the table 1460, if the quality score of a base is 30, the probability that this base is called incorrectly is 0.001. This also indicates that the base call accuracy is 99.9%.

Note that the disclosure discusses various modules, such as the quality score generation module 1428. In an example and unless otherwise mentioned, each of these modules are executed by a processor (e.g., the CPU 402 and/or the configurable processor 450, see FIG. 4). Thus, for example, computer readable instructions executable by such processor(s) cause implementation of these modules.

### Predicted Quality Scores 1432 Versus True Quality Scores 1440

FIG. 14C illustrates a comparison operation between predicted quality scores 1432 predicted by the base calling system 1400 of FIG. 14A and true (e.g., empirically calculated) quality scores 1440. For example, a true quality score generation module 1448 generates true (e.g., empirically calculated) quality scores 1440. A quality score comparison module 1436 receives the predicted quality scores 1432 that are predicted by the base calling system 1400. Note that the quality scores 1432 of FIG. 14A are referred to as predicted quality scores 1432 in FIG. 14C, to better distinguish these quality scores from the true quality scores 1440. The quality score comparison module 1436 also receives the true quality scores 1440, and compares the true quality scores 1440 with the predicted quality scores 1432, to generate quality score comparison results 1444.

### True (e.g., Empirically Determined) Quality Scores 1440

FIG. 14D illustrates determination of true (e.g., empirically determined) quality scores 1440 of FIG. 14C. For example, the true (e.g., empirically calculated) quality score generation module 1448 determines the true quality scores, e.g., by empirically calculating quality scores that are likely to be representative of a true likelihood associated with the quality scores.

In the example of FIG. 14D, assume that the base caller 1416 of FIG. 14A receives 1,000 inputs x1, x2, . . . , x1000, which are sensor data 1412. Note that the 1,000 number of samples is a non-limiting example. Also assume that the base caller 1416 generates 1,000 probability scores 1424, such as probability scores P1, P2, . . . , P1000. Each of these probability scores is associated with a corresponding base being called a corresponding one of A, C, T, or G. Merely as examples, assume that P2 is a probability P2(T) of a base being called a T, and has a value of 0.992; and assume that P33 is a probability P33(A) of a base being called an A, and has a value of 0.21, as illustrated in FIG. 14D. In an example, assume, for a base number 2, the associated probabilities are P2(A), P2(C), P2(T), and P2(G). Also assume that P2(T) is highest among P2(A), P2(C), P2(T), and P2(G). Accordingly, in the example of FIG. 14B, P2 is assumed to simply be P2(T) (and not P2(A), P2(C), or P2(G)). That is, P2 is the highest among the associated four probability scores for the base number 2. Similarly, P33 is the highest among the associated four probability scores for the base number 33, and so on.

Also assume, for inputs x1, x2, . . . , x1000, true or ground truth base labels y1, y2, . . . , y1000, respectively, are received by the true quality score generation module 1448 (i.e., true base label y1 is for input x1, true base label y2 is for input x2, and so on). A true base label is an actual ground truth base label for the base to be called. For example, assume, for input x1 generated at a specific cluster for a specific sequencing cycles, base calling probabilities P(A), P(C), P(T), and P(G) are predicted. The true base label y1 is the actual base (which can be one of A, C, T, or G) in that cluster and for that sequencing cycle. In an example, the true base labels y1, . . . , y1000 are known a-priori, e.g., by sequencing a known base sequence.

In FIG. 14D, each predicted probability score 1424 is assigned to a corresponding one of several pre-specified bins. Merely as an example, the predicted probability scores 1424 are assigned to corresponding ones of the following pre-specified bins: [0,0.1), [0.1,0.2), . . . , [0.9,1.0], as illustrated in FIG. 14D.

For example, as P33 is 0.21, the predicted probability score P33 is assigned to the bin [0.2,0.3); and as P2 is 0.992, the predicted probability score P2 is assigned to the bin [0.9,1.0]. Merely as examples, predicted probability scores P33, P500, . . . , P904 are assigned to the bin [0.2,0.3), predicted probability scores P1, P48, . . . , P997 are assigned to the bin [0.8,0.9), and predicted probability scores P2, P50, . . . , P909 are assigned to the bin [0.9,1.0].

After assigning the predicted probability scores 1424 to corresponding bins, the true quality score generation module 1448 calculates an accuracy or “true empirical likelihood” of individual bins. Assume that P2=0.992 is a prediction of T. Then the true quality score generation module 1448 checks to see if the corresponding true base label y2 is a T. If y2 is indeed a T, then the prediction P2 is correct.

This validation (or verification) process is repeated for each prediction and for each bin, e.g., to calculate a true probability of each bin. For example, assume that there are 50 probabilities P1, P48, . . . , P907 in the bin [0.8,0.9) and it is determined that 42 of those probabilities match with their corresponding true base labels y1, y48, . . . , y907, respectively. Then a “true” or empirically determined probability for that bin is 42/50 or 0.84. The true quality score 1440 for entries in that bin is then determined using equation 1. Specifically, the true quality score 1440 for entries in that bin is −10×log₁₀(1−0.84) or 7.9588. Thus, probabilities P1, P48, . . . , P907 in the bin [0.8,0.9) are assigned the true quality score of 7.9588.

In contrast, assume merely as an example, that predicted probability P997 assigned to the bin [0.8,0.9) is 0.81, which corresponds to a quality score of −10×log₁₀(1−0.81) or 7.2124.

Thus, for P997, the predicted quality score 1432 is 7.2124, whereas a true quality score 1440 is 7.9588. Thus, there is a mismatch between the predicted quality score 1432 and the true quality score 1440 for P997.

In an example, the quality score comparison module 1436 outputs quality score comparison results 1444, which compare the true quality scores 1440 with the predicted quality scores 1432, as will be discussed herein later in turn.

Note that the binning illustrated in FIG. 14D is merely an oversimplified example. For example, in FIG. 14D, the predicted probabilities are assigned among merely 10 bins. However, in another example, there can be higher number of bins to which the predicted probabilities are assigned. For example, the single bin [0.9,1.0] may be subdivided in multiple bins, such as [0.9,0.91), [0.91,0.92), . . . , [0.99,1.0].

In an example, instead of binning the predicted probabilities (as illustrated in FIG. 14D), the predicted quality scores 1432 may be binned instead. For example, the predicted quality scores 1432 are assigned in corresponding bins. Also, true quality scores for individual bins are calculated in the above discussed manner. Then the quality score comparison module 1436 can directly compare the true quality scores 1440 with the predicted quality scores 1432.

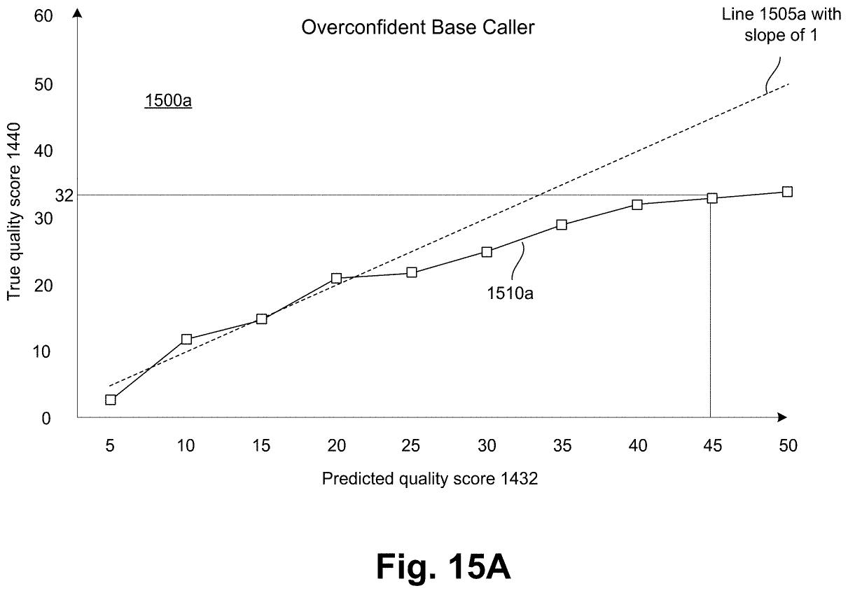

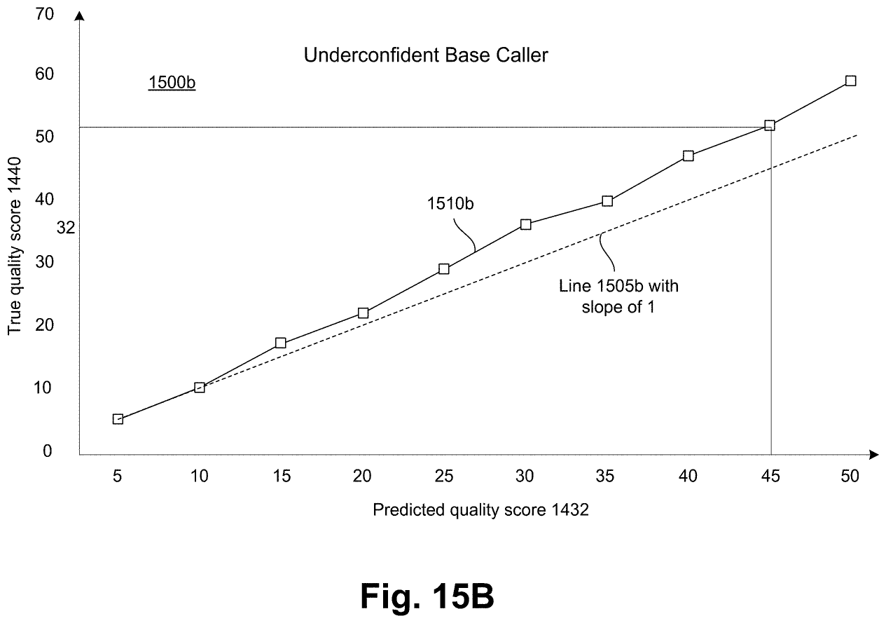

FIG. 15A illustrates a graph 1500a depicting a comparison between predicted quality scores 1432 and true quality scores 1440, and FIG. 15B illustrates another graph 1500b depicting another comparison between predicted quality scores 1432 and true quality scores 1440.

Graph 1500a has a dashed line 1505a having a slope of 1. Thus, any point on the line 1505a has equal values of predicted quality score 1432 and true quality score 1440. Similarly, graph 1500b has a dashed line 1505b having a slope of 1. Thus, any point on the line 1505b has equal values of predicted quality score 1432 and true quality score 1440.

Note that many of the subsequent graphs presented herein will have a dashed line that has a slope of 1. For purposes of this disclosure, such lines are also referred to herein as “slope 1 line” or “lines with slope 1”.

Graph 1500a of FIG. 15A has a line 1510a depicting the relationship between the predicted quality scores 1432 (X axis) and the true quality scores 1440 (Y axis), for a specific implementation of a base caller. As seen in FIG. 15A, for higher values of the predicted score 1432, the predicted score 1432 is usually more than the corresponding true score 1440. For example, a predicted quality score of 45 roughly corresponds to a true quality score 1440 of about 32. Thus, when a quality score Q is predicted by the base caller of FIG. 15A as being 45, it should empirically be around 32. Thus, the base caller is predicting quality scores that are higher than corresponding true or empirically calculated quality scores. Thus, the base caller that generates the graph 1500a of FIG. 15A is “overconfident” about the prediction of the quality scores.

Graph 1500b of FIG. 15B has a line 1510b depicting the relationship between the predicted quality score 1432 and the true quality score 1440, for another specific implementation of a base caller. As seen in FIG. 15B, the predicted score 1432 is usually less than the corresponding true score 1440. For example, a predicted quality score of 45 roughly corresponds to a true quality score 1440 of about 50. Thus, when a quality score Q is predicted by the base caller of FIG. 15B as being 45, it should empirically be around 50. Thus, the base caller is predicting quality scores that are lower than corresponding true or empirically calculated quality scores. Thus, the base caller that generates the graph 1500b of FIG. 15B is “underconfident” about the prediction of the quality scores.

Thus, as seen in FIGS. 15A and 15B, a base caller can be overconfident or underconfident when predicting the quality scores. Ideally, the quality scores predicted by the base caller should fully, or at least substantially (e.g., within a threshold of 1% or 5% or less) match the true quality scores. Note that any point in the slope 1 line (e.g., lines 1505a and 1505b of FIGS. 15A and 15B, respectively) has equal values of predicted quality score 1432 and true quality score 1440. Thus, it is desirable that the predicted quality scores versus true quality scores graph should overlap the slope 1 line, or should closely follow (or closely align to) the slope 1 line. However, as seen in FIGS. 15A and 15B, the quality scores predicted by the base caller may not always match with the true quality scores (i.e., the points on the graph may not lie on the slope 1 line), thereby resulting in not fully accurate quality scores being generated by the base caller.

### Diagonal Mismatch Region 1625 and Overconfident (or Saturation) Region 1620

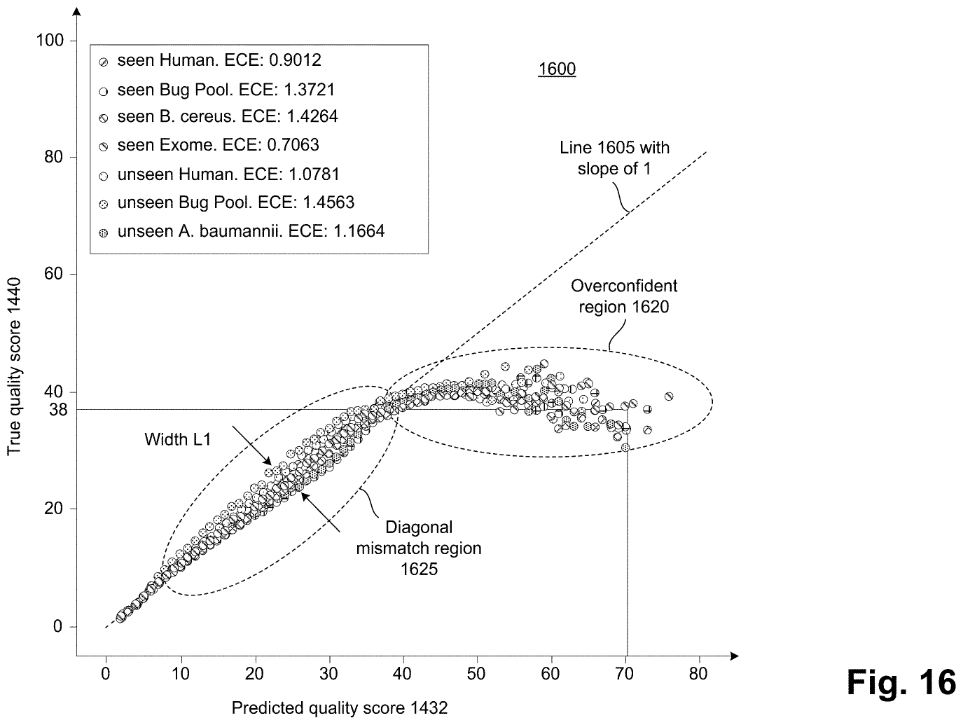

FIG. 16 illustrates another graph 1600 depicting a comparison between predicted quality scores 1432 (X axis) and true quality scores 1440 (Y axis). Similar to FIGS. 15A and 15B, the graph 1600 of FIG. 16 also includes a “slope 1” line 1605 having a slope of 1. The graph 1600 has a plurality of sampling points, for example, corresponding to human genome, and various other types of genomes, such as genomes of Acinetobacter baumannii (A. baumannii) bacteria, Bacillus cereus (B. cereus) bacteria, exomes, and bug pool genomes.

In the graph 1600 of FIG. 16, two main regions 1620 and 1625 (roughly illustrated using dotted lines) are identified, where each of the regions have mismatches between a plurality of graph sampling points and the slope 1 line. Note that as discussed, it is desirable that the graph sampling points closely overlap or align with the slope 1 line, for a close match between the predicted quality scores 132 and the true quality scores 1440.

For example, region 1625, also referred to herein as diagonal mismatch region 1625, identifies mismatch between the predicted quality score 132 and the true quality score 1440 in the diagonal region of the graph (e.g., on a region lying on the slope 1 line). In this specific example of FIG. 16, the diagonal mismatch region 1625 is mainly between true quality scores of about 15 to 40. In this region the sampling points are scattered around the slope 1 line, and many sampling points are off or misaligned with respect to the slope 1 line. For example, the substantially widest section of the region 1625 has a width of L1. Again, ideally, this width should be close to zero, with all the sampling points being close to the slope 1 line.

The region 1620 is also referred to herein as overconfident region 1620 (or saturation region 1620), as the base caller 1416 is overconfident in this region. For example, for a sampling point lying within this region 1620, a corresponding predicted quality score is higher than a corresponding true quality score. For example, the true quality scores of sampling points within this region 1620 are between about 35 and 40. However, the predicted quality score of sampling points within this region 1620 are above 40. For example, an example sampling point within this region 1620 has a predicted quality score that is as high as 70, but has a true quality score of about 38. Thus, in the region 1620, the base caller 1416 is overconfident in its quality score prediction.

As illustrated, in the overconfident region 1620, the predicted quality score saturates. That is, in the overconfident region 1620, an increase in the predicted quality score does not result in corresponding significant increase in the true quality score. Thus, the overconfident region 1620 is also referred to as saturation region.

Note that the true probability quality scores 1440 does not go above a threshold true score, which is about 40 (which translates to a probability score of 0.9999 and an error rate of 0.01%) in an example. This may be because of errors in the sequencing machine 1404 and/or the base calling system, which may occur due to amplification, preparation, bridge PCR, or other reasons. For example, during the previously discussed amplification process, amplification error may occur. For example, library preparation error may occur for preparation of input library during the amplification process. Another example of an error is associated with the bridge PCR. Such errors impose a limit on maximum achievable true quality scores. For example, due to these errors, even an adequately trained base caller may not predict quality scores that are truly above a threshold quality score. Another limitation is associated with the limited by the amount of data used. For example, each bin should have an adequate number of basecalls, to determine if the quality score is relatively well calibrated. Merely as an example, for bin Q40 (i.e., a bin including quality score of 40), there has to be at least, for example, 10,000 basecalls, but likely many more to reliably determine the error rate. This problem becomes worse for relatively higher Q scores, because the basecaller may not predict enough bases with scores that high. So, an ability to well calibrate relatively high quality-scores is also limited. The threshold quality score in the example of FIG. 16 is about 40 or 45. Thus, although the overconfident base caller predicts a quality score of 60 or 70, the true quality score is still within the threshold quality score of 40, as illustrated in FIG. 16.

### Correction of Mismatch Between True Quality Scores 1440 and Predicted Quality Scores 1432

It may be desirable for the sampling points of the graph 16A to closely follow the slope 1 line, e.g., in both regions 1620 and 1625. For example, it is desirable that the predicted quality scores 1432 closely align to the true quality scores 1440. Various approaches have been discussed herein later in turn, which may at least in part achieve this objective. Such approaches can be broadly classified in three categories:
1. Input normalization
2. Quality score remapping
3. Loss penalization

Each of these approaches is discussed in further detail herein below in turn.

### Input Normalization

FIG. 17A illustrates a base calling system 1700 including a normalization module 1704 for normalizing sensor data that are received by a base caller 1416. The base calling system 1700 of FIG. 17A is at least in part similar to the base calling system 1400 of FIG. 14A, and similar components in the two systems are labeled using the same labels. For example, similar to the base calling system 1400 of FIG. 14A, the base calling system 1700 of FIG. 17A includes the sequencing machine 1404 comprising the flow cell 1405, where the flow cell 1405 generates sensor data 1412. Also similar to the base calling system 1400 of FIG. 14A, the base calling system 1700 of FIG. 17A includes the base caller 1416 and the quality score generation module 1428.

In an embodiment, unlike the base calling system 1400 of FIG. 14A, the base calling system 1700 of FIG. 17A includes a normalization module 1704 configured to receive the sensor data 1412, normalize the sensor data 1412 to generate normalized sensor data 1712, and provide the normalized sensor data 1712 to the base caller 1416. Thus, instead of operating on the sensor data 1412 (as discussed with respect to the system 1400 of FIG. 14A), the base caller 1416 of the system 1700 of FIG. 17A now operates on the normalized sensor data 1712.

FIG. 17B illustrates two graphs 1701 and 1711 depicting a normalization operation on sensor data performed by the normalization module 1704 of the base calling system of FIG. 17A. Specifically, the first graph 1701 of FIG. 17B illustrates a histogram associated with the sensor data 1412, and the second graph 1711 of FIG. 17B illustrates another histogram associated with the normalized sensor data 1712.

Referring now to the first graph 1701 of FIG. 17B, illustrated is a histogram depicting distribution of intensity of sensor data 1412. Note that in this example, the sensor data 1412 is assumed to be images of clusters having specific intensities. However, such an assumption does not limit the scope of this disclosure. For example, the teachings of this disclosure are also applicable for other types of sensor data, such as when the sensor data are represented by electrical signals (such as voltages or currents), chemical properties (e.g., pH levels), or the like.

The image intensity in the X axis of the graph 1701 ranges from about 220 to about 820, which is labeled as a first range 1702 in the graph 1701, where the image intensity has any appropriate unit. The first range 1702, thus, is defined by a corresponding lower intensity of 220 and a corresponding upper intensity of 820. As discussed herein previously, the intensities are captured by image sensors in the flow cell, As previously discussed herein, an image intensity captured from a cluster during a sequencing cycle is indicative of a base to be called for that cluster for that sequencing cycle.

As seen in the intensity versus frequency plot of the graph 1701, most (e.g., 99.0%) of intensities are within a second intensity range 1706, where the second intensity range 1706 is between about 240 and 760. For example, intensity value 240 represents a lower 0.5^{th} percentile, where only 0.5% of intensities are below 240 and the remaining 99.5% intensities are above 240. Similarly, intensity value 820 represents an upper 99.5^{th} percentile, where 99.5% intensities are below 820 and only 0.5% intensities are above 820. That is, 99% of the intensities are between intensity range 240 to 820, which is labelled as the second range 1706 in FIG. 17B. Note that the example of 0.5% used herein is merely an example, and in other examples, another appropriate percentage (such as 0.05% or 1%) can be used. The second range 1706, thus, are defined by a lower intensity of 240 and an upper intensity of 760. As seen, the second range 1706 is fully encompassed with the first range 1702.

In an example, the intensities outside this second range 1706 are outlier intensities that may not, in some examples, help in generating predicted quality scores matching true quality scores. Put differently, the outlier intensities result in some mismatch between the predicted quality scores and the true quality scores. Accordingly, in an embodiment, these outliers are removed during the normalization process.

For example, during the normalization process, intensities that are lower than the second range 1706 (also referred to as lower outlier intensities) are assigned a value corresponding to a lower intensity of the second range 1706. Thus, in the example of FIG. 17B, the lower outlier intensities (i.e., intensities that are between 220 and 240) are assigned an intensity of 240. Note that there are mere 0.5% lower outlier intensities that are below 240 and are assigned the intensity of 240. However, in another example, instead of assigning an intensity of 240 to the lower outlier intensities, the lower outlier intensities are simply removed from consideration during the normalization process.

Similarly, during the normalization process, intensities that are higher than the second range 1706 (also referred to as higher outlier intensities) are assigned a value corresponding to an upper intensity of the second range 1706. Thus, in the example of FIG. 17B, the higher outlier intensities (i.e., intensities that are between 760 and 820) are assigned an intensity of 760. Note that there are a mere 0.5% of intensities that are above 760 and are assigned the intensity of 760. However, in another example, instead of assigning an intensity of 760 to the higher outlier intensities, the higher outlier intensities are simply removed from consideration during the normalization process.

Thus, subsequent to processing the lower outlier intensities and the higher outlier intensities (e.g., by either respectively assigning the lower and upper intensities of the second range 1706 to these outlier intensities, or by simply ignoring these outlier intensities), the intensities now are only within the second range 1706. That is, now no outlier intensities are present. Subsequently, the intensities within the second range 1706 are mapped to a third range 1722 of intensities, as illustrated in the graph 1711 of FIG. 17B.

In the example of FIG. 17B, the third range 1722 is defined by a lower intensity of 0 and an upper intensity of 255. Thus, intensities within the third range 1722 can be represented using 8-bit data. In other examples, other upper and lower intensities for the third range 1722 can be used.

In an example, the third range is less than the second range. For example, the second range is from intensity 240 to 760, i.e., an intensity range of 520. In contrast, the third range is from intensity 0 to 255, i.e., an intensity range of 256. That is, the intensities in the second range are squeezed and mapped to the third range.

During the mapping process, a sensor data having a first intensity value in the second range 1706 is mapped to have a second intensity value in the third range 1722. For example, the second range is defined by intensities 240 and 760—i.e., has an intensity range of (760-240)=520. The third range is defined by intensities 0 and 255—i.e., has an intensity range of 256. Thus, merely as examples, intensities between 240 and 242 in the second range 1706 are mapped to intensity 0 in the third range 1722; intensities between 242 and 244 in the second range 1706 are mapped to intensity 1 in the third range 1722; intensities between 758 and 760 in the second range 1706 are mapped to intensity 255 in the third range 1722, and so on. Thus, the two histograms in the graphs 1701 and 1711 have somewhat same shape. In an example, a sum of all the bars in the histogram of graph 1701 and a sum of all the bars in the histogram of graph 1701 are substantially the same. In an example, an area covered under the first histogram (associated with the graph 1701) within the second range 1706 and an area covered under the second histogram (associated with the graph 1711) within the third range 1722 are substantially equal.

The normalization, which includes processing the outlier intensities and the mapping, lowers variability between images from different sequencing runs and different sequencing run preparation process, and knowledge is more transferrable between images of the sensor data.

### Normalization Results

FIG. 17C illustrates a graph 1710 depicting a comparison between predicted quality scores 1432 and true quality scores 1440, wherein the sensor data 1412 have been normalized by the normalization module 1704 of the base calling system 1700 of FIG. 17A while generating data for the graph of FIG. 17C. Similar to FIG. 16, the graph 1710 of FIG. 17C also includes a “slope 1” line 1785 having a slope of 1. The graph 1710 has a plurality of sampling points for, for example, human genome, and various other types of genomes, such as genomes of Acinetobacter baumannii (A. baumannii) bacteria, Bacillus cereus (B. cereus) bacteria, exomes, and bug pool genomes, e.g., similar to the graph 1600 of FIG. 16.

Thus, the graph 1600 of FIG. 16 is generated by a base calling system that does not normalize the sensor data 1412 (e.g., the base calling system 1400 of FIG. 14A), whereas the graph 1710 of FIG. 17C is generated by a base calling system that normalizes the sensor data 1412 and uses the normalized sensor data for base calling (e.g., the base calling system 1700 of FIG. 17A).

Comparing the overconfident region 1620 of the graph 1600 of FIG. 16 and a similar overconfident region 1720 of the graph 1710 of FIG. 17C, it is seen that there is no substantial change in the overconfident regions of the two graphs. That is, the normalization process may not significantly contribute to improving performance in the overconfident region 1720.

Comparing the diagonal mismatch region 1625 of the graph 1600 of FIG. 16 and a similar diagonal mismatch region 1725 of the graph 1710 of FIG. 17C, significant performance improvement is noticed. For example, as discussed previously, the diagonal mismatch region identifies mismatch between the predicted quality score 132 and the true quality score 1440 in the diagonal region of the graph (e.g., on a region lying on the slope 1 line). The diagonal mismatch region is mainly between true quality scores of 15 to 40. In this region the sampling points are scattered around the slope 1 line, and many sampling points are off the slope 1 line.

For example, the substantially widest section of the region 1625 in the graph 1600 of FIG. 16 has a width of L1 Again, ideally, this width should be close to zero, with all the sampling points being close to the slope 1 line.

A corresponding substantially widest section of the region 1725 in the graph 1710 of FIG. 17C has a width of L2. As seen, L2 in FIG. 17C is substantially lower than L1 in FIG. 16 (i.e., L2<L1). That is, in the graph 1710 of FIG. 17C, due to the normalization process, the sampling points are less scattered and better aligned to the slope 1 line, e.g., compared to the scattering and alignment of the sampling points in the graph 1600 of FIG. 16. Thus, the inventors of this disclosure have found that, for true quality score between about 15 and 40, the normalization process helps the predicted quality score 1432 be better aligned to the true quality score 1440 (e.g., compared to a scenario without normalization).

FIG. 17D illustrates a plot indicating expected calibration error (ECE) for a base calling system having input normalization versus another base calling system lacking such an input normalization. As seen, input normalization improves ECE for most types of genomes experimented upon by the inventors.

FIG. 17E illustrates a color comparison between the sensor data 1412 prior to normalization and normalized sensor data 1712. For example, a first image 1790a illustrates sensor data 1412 captured from the flow cell, prior to any normalization Locations of fiducials are illustrated in the image 1790a using oval shapes. A solid support upon which a biological specimen is imaged can include such fiducial markers, to facilitate determination of the orientation of the specimen or the image thereof in relation to probes that are attached to the solid support. Exemplary fiducials include, but are not limited to beads (with or without fluorescent moieties or moieties such as nucleic acids to which labeled probes can be bound), fluorescent molecules attached at known or determinable features, or structures that combine morphological shapes with fluorescent moieties. Exemplary fiducials are set forth in U.S. Patent Publication No. 2002/0150909, which is incorporated herein by reference. Multiple (such as hundreds of thousands, or even millions) of clusters, although not labelled, are included in the illustration of FIG. 17E. Image data on and around a cluster is to be analyzed, to make a base call for the cluster. Note that the intensity scale in image 1790a is from 0 to 2000, with intensities around 200 to 800 being predominantly present, as discussed with respect to FIG. 17B.

A second image 1790b illustrates normalized sensor data 1712, e.g., after normalization has been performed on the sensor data 1412. Locations of the clusters are illustrated in the image 1790a using oval shapes. Image data on and around a cluster is to be analyzed, to make a base call for the cluster. Note that the intensity scale in image 1790b is from 0 to 255, e.g., as a result of normalization

### Normalization Method

FIG. 17F illustrates a flowchart depicting an example method 1750 for normalizing sensor data, and using normalized sensor data for base calling operations.

At 1755 of the method 1750, a plurality of sensor data is received (e.g., by the normalization module 1704 of FIG. 17A) from a flow cell, where the plurality of sensor data are within a first range (e.g., first range 1702). For example, FIG. 17B illustrates an example in which the plurality of sensor data comprises the plurality of intensity values that are within the first range 1702.

At 1760, a second range is identified (e.g., by the normalization module 1704 of FIG. 17A), such that at least a threshold percentage of the plurality of sensor data are within the second range. For example, FIG. 17B illustrates an example of the second range 1706, such that 99.0% of the sensor data are within this range. Note that 99.0% is used merely as an example, and other threshold percentages can also be envisioned by those skilled in the art, based on the teachings of this disclosure.

At 1765, the outlier sensor data, e.g., sensor data that are outside the second range, are processed (e.g., by the normalization module 1704 of FIG. 17A). As discussed herein previously, in one example, the lower outlier sensor data (e.g., intensities that are between 220 and 240 in FIG. 17B) are assigned an intensity corresponding to a lowest value (e.g., 240) of the second range, as discussed with respect to FIG. 17B. Similarly, in one example, the upper outlier sensor data (e.g., intensities that are between 760 and 820 in FIG. 17B) are assigned an intensity corresponding to a highest value (e.g., 760) of the second range, as also discussed with respect to FIG. 17B. In another example, the outlier sensor data are simply ignored or taken out of consideration.

At 1770, at least a subset of the plurality of sensor data, e.g., which are within the second range, are mapped to a third range (e.g., by the normalization module 1704 of FIG. 17A), to generate a plurality of normalized sensor data 1770. For example, as illustrated in FIG. 17B, intensities in the second range in the graph 1701 are mapped to corresponding intensities in the third range in the graph 1711. In an example, if the outlier sensor data are taken out of consideration, then such outlier sensor data are not mapped at 1770, and only a subset of the plurality of sensor data, which are in the second range, are mapped to the third range.

At 1775, the plurality of normalized sensor data is processed in a base caller, to call, for each of the plurality of normalized sensor data, a corresponding base. For example, the base caller 1416 of FIG. 17A receives the normalized sensor data 1712, and generates corresponding base calls.

### Quality Score Remapping and Quantization

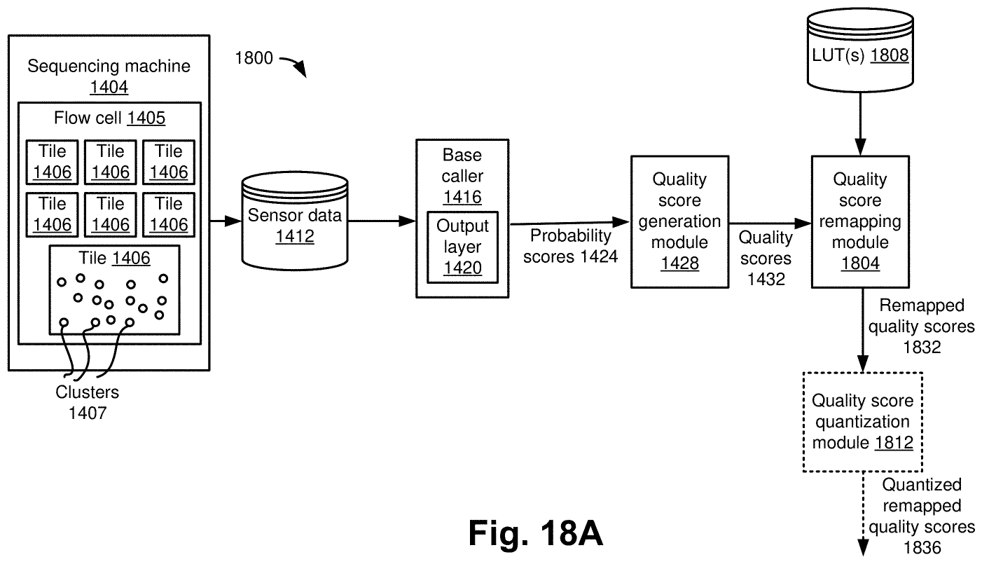

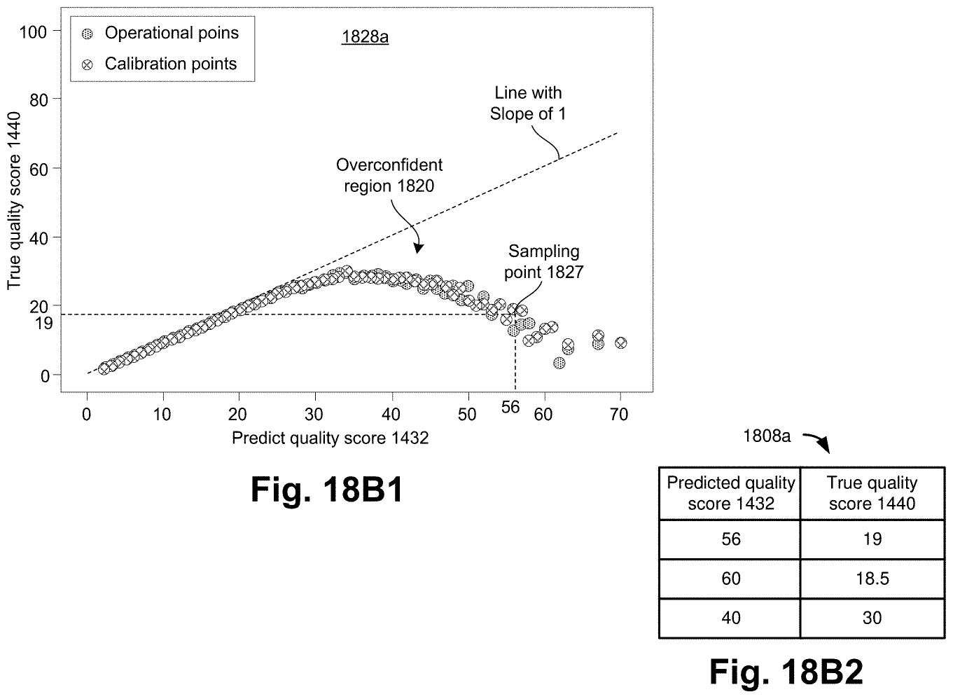

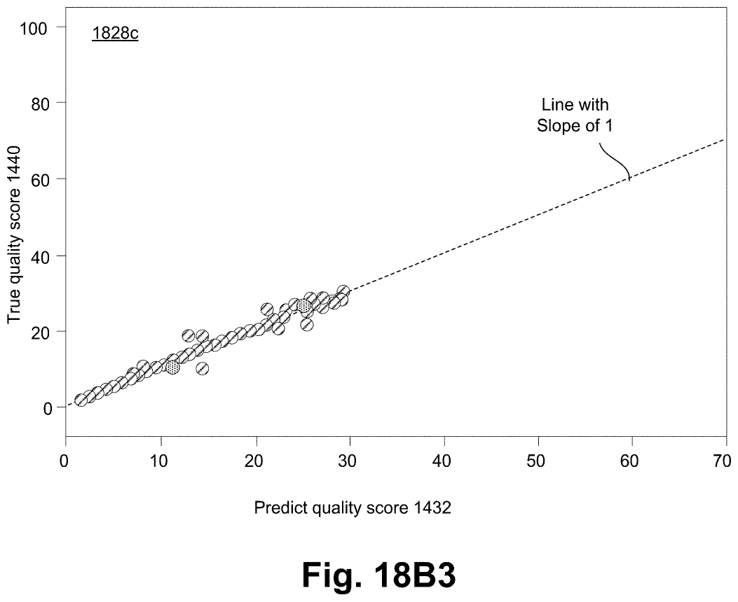

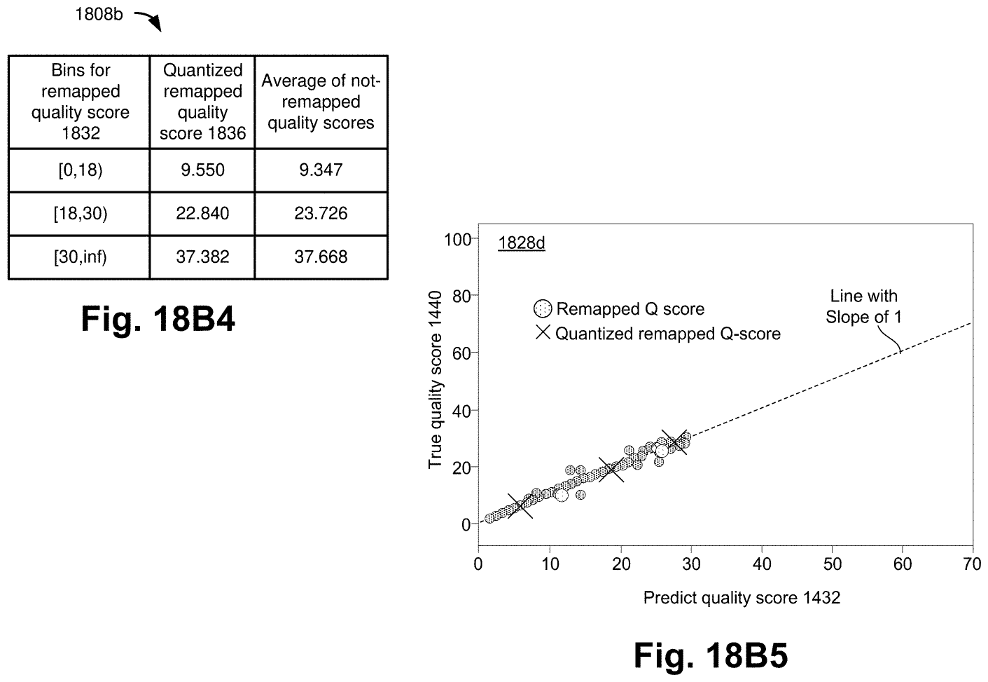

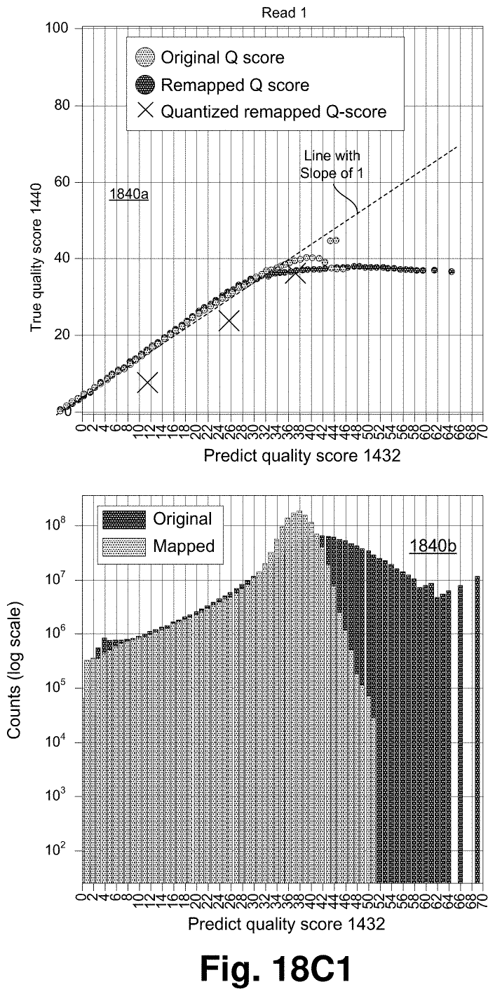

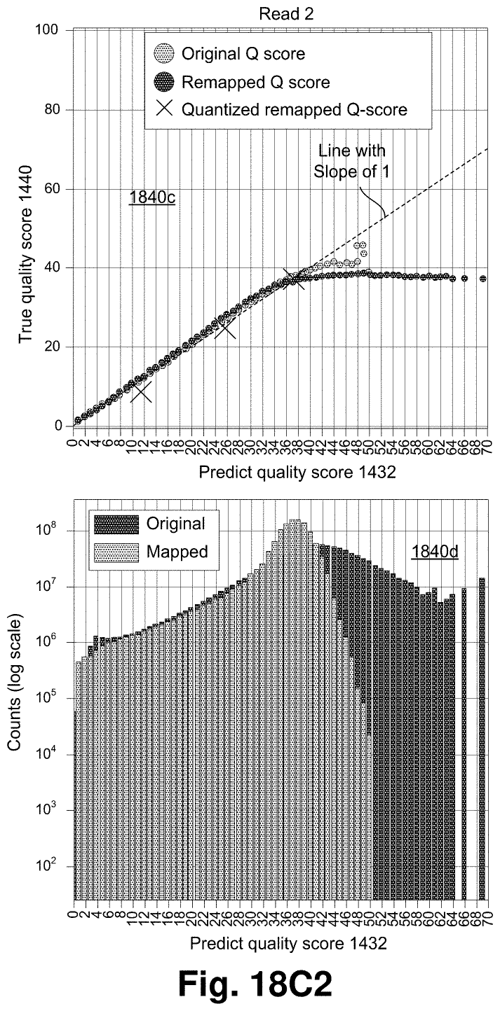

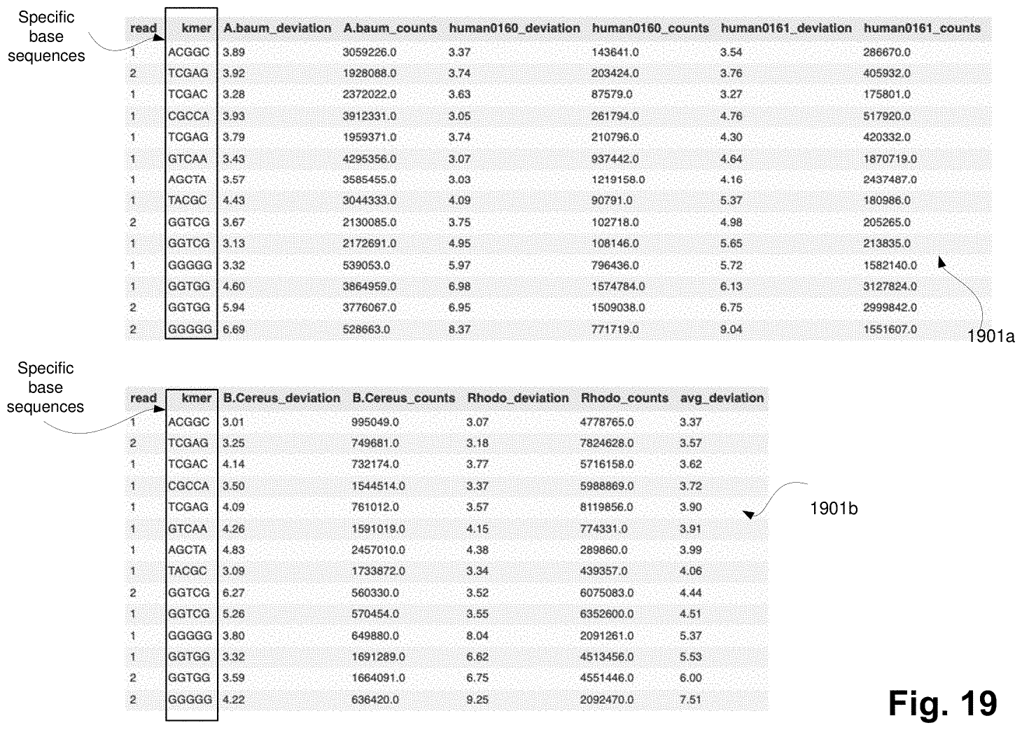

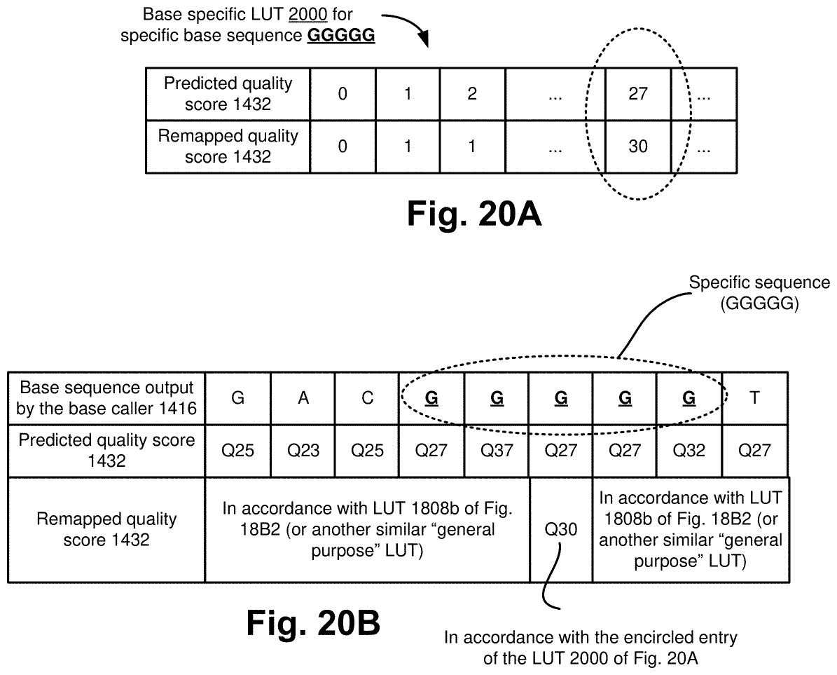

FIG. 18A illustrates a base calling system 1800 including a quality score remapping module 1804 for selectively remapping quality scores 1432 predicted by the base caller 1416. The base calling system 1800 of FIG. 18A is at least in part similar to the base calling system 1400 of FIG. 14A, and similar components in the two systems are labelled using the same labels. For example, similar to the base calling system 1400 of FIG. 14A, the base calling system 1800 of FIG. 18A includes the sequencing machine 1404 comprising the flow cell 1405, where the flow cell 1405 generates sensor data 1412. Also similar to the base calling system 1400 of FIG. 14A, the base calling system 1800 of FIG. 18A includes the base caller 1416 and the quality score generation module 1428.

Although not illustrated, in an example, the system 1800 of FIG. 18A may include the normalization module 1704 of FIG. 17A. In such an example, the base caller 1416 operates on the normalized sensor data 1712. However, in another example, the system 1800 of FIG. 18A lacks such a normalization module 1704.

In an embodiment, unlike the base calling system 1400 of FIG. 14A, the base calling system 1800 of FIG. 18A includes a quality score remapping module 1804 configured to selectively remap the quality scores 1432 generated by the quality score generation module 1428, as discussed herein below.

In an embodiment, in addition to remapping the quality scores, the base calling system 1800 also may include a quality score quantization module 1812 that quantizes the remapped quality scores 1832, to generate quantized remapped quality scores 1836. In an example, the quality score quantization module 1812 is optional, and hence, is illustrated using dashed lines in FIG. 18A. In an embodiment, the system 1800 further comprises one or more Look Up Table(s) (LUTs) 1808 stored in a memory that is accessible to the quality score remapping module 1804.

### Quality Score Remapping and Quantization Examples

FIGS. 18B1, 18B2, 18B3, 18B4, and 18B5, in combination, illustrate examples of quality score remapping and quantization. Referring to FIG. 18B1, illustrated is a graph 1828a depicting predicted quality scores 1432 output by the base caller 1416 in the X axis, and corresponding true quality scores 1440 in the Y axis. As discussed with respect to FIG. 16, in an overconfident region 1820 (see FIG. 16 for further detail), the predicted quality score is higher than corresponding true score.

For example, a sampling point 1827 (in the overconfident region 1820) corresponding to a specific base of a specific cluster has a predicted quality score of 56 and a true quality score of 19. Accordingly, in an example, the remapping module 1804 maps a quality score 1432 having a value of 56 to a remapped quality score having a value of 19.

Note that the graph 1828a includes two types of sampling points: calibration points and operational points. The calibration points have known ground truth base calls and known true quality scores 1440. The calibration points are used to generate a LUT for the remapping (see FIG. 18B2), and subsequently the operational points use the LUT for being remapped to new quality scores. The assumption here is that the remapping LUT generated using the calibration points is applicable for the operational points as well.

Now referring to FIG. 18B2, illustrated is an example remapping LUT 1808a that stores mapping data between predicted quality scores 1432 and true quality scores 1440. For example, as discussed with respect to FIG. 18B1, a predicted quality score of 56 actually corresponds to a true quality score of 19, as indicated in a first row of the remapping LUT 1808a. Other rows of the remapping LUT 1808a are similarly populated.

Note that the LUT 1808a is an oversimplified remapping LUT, to illustrate the teachings of this disclosure. In a real-life implementation, a remapping LUT is likely to have many more rows, for remapping various predicted quality scores 1432 to corresponding true quality scores 1440.

Referring to FIG. 18B3, illustrated is a graph 1828c depicting remapped quality scores for the operational points of the graph 1828a of FIG. 18B1. As illustrated in FIG. 18B3, after the quality scores are remapped, the sampling points corresponding to the quality scores now better align with the line with slope 1 (e.g., relative to the alignment of FIG. 18B1). Thus, the remapped quality scores of FIG. 18B3 are now substantially closer to (equal to) their respective true quality scores (e.g., relative to the alignment of FIG. 18B1). Note that the remapping helps in alignment in the overconfident region 1820.

FIG. 18B4 illustrates a LUT 1808b for quantizing the remapped quality scores. In the example of FIG. 18B4, each remapped quality score is assigned to one of 3 quantized quality scores corresponding to the three rows of the LUT 1808b. However, such a number of quantized quality scores is a mere example and does not limit the scope of this disclosure. For example, in another example, each remapped quality score can be assigned to one of Q number of quantized quality scores corresponding to Q number of rows of a LUT, where Q can be two, four, higher.

In the example of FIG. 18B4, the remapped quality scores are assigned or grouped into three bins [0,18), [18,30), and [30,infinite) (see first column of the LUT 1808b), although the ranges of the bins are mere examples and does not limit the scope of this disclosure. The second column of the LUT 1808b indicates example quantized remapped quality scores corresponding to each bin. For example, remapped quality scores included in the bin [0,18) are assigned a quantized remapped quality score of 9.550; remapped quality scores included in the bin [18,30) are assigned a quantized remapped quality score of 22.840; and remapped quality scores included in the bin [30,inf) are assigned a quantized remapped quality score of 37.382.

The quantized quality scores 9.550, 22.840, and 37.382 are pre-specified in the LUT. In an example, these numbers are generated by averaging the true quality scores of calibration sampling points (see FIG. 18B1) assigned to corresponding bins. For example, assume that 300 calibration sampling points are assigned to the bin [0,18). An average of the true quality scores of these 300 calibration sampling points, which are assigned to the bin [0,18), is determined to be 9.550. Accordingly, the bin [0,18) is assigned remapped quantized quality score of 9.550, which is an average of true quality scores of calibration sampling points included in this bin.

The third column of the LUT 1808b indicates an average of original (i.e., not remapped) quality scores in the respectively bin. For example, following on with the above example where the 300 calibration sampling points are assigned to the bin [0,18), an average of their quality scores prior to the remapping is 9.347. Thus, by comparing the second and third columns of the LUT, one can apprehend how much the remapping changes or deviates the quality score. Put differently, for a given row (i.e., a given quality score bin), a deviation between the second and third columns of the LUT is an indication of a change in the average quality scores due to the remapping.

FIG. 18B5 is a graph 1828d illustrating the quantized scores. For example, FIG. 18B5 is at least in part similar to the graph 1828c of FIG. 18B3. However, unlike the graph 1828c of FIG. 18B3, in the graph of FIG. 18B5, the three quantized scores of the LUT 18B4 are illustrated, along with the remapped quality scores. Thus, in an example, the system 1800 outputs the quantized remapped quality scores 1836 (e.g., instead of the remapped quality scores).

FIG. 18C illustrates two further examples of quality score remapping and quantization. For example, quality score remapping and quantization for sequencing read cycle 1 (referred to as Read 1) and sequencing read cycle 2 (referred to as Read 2) are illustrated.

Referring to the example of Read 1, two graphs are illustrated under Read 1: (i) a top graph 1840a illustrating remapping and quantization, and (ii) a bottom graph 1840b which is a histogram. For example, in the graph 1840a, as seen in red colored sampling dots, quality scores above about 40 deviate away from the line with slope 1. The quality scores are remapped, which are illustrated using blue dots. As seen, the remapped quality scores are better aligned with the slope 1 line (e.g., relative to the quality scores before remapping). The histogram 1840b illustrates the original quality scores in red, and the remapped quality scores in blue. As illustrated, the original scores can be as high as 65 or 70, whereas the remapped quality scores are less than about 52.

Referring now to the example of Read 2, two graphs are illustrated under Read 2: (i) a top graph 1840c illustrating remapping and quantization, and (ii) a bottom graph 1840d which is a histogram, each of which will be evident based on the above discussion with respect to the graphs of Read 1.

### Quality Score Remapping and Quantization for Specific Base Sequences

In some implementations, the base caller 1416 makes a base call for a current sequencing cycle by processing a window of sequencing images for a plurality of sequencing cycles, including the current sequencing cycle contextualized by right and left sequencing cycles. In an example, the base “G” is indicated by a dark or off state in the sequencing images. Accordingly, in an example, repeat patterns of the base “G” can lead to higher likelihood of erroneous base calls. Such erroneous base calls may also occur when the current sequencing cycle is for a non-G base (e.g., base “T”), but right and left flanked by Gs.

In an example, there are some specific base calling sequence patterns for which the probability of error in base calling is relatively high. For example, for base sequences of homopolymers (e.g., GGGGG) or flanked-homopolymers (e.g., GGTGG), the probability of error in base calling is relatively high. There may be other specific base calling sequence patterns, such as GGTCG, for which probability of error in base calling is also relatively high. In an example, such specific base calling sequence patterns have multiple G's, such as G's at least at a beginning and at an end of the sequence, and possibly a third G between the two end-G's in the 5-base sequence. Other examples of such specific base calling sequences include GGXGG, GXGGG, GGGXG, GXXGG, and GGXXG, where X can be any of A, C, T, or G.

FIG. 19 illustrates a table depicting, for some specific base sequences, deviations between (i) an average of quality scores of the specific base sequences and (ii) an average of remapped quality scores of the specific base sequences, where the remapping is performed in accordance with a general LUT of, for example, FIG. 18B2. Note that the table of FIG. 19 is divided in two sections 1901a and 1901b due to limitations in space for depicting the undivided table. The specific sequences depicted in the table are ACGGC, TCGAG, and so on, and finally GGGGG, GGTGG, and so on. Deviations for a read sequence 1 and read sequence 2 for various specific base sequences are illustrated. Various types of genomes are used, such as Acinetobacter baumannii (A baumannii) bacteria, human genome, Bacillus cereus (B. cereus) bacteria, and Rhodobacter. For each type of genome, a corresponding count of base sequences and a corresponding deviation is used. Finally, an average deviation for the various specific base sequences are listed in the last column of the section 1901b of the table of FIG. 19. The deviations presented in FIG. 19 represents an amount by which average quality scores change due to the remapping process, when a generate purpose LUT (such as in FIG. 18B2) is used for the remapping.

Referring to the second column (i.e., Specific base sequences) and the last column (i.e., average deviation) of the section 1901b of FIG. 19, it is seen that for certain specific base sequences, the deviations for at least some of the specific base sequences are significant. For example, the average deviation for read 2 of GGGGG is 7.51 and the average deviation for read 2 of GGTGG is 6, which are significant (e.g., compared to an average deviation of 3.37 for read 1 of ACGGC). Thus, a remapping that works for general base sequences may not adequately work for at least some of the specific base sequences.

FIG. 20A illustrates a LUT 2000 that is usable to remap predicted quality scores of a specific base sequence (e.g., a homopolymer sequence of GGGGG) to remapped true quality scores. Note that the LUT 2000 is specifically for the homopolymer sequence of GGGGG, which can be derived by repeatedly testing with the homopolymer sequence of GGGGG, and generating true quality scores for the predicted base sequence. More specifically, the LUT 2000 is for remapping predicted quality score of a middle G of the sequence of GGGGG. For example, referring to an encircled entry of the LUT 2000, a predicted quality score of 27 can be remapped to a true quality score of 30 for a middle G of the specific sequence of GGGGG.

FIG. 20B illustrates remapping of predicted quality scores for a specific base sequence (e.g., a homopolymer sequence of GGGGG) using the LUT 2000 of FIG. 20A. For example, in FIG. 20B, a base sequence of G, A, C, G, G, G, G, G, T is output by the base caller, along with corresponding predicted respective quality scores of Q25, Q23, Q25, Q27, Q37, Q27, Q27, Q32, and Q27 for individual bases on the precited sequence, as illustrated in the first two rows of the table of FIG. 20B. That is, the first G in the sequence is associated with a predicted quality score of 25, the second A in the sequence is associated with a predicted quality score of 23, and so on. Note the presence of the specific homopolymer sequence of GGGGG in the base calls.

As illustrated in FIG. 20B, the predicted quality scores for all the bases, except for the middle G of the homopolymer sequence of GGGGG, are remapped using the LUT 1808b of FIG. 18B2 (or another similar “general purpose” LUT). Note that the LUT 1808b of FIG. 18B2 is referred to herein as a “general purpose” remapping LUT, as this LUT is used to remap general base sequences.

In contrast, the LUT 2000 of FIG. 20A is a “base sequence specific” LUT that is dedicated specifically to a middle base of a specific base sequence of GGGGG. Thus, the predicted quality middle Q27 of the middle G of this sequence in FIG. 20B is replaced in accordance with the dotted encircled entry the LUT 2000.

Note that the 4^{th} base of G, the 6^{th} base of G, and the 9^{th} base of T in the sequence of FIG. 20B each has a quality score of Q27. The quality score of Q27 for the 4^{th} base of G and the 9^{th} base of T may be remapped similarly, e.g., using a general purpose LUT, whereas the 6^{th} base of G (which is a middle one of the specific base sequence) will be remapped differently, e.g., using a base sequence specific LUT. Thus, although all the three bases have the same quality scores of Q27, merely as an example, the 4^{th} base of G and the 9^{th} base of T may be remapped to a remapped quality score of Q32 in accordance with the general purpose LUT, whereas the 6^{th} base of G (which is a middle one of the specific base sequence) may be remapped to Q30 in accordance with the base sequence specific LUT 2000 of FIG. 20A.

FIGS. 20A and 20B are directed to the specific homopolymer sequence GGGGG Similar specific LUTs can be generated for other specific homopolymer or flanked-homopolymer sequences, such as GGTGG, GGTCG, GGXGG, GXGGG, GGGXG, GXXGG, GGXXG, or the like, where X can be any of A, C, T, or G.

Loss Penalization

FIG. 21 illustrates a base calling system 2100 that includes a loss penalization module 2106 to selectively penalize loss for one or more specific base sequences. The base calling system 2100 of FIG. 21 is at least in part similar to the base calling system 1400 of FIG. 14A, and similar components in the two systems are labelled using the same labels. For example, similar to the base calling system 1400 of FIG. 14A, the base calling system 2100 of FIG. 21 includes the sequencing machine 1404 comprising the flow cell 1405, where the flow cell 1405 generates sensor data 1412. Also similar to the base calling system 1400 of FIG. 14A, the base calling system 2100 of FIG. 21 includes the base caller 1416 and the quality score generation module 1428.

In an embodiment and as illustrated in FIG. 21, the base caller 1416 includes a forward pass section 2108, a backpropagation pass section 2112, a loss generation module 2104, and a loss penalization module 2106 of a neural network model. The loss generation module 2104 receives an output of the forward pass section (e.g., predicted base calls) and a ground truth (e.g., ground truth base sequences), and generates a loss function 2109 based on a comparison of the output of the forward pass section 2108 and the ground truth 2105. The loss penalization module 2106 penalizes the loss function 2109, to generate penalized loss function 2111. In an embodiment, the penalized loss function 2111 is used by the backpropagation section 2112 for generating input gradients and/or weight gradients, which are in turn used for adapting weights of the neural network model and thereby training the neural network model. The loss penalization module 2106 selectively penalizes the loss function 2109, e.g., if a specific base sequence (e.g., a homopolymer or a flanked-homopolymer, such as GGXGG, where X is any of A, C, T, or G) is detected.

For example, a goal of training deep neural networks (such as the neural network model of the base caller 1416) is optimization of the weight parameters in each layer of the forward pass, which gradually combines simpler features into complex features so that the most suitable hierarchical representations can be learned from data. A single cycle of the optimization process is organized as follows. First, given a training dataset, the forward pass section sequentially computes the output in each layer and propagates the function signals forward through the network. In the final layer of the forward pass section, an objective loss function (e.g., generated by the loss generation module 2104) measures error between the inferred outputs and the given labels. The loss penalization module 2106 penalizes the loss function 2109, to generate penalized loss function 2111. To minimize the training error, the backpropagation pass uses the chain rule to backpropagate error signals (e g., the penalized loss function 2111) and compute gradients with respect to all weights throughout the neural network. Finally, the weight parameters are updated using optimization algorithms based on gradient descent. Whereas batch gradient descent performs parameter updates for each complete dataset, stochastic gradient descent provides stochastic approximations by performing the updates for each small set of data examples. Several optimization algorithms stem from stochastic gradient descent. For example, a training algorithm may perform stochastic gradient descent while adaptively modifying learning rates based on update frequency and moments of the gradients for each parameter, respectively. A loss function generated by the loss generation module 2104 can be of any appropriate type, such as logistic regression/log loss, multi-class cross-entropy/softmax loss, binary cross-entropy loss, mean-squared error loss, L1 loss, L2 loss, smooth L1 loss, and Huber loss. Base callers including neural network models, which comprise a forward pass section, a backpropagation section, and a loss generation module have been discussed in further detail in U.S. Nonprovisional patent application Ser. No. 16/826,134, titled “ARTIFICIAL INTELLIGENCE-BASED QUALITY SCORING,” filed 20 Mar. 2020 (Attorney Docket No. ILLM 1008-19/IP-1747-US), which is incorporated by reference herein.

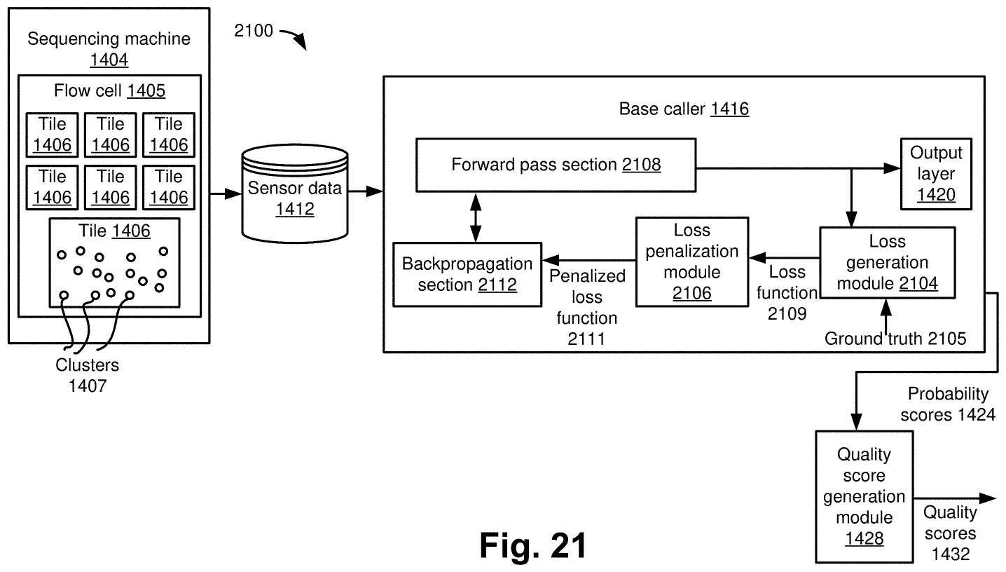

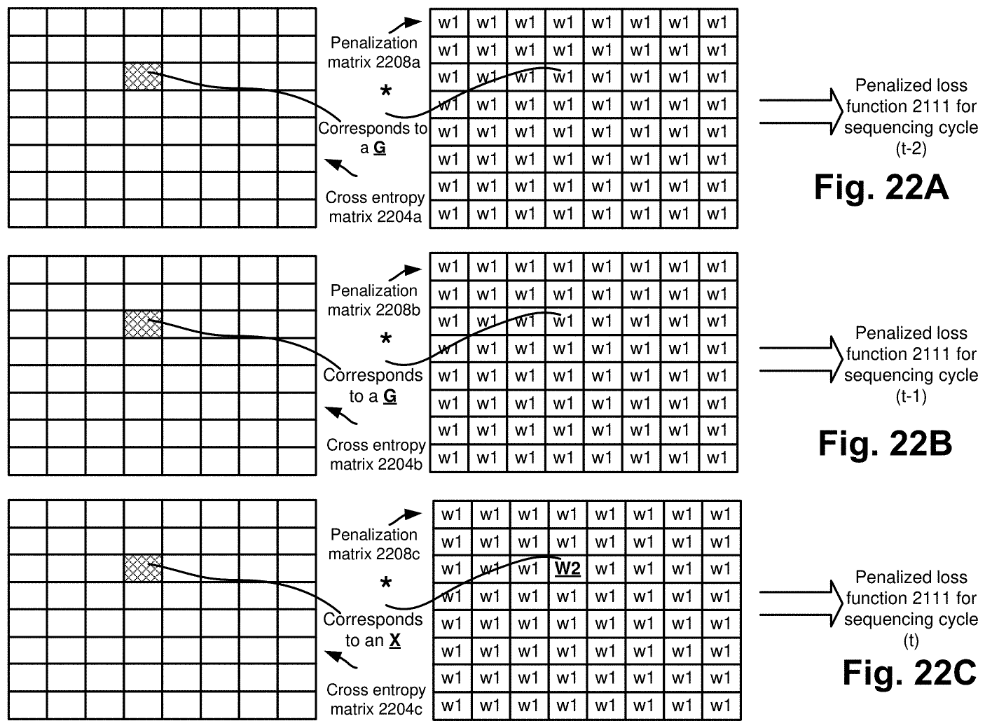

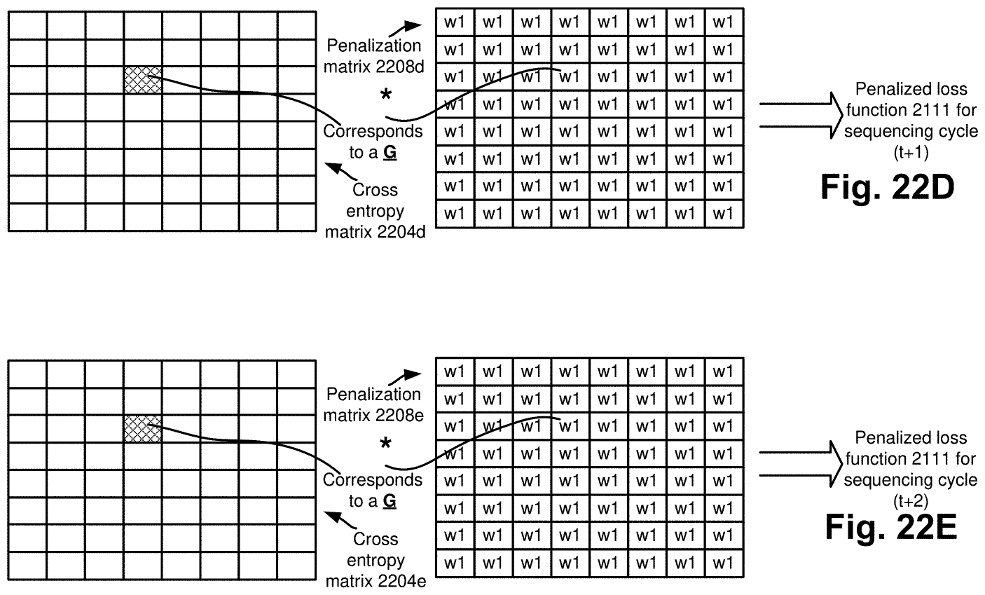

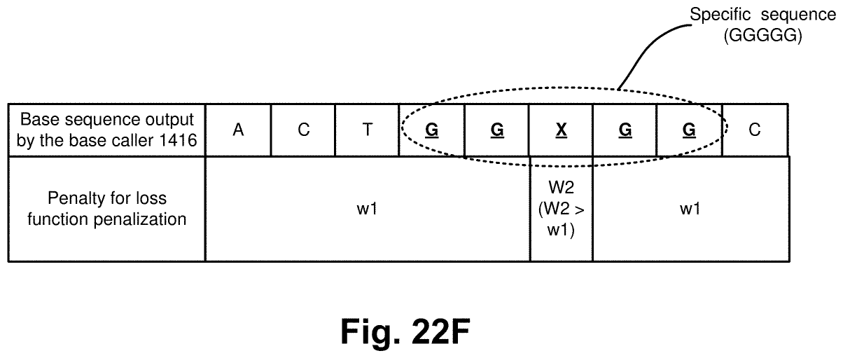

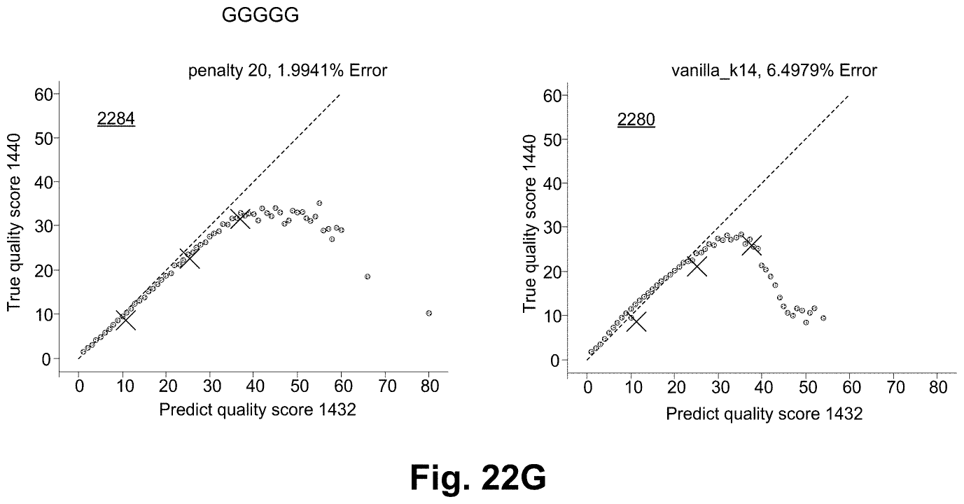

FIGS. 22A, 22B, 22C, 22D and 22E, in combination, illustrate penalization of a loss function (e.g., by the loss penalization module 2106), in response to a detection of a specific base sequence. The specific base sequence discussed with respect to the example of FIGS. 22A, 22B, 22C, 22D and 22E is GGXGG, where “X” can be any of A, C, T, or G. However, the teachings of this disclosure are not limited to any specific “specific base sequence,” and can be applied to any homopolymers, flanked homopolymers, and/or any other specific base sequence discussed herein with respect to FIGS. 19, 20A, and 20B.

Referring to FIG. 22A, illustrated is a section of a cross entropy matrix 2204a, which is a loss matrix of the loss function 2109. Also illustrated is a penalization matrix 2208a. In an example, the penalization matrix 2208a is to selectively penalize the loss function of the cross-entropy matrix 2204a. The cross-entropy matrix 2204a and penalization matrix 2208a of FIG. 22A are for a sequencing cycle (t−2). Note that each of the cross-entropy matrix 2204a and penalization matrix 2208a has multiple elements arranged in an array form, and corresponds to pixels (or subpixels) of one or more images generated for the various clusters from the flow cell.

In an embodiment, element wise multiplication of the cross-entropy matrix 2204a and penalization matrix 2208a is performed. For example, an element at position (1,1) of the cross-entropy matrix 2204a is multiplied to an element at position (1,1) of the penalization matrix 2208a; an element at position (1,2) of the cross-entropy matrix 2204a is multiplied to an element at position (1,2) of the penalization matrix 2208a; and generally speaking, an element at position (i,j) of the cross-entropy matrix 2204a is multiplied to an element at position (i,j) of the penalization matrix 2208a. In FIG. 22A, such multiplication of the cross-entropy matrix 2204a and penalization matrix 2208a generates the penalized loss function 2111 for sequencing cycle (t−2).

In general, each of the elements of the penalization matrix 2208a has a weight or penalty of w1, which may be, for example, 1. Thus, if w1=1 for an element of the penalization matrix 2208a, then the element of the penalization matrix 2208a does not impose a penalty (or imposes a penalty of 1) to a corresponding element of the cross-entropy matrix 2204a. In the example of FIG. 22A, all elements of the penalization matrix 2208a has an equal penalty of w1=1, and hence, the penalized loss function 2111 generated by multiplication of the cross-entropy matrix 2204a and the penalization matrix 2208a is merely the cross-entropy matrix 2204a. Thus, in essence, the penalization matrix 2208a does not impose a penalty in FIG. 22A.

Illustrated are a checkered entry in the cross-entropy matrix 2204a and a corresponding weight w1 in the weight matrix 2208a. Assume, for the sequencing cycle (t−2) of FIG. 22A, the ground truth base corresponding to the checkered box is a G.

Referring now to FIG. 22B, illustrated are a section of a cross entropy matrix 2204b, which is a loss matrix of the loss function 2109, and a penalization matrix 2208b for a sequencing cycle (t−1). Also illustrated is a checkered entry in the cross-entropy matrix 2204b. Also assume, for the sequencing cycle (t−1) of FIG. 22B, the base corresponding to the checkered box has a ground truth of G Again, all entries of the penalization matrix 2208b is w1=1, and hence, in effect, the penalization matrix 2208b does not impose a penalty in FIG. 22B.

Referring now to FIG. 22C, illustrated are a section of a cross entropy matrix 2204c, which is a loss matrix of the loss function 2109, and a penalization matrix 2208c for a sequencing cycle (t−1). Also illustrated is a checkered entry in the cross-entropy matrix 2204c. Also assume, for the sequencing cycle (t) of FIG. 22C, the base corresponding to the checkered box has a ground truth of X, where X can be any of A, C, T, or G. Also assume, for the sequencing cycle (t+1) of FIG. 22D, the base corresponding to the checkered box has a ground truth of G; and assume, for the sequencing cycle (t+2) of FIG. 22E, the base corresponding to the checkered box has a ground truth of G. Thus, the (3,4) position of the cross-entropy matrices 2204a, 2204b, 2204c, 2204d, and 2204e of FIGS. 22A, 22B, 22C, 22D and 22E, respectively, are associated with a specific base sequence of GGXGG. Accordingly, a middle base of this specific base sequence is penalized by the corresponding penalization matrix 2208c.

For example, a penalty corresponding to the (3,4) position of the penalization matrix 2208c of FIG. 22C, which is to be multiplied by the loss associated with the middle X of the specific base sequence (i.e., multiplied by the (3,4) element of the cross entropy matrix 2204c), is W2, where W2 is greater than w1 (i.e., W2>w1). For example, W2 is at least twice the value of w1. For example, W2 is greater than 2, whereas w1 is 1. In an example, W2=20 or higher. Remaining elements of the penalization matrix 2208c are still w1.

Hence, in effect, the penalization matrix 2208c does not impose a penalty to any of the elements of the cross-entropy matrix 2204c in FIG. 22C, except for the (3,4) element of the cross-entropy matrix 2204c that is penalized by the weight W2.

Referring now to FIG. 22D, illustrated are a section of a cross entropy matrix 2204d, which is a loss matrix of the loss function 2109, and a penalization matrix 2208d for a sequencing cycle (t+1). Also illustrated is a checkered entry in the cross-entropy matrix 2204d. As discussed previously, assume, for the sequencing cycle (t+1) of FIG. 22D, the base corresponding to the checkered box has a ground truth of G Again, all entries of the penalization matrix 2208d is w1=1, and hence, in effect, the penalization matrix 2208d does not impose a penalty in FIG. 22D.

Referring now to FIG. 22E, illustrated are a section of a cross entropy matrix 2204e, which is a loss matrix of the loss function 2109, and a penalization matrix 2208e for a sequencing cycle (t+2). Also illustrated is a checkered entry in the cross-entropy matrix 2204e. As discussed previously, assume, for the sequencing cycle (t+2) of FIG. 22E, the base corresponding to the checkered box has a ground truth of G Again, all entries of the penalization matrix 2208e is w1=1, and hence, in effect, the penalization matrix 2208e does not impose a penalty in FIG. 22E.

Thus, in the five consecutive base calling cycles illustrated in FIGS. 22A, 22B, 22C, 22D and 22E, the checkered boxes are associated with the base sequence GGXGG, which is a homopolymer or a flanked homopolymer (e.g., based on the value of X). In an embodiment, the loss for the middle X (e.g., which is flanked by G's on both sides) for this specific base sequence is penalized differently from penalization of loss for other bases of the sequences, as well as for other general base sequences. For example, the loss for the middle X for this specific base sequence is amplified, by a corresponding amplification of the corresponding penalty of W2 that is greater than 1 (i.e., W2>1).

When the loss penalization module 2106 detects a specific base sequence in the ground truth data, the loss penalization module 2106 applies a specialized amplified weight or penalty to one or more bases of such a specific base sequence. Accordingly, for example, the penalty W2 of the penalization matrix 2208c of FIG. 22C is different (e.g., amplified or higher) from various other penalties of the various penalization matrices 2208. For example, W2 of FIG. 22C is different (e.g., amplified or higher) than w1 of FIGS. 22A, 22B, 22D, and/or 22E.

Note that the loss penalization is performed during a training phase of a neural network based base caller. During the training phase, the ground truth base sequence is known a-priori, e.g., prior to the multiplication discussed with respect to FIGS. 22A, 22B, 22C, 22D and 22E. Thus, it is known in advance to the neural network model as to whether a specific base sequence is to be processed. Accordingly, the penalty W2 corresponding to the middle base of the specific base sequence can be made high in FIG. 22C (e.g., even before performing the operations at FIGS. 22D and 22E and processing the last two bases on the specific base sequence), as discussed herein.

In an example, a memory stores the loss penalization matrices 2208a, 2208b, . . . , 2208e. In case the neural network model anticipates the specific base sequence, the penalty W2 corresponding to the middle base of the specific base sequence is altered (e.g., made high), as discussed with respect to FIG. 22C.

Penalizing the middle base of the specific base sequence GGXGG relatively more than the other bases (e.g., by making W2 relatively higher) amplifies the loss associated with the middle base of the specific base sequence GGXGG. For example, the gradient generated from the penalized loss function 2111 includes the amplified loss for the middle base of the specific base sequence GGXGG. This changes the step size of the gradient descent for this specific base call, which facilitates the neural network model to recognize such specific base sequences and adapt special weights for such specific base sequences.

FIG. 22F illustrates application of a specialized weight to loss associated with a middle base of a specific base sequence. The specific base sequence here is GGXGG, where “X” can be any of A, C, T, or G. However, the teachings of this disclosure are not limited to any specific “specific base sequence,” and can be applied to any homopolymers, flanked homopolymers, or any other specific base sequence discussed herein with respect to FIGS. 19, 20A, and 20B. As seen, a regular penalty w1 (which can be pre-specified and selected in accordance with any appropriate weight selection scheme, and for example, w1=1) is applied to losses associated with all the bases, except for the loss associated with the middle base of the specific base sequence. For the middle base of the specific base sequence, a penalty of W2 is applied to the corresponding loss, where W2 is different from (e.g., higher than) the regular weights.

Loss Penalization Results

FIG. 22G illustrates two graphs 2280 and 2284 comparing the performance of a base calling system that does not penalize loss, versus a base calling system penalizes loss for a specific base sequence. The specific base sequence used in these graphs is GGGGG. The X axis in each of these plots is the predicted quality score 1432, and the Y axis in each of these plots is the true quality score 1440.

Graph 2280 is for a base calling system that does not specifically penalize loss for the specific base sequence GGGGG. As seen, the base calling of the specific sequence in the graph 2280 has an error of 6.4979%.

Graph 2284 is for a base calling system that assigns a penalty of 20 for the middle base of the specific base sequence GGGGG. As seen, the base calling of the specific sequence in the graph 2284 has an error of 1.9941%.

Thus, a penalty of 20 in the graph 2284 drastically improves the error from 6.4979 to 1.9941. Thus, loss penalization, as discussed herein, improves the quality scores, e.g., by better aligning the quality scores to the true (or empirically determined) quality scores.

### Example Applications of Quality Score Calibration

This disclosure discusses various approaches for calibration of quality scores, e.g., such that the calibrated quality scores are better aligned to the true quality scores. The quality scores may or may not change the underlying base calls.

For example, assume that without calibration, the quality scores associated with a base are as follows: Q(A)=70, and each of Q(C), Q(T), and Q(G) is less than one. The base being called without calibration is A. Assume that when using one or more of the calibration approaches discussed herein (e.g., input normalization, score remapping, and/or loss penalization), the calibrated quality scores are as follows: Q(A)=10, and each of Q(C), Q(T), and Q(G) is less than two. The base being called with calibration is still A. Thus, the calibration does not change the underlying base call. However, although the calibration may or may not change the underlying base call, providing an accurate quality score and underlying accurate confidence level is important in many practical applications. For example, often times, the quality scores are used to make critical health care decisions. For example, in a healthcare setting, confidence scores associated with detecting bases of a human tissue sample may affect an approach to treat a health condition. For example, high quality scores (i.e., high confidence level) in multiple bases of the sample can indicate a high probability of cancer, whereas low quality scores (i.e., low confidence level) in multiple bases of the sample can indicate a questionable probability of cancer—treatment decisions, thus, can change based on the quality score levels. Accordingly, calibrating the quality scores and reporting calibrated quality scores helps in deciding various downstream tasks, which may possibly include healthcare decisions that are associated with levels of quality scores.

Combined Base Calling System Implementing Normalization, Remapping and Quantization, and Loss Penalization

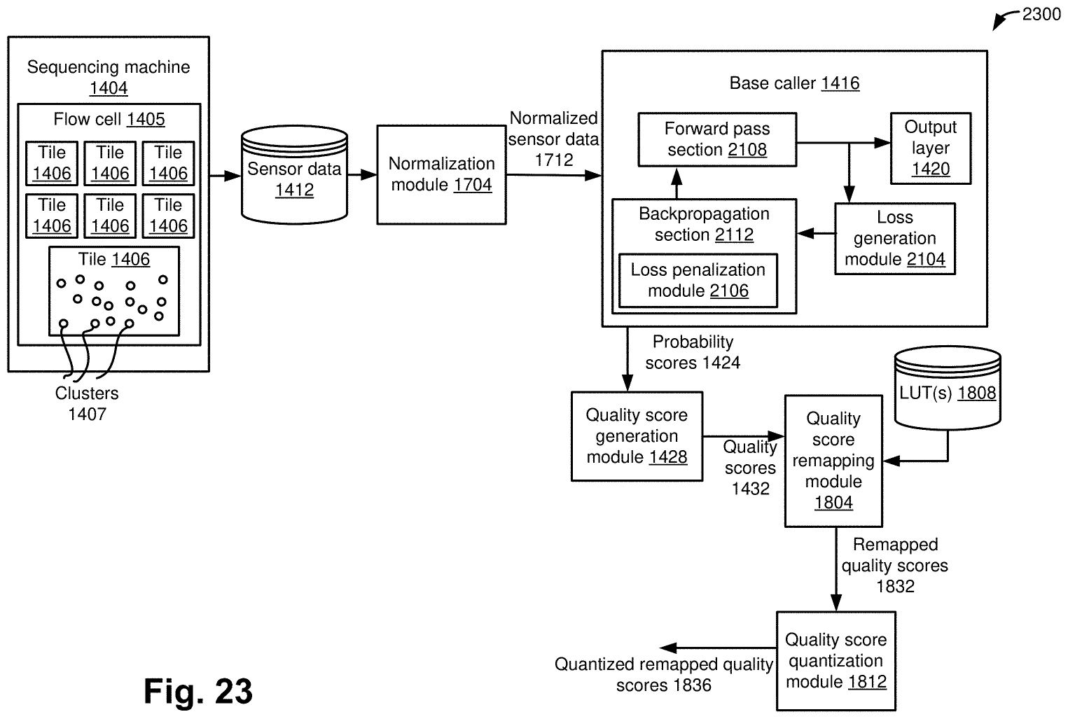

FIG. 23 illustrates a base calling system 2300 that includes (i) the normalization module 1704 of the base calling system 1700 of FIG. 17A, (ii) the quality score remapping module 1804 and the quality score quantization module 1812 of the base calling system 1800 of FIG. 18A, and (iii) the loss penalization module 2106 of the base calling system 2100 of FIG. 21. Thus, the base calling system 2300 can perform one or more of input normalization, quality score remapping and quantization, and/or loss penalization, as discussed throughout this disclosure.

Base Calling Architecture

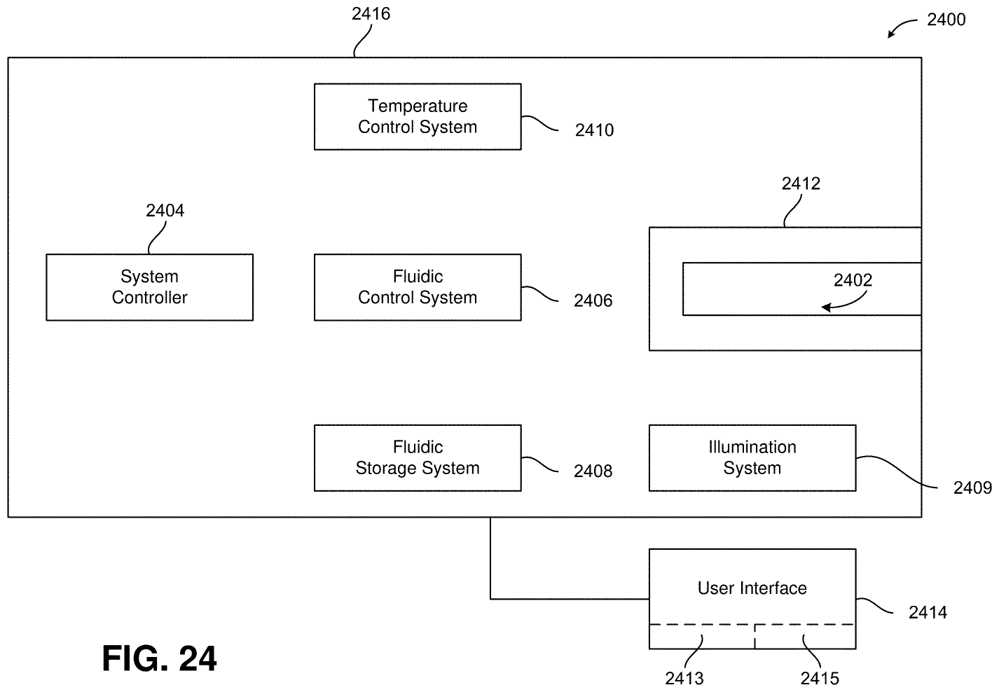

FIG. 24 is a block diagram of a base calling system 2400 in accordance with one implementation. The base calling system 2400 may operate to obtain any information or data that relates to at least one of a biological or chemical substance. In some implementations, the base calling system 2400 is a workstation that may be similar to a bench-top device or desktop computer. For example, a majority (or all) of the systems and components for conducting the desired reactions can be within a common housing 2416.

In particular implementations, the base calling system 2400 is a nucleic acid sequencing system (or sequencer) configured for various applications, including but not limited to de novo sequencing, resequencing of whole genomes or target genomic regions, and metagenomics. The sequencer may also be used for DNA or RNA analysis. In some implementations, the base calling system 2400 may also be configured to generate reaction sites in a biosensor. For example, the base calling system 2400 may be configured to receive a sample and generate surface attached clusters of clonally amplified nucleic acids derived from the sample. Each cluster may constitute or be part of a reaction site in the biosensor.

The exemplary base calling system 2400 may include a system receptacle or interface 2412 that is configured to interact with a biosensor 2402 to perform desired reactions within the biosensor 2402. In the following description with respect to FIG. 24, the biosensor 2402 is loaded into the system receptacle 2412. However, it is understood that a cartridge that includes the biosensor 2402 may be inserted into the system receptacle 2412 and in some states the cartridge can be removed temporarily or permanently. As described above, the cartridge may include, among other things, fluidic control and fluidic storage components.

In particular implementations, the base calling system 2400 is configured to perform a large number of parallel reactions within the biosensor 2402. The biosensor 2402 includes one or more reaction sites where desired reactions can occur. The reaction sites may be, for example, immobilized to a solid surface of the biosensor or immobilized to beads (or other movable substrates) that are located within corresponding reaction chambers of the biosensor. The reaction sites can include, for example, clusters of clonally amplified nucleic acids. The biosensor 2402 may include a solid-state imaging device (e.g., CCD or CMOS imager) and a flow cell mounted thereto. The flow cell may include one or more flow channels that receive a solution from the base calling system 2400 and direct the solution toward the reaction sites. Optionally, the biosensor 2402 can be configured to engage a thermal element for transferring thermal energy into or out of the flow channel.

The base calling system 2400 may include various components, assemblies, and systems (or sub-systems) that interact with each other to perform a predetermined method or assay protocol for biological or chemical analysis. For example, the base calling system 2400 includes a system controller 2404 that may communicate with the various components, assemblies, and sub-systems of the base calling system 2400 and also the biosensor 2402. For example, in addition to the system receptacle 2412, the base calling system 2400 may also include a fluidic control system 2406 to control the flow of fluid throughout a fluid network of the base calling system 2400 and the biosensor 2402; a fluidic storage system 2408 that is configured to hold all fluids (e.g., gas or liquids) that may be used by the bioassay system; a temperature control system 2410 that may regulate the temperature of the fluid in the fluid network, the fluidic storage system 2408, and/or the biosensor 2402; and an illumination system 2409 that is configured to illuminate the biosensor 2402. As described above, if a cartridge having the biosensor 2402 is loaded into the system receptacle 2412, the cartridge may also include fluidic control and fluidic storage components.

Also shown, the base calling system 2400 may include a user interface 2414 that interacts with the user. For example, the user interface 2414 may include a display 2413 to display or request information from a user and a user input device 2415 to receive user inputs. In some implementations, the display 2413 and the user input device 2415 are the same device. For example, the user interface 2414 may include a touch-sensitive display configured to detect the presence of an individual's touch and also identify a location of the touch on the display. However, other user input devices 2415 may be used, such as a mouse, touchpad, keyboard, keypad, handheld scanner, voice-recognition system, motion-recognition system, and the like. As will be discussed in greater detail below, the base calling system 2400 may communicate with various components, including the biosensor 2402 (e.g., in the form of a cartridge), to perform the desired reactions. The base calling system 2400 may also be configured to analyze data obtained from the biosensor to provide a user with desired information.

The system controller 2404 may include any processor-based or microprocessor-based system, including systems using microcontrollers, Reduced Instruction Set Computers (RISC), Application Specific Integrated Circuits (ASICs), Field Programmable Gate Array (FPGAs), logic circuits, and any other circuit or processor capable of executing functions described herein. The above examples are exemplary only, and are thus not intended to limit in any way the definition and/or meaning of the term system controller. In the exemplary implementation, the system controller 2404 executes a set of instructions that are stored in one or more storage elements, memories, or modules in order to at least one of obtain and analyze detection data. Detection data can include a plurality of sequences of pixel signals, such that a sequence of pixel signals from each of the millions of sensors (or pixels) can be detected over many base calling cycles. Storage elements may be in the form of information sources or physical memory elements within the base calling system 2400.

The set of instructions may include various commands that instruct the base calling system 2400 or biosensor 2402 to perform specific operations such as the methods and processes of the various implementations described herein. The set of instructions may be in the form of a software program, which may form part of a tangible, non-transitory computer readable medium or media. As used herein, the terms “software” and “firmware” are interchangeable and include any computer program stored in memory for execution by a computer, including RAM memory, ROM memory, EPROM memory, EEPROM memory, and non-volatile RAM (NVRAM) memory.

The above memory types are exemplary only, and are thus not limiting as to the types of memory usable for storage of a computer program.

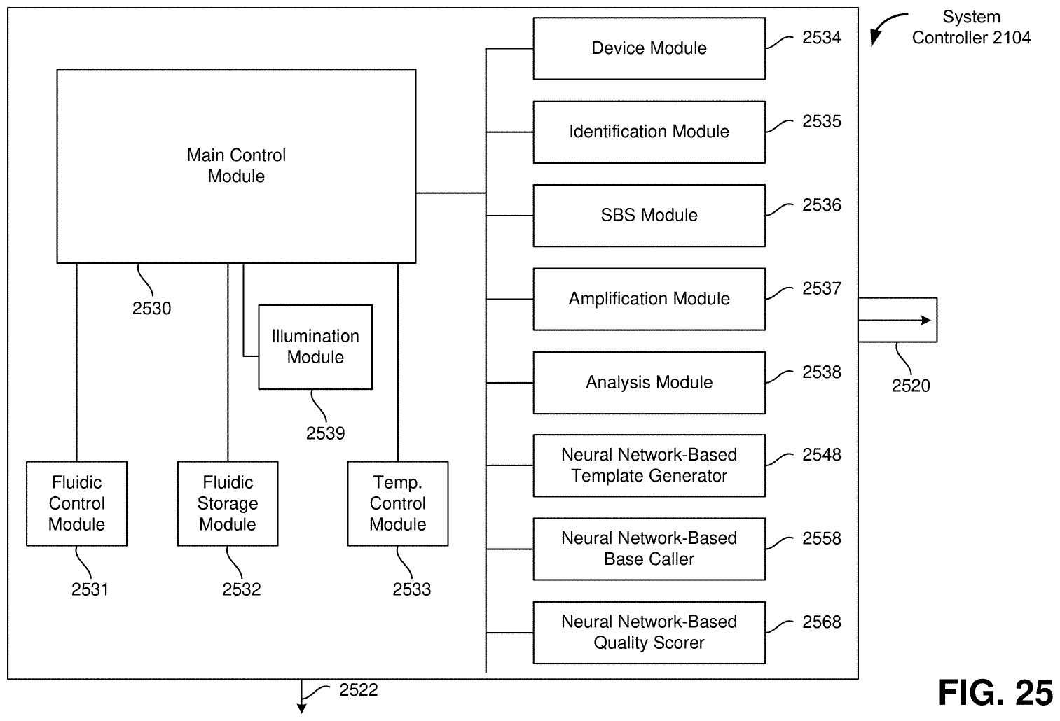

The software may be in various forms such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the base calling system 2400, processed in response to user inputs, or processed in response to a request made by another processing machine (e.g., a remote request through a communication link). In the illustrated implementation, the system controller 2404 includes an analysis module 2538 (illustrated in FIG. 25). In other implementations, system controller 2404 does not include the analysis module 2538 and instead has access to the analysis module 2538 (e.g., the analysis module 2538 may be separately hosted on cloud).

The system controller 2404 may be connected to the biosensor 2402 and the other components of the base calling system 2400 via communication links. The system controller 2404 may also be communicatively connected to off-site systems or servers. The communication links may be hardwired, corded, or wireless. The system controller 2404 may receive user inputs or commands, from the user interface 2414 and the user input device 2415.

The fluidic control system 2406 includes a fluid network and is configured to direct and regulate the flow of one or more fluids through the fluid network. The fluid network may be in fluid communication with the biosensor 2402 and the fluidic storage system 2408. For example, select fluids may be drawn from the fluidic storage system 2408 and directed to the biosensor 2402 in a controlled manner, or the fluids may be drawn from the biosensor 2402 and directed toward, for example, a waste reservoir in the fluidic storage system 2408. Although not shown, the fluidic control system 2406 may include flow sensors that detect a flow rate or pressure of the fluids within the fluid network. The sensors may communicate with the system controller 2404.

The temperature control system 2410 is configured to regulate the temperature of fluids at different regions of the fluid network, the fluidic storage system 2408, and/or the biosensor 2402. For example, the temperature control system 2410 may include a thermocycler that interfaces with the biosensor 2402 and controls the temperature of the fluid that flows along the reaction sites in the biosensor 2402. The temperature control system 2410 may also regulate the temperature of solid elements or components of the base calling system 2400 or the biosensor 2402. Although not shown, the temperature control system 2410 may include sensors to detect the temperature of the fluid or other components. The sensors may communicate with the system controller 2404.

The fluidic storage system 2408 is in fluid communication with the biosensor 2402 and may store various reaction components or reactants that are used to conduct the desired reactions therein. The fluidic storage system 2408 may also store fluids for washing or cleaning the fluid network and biosensor 2402 and for diluting the reactants. For example, the fluid storage system 2408 may include various reservoirs to store samples, reagents, enzymes, other biomolecules, buffer solutions, aqueous, and non-polar solutions, and the like. Furthermore, the fluidic storage system 2408 may also include waste reservoirs for receiving waste products from the biosensor 2402. In implementations that include a cartridge, the cartridge may include one or more of a fluid storage system, fluidic control system or temperature control system. Accordingly, one or more of the components set forth herein as relating to those systems can be contained within a cartridge housing. For example, a cartridge can have various reservoirs to store samples, reagents, enzymes, other biomolecules, buffer solutions, aqueous, and non-polar solutions, waste, and the like. As such, one or more of a fluid storage system, fluidic control system or temperature control system can be removably engaged with a bioassay system via a cartridge or other biosensor.

The illumination system 2409 may include a light source (e.g., one or more LEDs) and a plurality of optical components to illuminate the biosensor. Examples of light sources may include lasers, arc lamps, LEDs, or laser diodes. The optical components may be, for example, reflectors, dichroics, beam splitters, collimators, lenses, filters, wedges, prisms, mirrors, detectors, and the like. In implementations that use an illumination system, the illumination system 2409 may be configured to direct an excitation light to reaction sites. As one example, fluorophores may be excited by green wavelengths of light, as such the wavelength of the excitation light may be approximately 532 nm. In one implementation, the illumination system 2409 is configured to produce illumination that is parallel to a surface normal of a surface of the biosensor 2402. In another implementation, the illumination system 2409 is configured to produce illumination that is off-angle relative to the surface normal of the surface of the biosensor 2402. In yet another implementation, the illumination system 2409 is configured to produce illumination that has plural angles, including some parallel illumination and some off-angle illumination.

The system receptacle or interface 2412 is configured to engage the biosensor 2402 in at least one of a mechanical, electrical, and fluidic manner. The system receptacle 2412 may hold the biosensor 2402 in a desired orientation to facilitate the flow of fluid through the biosensor 2402. The system receptacle 2412 may also include electrical contacts that are configured to engage the biosensor 2402 so that the base calling system 2400 may communicate with the biosensor 2402 and/or provide power to the biosensor 2402. Furthermore, the system receptacle 2412 may include fluidic ports (e.g., nozzles) that are configured to engage the biosensor 2402. In some implementations, the biosensor 2402 is removably coupled to the system receptacle 2412 in a mechanical manner, in an electrical manner, and also in a fluidic manner.

In addition, the base calling system 2400 may communicate remotely with other systems or networks or with other bioassay systems 2400. Detection data obtained by the bioassay system(s) 2400 may be stored in a remote database.

FIG. 25 is a block diagram of the system controller 2404 that can be used in the system of FIG. 24. In one implementation, the system controller 2404 includes one or more processors or modules that can communicate with one another. Each of the processors or modules may include an algorithm (e.g., instructions stored on a tangible and/or non-transitory computer readable storage medium) or sub-algorithms to perform particular processes. The system controller 2404 is illustrated conceptually as a collection of modules, but may be implemented utilizing any combination of dedicated hardware boards, DSPs, processors, etc. Alternatively, the system controller 2404 may be implemented utilizing an off-the-shelf PC with a single processor or multiple processors, with the functional operations distributed between the processors. As a further option, the modules described below may be implemented utilizing a hybrid configuration in which certain modular functions are performed utilizing dedicated hardware, while the remaining modular functions are performed utilizing an off-the-shelf PC and the like. The modules also may be implemented as software modules within a processing unit.

During operation, a communication port 2520 may transmit information (e.g., commands) to or receive information (e.g., data) from the biosensor 2402 (FIG. 24) and/or the sub-systems 2406, 2408, 2410 (FIG. 24). In implementations, the communication port 2520 may output a plurality of sequences of pixel signals. A communication port 2520 may receive user input from the user interface 2414 (FIG. 24) and transmit data or information to the user interface 2414. Data from the biosensor 2402 or sub-systems 2406, 2408, 2410 may be processed by the system controller 2404 in real-time during a bioassay session. Additionally, or alternatively, data may be stored temporarily in a system memory during a bioassay session and processed in slower than real-time or off-line operation.

As shown in FIG. 25, the system controller 2404 may include a plurality of modules 2531-2539 that communicate with a main control module 2530. The main control module 2530 may communicate with the user interface 2414 (FIG. 24). Although the modules 2531-2539 are shown as communicating directly with the main control module 2530, the modules 2531-2539 may also communicate directly with each other, the user interface 2414, and the biosensor 2402. Also, the modules 2531-2539 may communicate with the main control module 2530 through the other modules.

The plurality of modules 2531-2539 include system modules 2531-2533, 2539 that communicate with the sub-systems 2406, 2408, 2410, and 2409, respectively. The fluidic control module 2531 may communicate with the fluidic control system 2406 to control the valves and flow sensors of the fluid network for controlling the flow of one or more fluids through the fluid network. The fluidic storage module 2532 may notify the user when fluids are low or when the waste reservoir is at or near capacity. The fluidic storage module 2532 may also communicate with the temperature control module 2533 so that the fluids may be stored at a desired temperature. The illumination module 2539 may communicate with the illumination system 2409 to illuminate the reaction sites at designated times during a protocol, such as after the desired reactions (e.g., binding events) have occurred. In some implementations, the illumination module 2539 may communicate with the illumination system 2409 to illuminate the reaction sites at designated angles.

The plurality of modules 2531-2539 may also include a device module 2534 that communicates with the biosensor 2402 and an identification module 2535 that determines identification information relating to the biosensor 2402. The device module 2534 may, for example, communicate with the system receptacle 2412 to confirm that the biosensor has established an electrical and fluidic connection with the base calling system 2400. The identification module 2535 may receive signals that identify the biosensor 2402. The identification module 2535 may use the identity of the biosensor 2402 to provide other information to the user. For example, the identification module 2535 may determine and then display a lot number, a date of manufacture, or a protocol that is recommended to be run with the biosensor 2402.

The plurality of modules 2531-2539 also includes an analysis module 2538 (also called signal processing module or signal processor) that receives and analyzes the signal data (e.g., image data) from the biosensor 2402. Analysis module 2538 includes memory (e.g., RAM or Flash) to store detection data. Detection data can include a plurality of sequences of pixel signals, such that a sequence of pixel signals from each of the millions of sensors (or pixels) can be detected over many base calling cycles. The signal data may be stored for subsequent analysis or may be transmitted to the user interface 2414 to display desired information to the user. In some implementations, the signal data may be processed by the solid-state imager (e.g., CMOS image sensor) before the analysis module 2538 receives the signal data.

The analysis module 2538 is configured to obtain image data from the light detectors at each of a plurality of sequencing cycles. The image data is derived from the emission signals detected by the light detectors and process the image data for each of the plurality of sequencing cycles through a neural network (e.g., a neural network-based template generator 2548, a neural network-based base caller 2558 (e.g., see FIGS. 7, 9, and 10), and/or a neural network-based quality scorer 2568) and produce a base call for at least some of the analytes at each of the plurality of sequencing cycle.

Protocol modules 2536 and 2537 communicate with the main control module 2530 to control the operation of the sub-systems 2406, 2408, and 2410 when conducting predetermined assay protocols. The protocol modules 2536 and 2537 may include sets of instructions for instructing the base calling system 2400 to perform specific operations pursuant to predetermined protocols. As shown, the protocol module may be a sequencing-by-synthesis (SBS) module 2536 that is configured to issue various commands for performing sequencing-by-synthesis processes. In SBS, extension of a nucleic acid primer along a nucleic acid template is monitored to determine the sequence of nucleotides in the template. The underlying chemical process can be polymerization (e.g., as catalyzed by a polymerase enzyme) or ligation (e.g., catalyzed by a ligase enzyme). In a particular polymerase-based SBS implementation, fluorescently labeled nucleotides are added to a primer (thereby extending the primer) in a template dependent fashion such that detection of the order and type of nucleotides added to the primer can be used to determine the sequence of the template. For example, to initiate a first SBS cycle, commands can be given to deliver one or more labeled nucleotides, DNA polymerase, etc., into/through a flow cell that houses an array of nucleic acid templates. The nucleic acid templates may be located at corresponding reaction sites. Those reaction sites where primer extension causes a labeled nucleotide to be incorporated can be detected through an imaging event. During an imaging event, the illumination system 2409 may provide an excitation light to the reaction sites. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for implementations that use reversible termination a command can be given to deliver a deblocking reagent to the flow cell (before or after detection occurs). One or more commands can be given to effect wash(es) between the various delivery steps. The cycle can then be repeated n times to extend the primer by n nucleotides, thereby detecting a sequence of length n. Exemplary sequencing techniques are described, for example, in Bentley et al., Nature 456:53-59 (2008); WO 04/018497; U.S. Pat. No. 7,057,026; WO 91/06678; WO 07/123744; U.S. Pat. Nos. 7,329,492; 7,211,414; 7,315,019; and 7,405,281, each of which is incorporated herein by reference.

For the nucleotide delivery step of an SBS cycle, either a single type of nucleotide can be delivered at a time, or multiple different nucleotide types (e.g., A, C, T and G together) can be delivered. For a nucleotide delivery configuration where only a single type of nucleotide is present at a time, the different nucleotides need not have distinct labels since they can be distinguished based on temporal separation inherent in the individualized delivery. Accordingly, a sequencing method or apparatus can use single color detection. For example, an excitation source need only provide excitation at a single wavelength or in a single range of wavelengths. For a nucleotide delivery configuration where delivery results in multiple different nucleotides being present in the flow cell at one time, sites that incorporate different nucleotide types can be distinguished based on different fluorescent labels that are attached to respective nucleotide types in the mixture. For example, four different nucleotides can be used, each having one of four different fluorophores. In one implementation, the four different fluorophores can be distinguished using excitation in four different regions of the spectrum. For example, four different excitation radiation sources can be used. Alternatively, fewer than four different excitation sources can be used, but optical filtration of the excitation radiation from a single source can be used to produce different ranges of excitation radiation at the flow cell.

In some implementations, fewer than four different colors can be detected in a mixture having four different nucleotides. For example, pairs of nucleotides can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. Exemplary apparatus and methods for distinguishing four different nucleotides using detection of fewer than four colors are described for example in US Pat. App. Ser. Nos. 61/538,294 and 61/619,878, which are incorporated herein by reference in their entireties. U.S. application Ser. No. 13/624,200, which was filed on Sep. 21, 2012, is also incorporated by reference in its entirety.

The plurality of protocol modules may also include a sample-preparation (or generation) module 2537 that is configured to issue commands to the fluidic control system 2406 and the temperature control system 2410 for amplifying a product within the biosensor 2402. For example, the biosensor 2402 may be engaged to the base calling system 2400. The amplification module 2537 may issue instructions to the fluidic control system 2406 to deliver necessary amplification components to reaction chambers within the biosensor 2402. In other implementations, the reaction sites may already contain some components for amplification, such as the template DNA and/or primers. After delivering the amplification components to the reaction chambers, the amplification module 2537 may instruct the temperature control system 2410 to cycle through different temperature stages according to known amplification protocols. In some implementations, the amplification and/or nucleotide incorporation is performed isothermally.

The SBS module 2536 may issue commands to perform bridge PCR where clusters of clonal amplicons are formed on localized areas within a channel of a flow cell. After generating the amplicons through bridge PCR, the amplicons may be “linearized” to make single stranded template DNA, or sstDNA, and a sequencing primer may be hybridized to a universal sequence that flanks a region of interest. For example, a reversible terminator-based sequencing by synthesis method can be used as set forth above or as follows.

Each base calling or sequencing cycle can extend an sstDNA by a single base which can be accomplished for example by using a modified DNA polymerase and a mixture of four types of nucleotides. The different types of nucleotides can have unique fluorescent labels, and each nucleotide can further have a reversible terminator that allows only a single-base incorporation to occur in each cycle. After a single base is added to the sstDNA, excitation light may be incident upon the reaction sites and fluorescent emissions may be detected. After detection, the fluorescent label and the terminator may be chemically cleaved from the sstDNA. Another similar base calling or sequencing cycle may follow. In such a sequencing protocol, the SBS module 2536 may instruct the fluidic control system 2406 to direct a flow of reagent and enzyme solutions through the biosensor 2402. Exemplary reversible terminator-based SBS methods which can be utilized with the apparatus and methods set forth herein are described in US Patent Application Publication No. 2007/0166705 A1, US Patent Application Publication No. 2006/0188901 A1, U.S. Pat. No. 7,057,026, US Patent Application Publication No. 2006/0240439 A1, US Patent Application Publication No. 2006/02814714709 A1, PCT Publication No. WO 05/065814, PCT Publication No. WO 06/064199, each of which is incorporated herein by reference in its entirety. Exemplary reagents for reversible terminator-based SBS are described in U.S. Pat. Nos. 7,541,444; 7,057,026; 7,427,673; 7,566,537; and 7,592,435, each of which is incorporated herein by reference in its entirety.

In some implementations, the amplification and SBS modules may operate in a single assay protocol where, for example, template nucleic acid is amplified and subsequently sequenced within the same cartridge.

The base calling system 2400 may also allow the user to reconfigure an assay protocol. For example, the base calling system 2400 may offer options to the user through the user interface 2414 for modifying the determined protocol. For example, if it is determined that the biosensor 2402 is to be used for amplification, the base calling system 2400 may request a temperature for the annealing cycle. Furthermore, the base calling system 2400 may issue warnings to a user if a user has provided user inputs that are generally not acceptable for the selected assay protocol.

In implementations, the biosensor 2402 includes millions of sensors (or pixels), each of which generates a plurality of sequences of pixel signals over successive base calling cycles. The analysis module 2538 detects the plurality of sequences of pixel signals and attributes them to corresponding sensors (or pixels) in accordance to the row-wise and/or column-wise location of the sensors on an array of sensors.

Each sensor in the array of sensors can produce sensor data for a tile of the flow cell, where a tile in an area on the flow cell at which clusters of genetic material are disposed during the based calling operation. The sensor data can comprise image data in an array of pixels. For a given cycle, the sensor data can include more than one image, producing multiple features per pixel as the tile data.

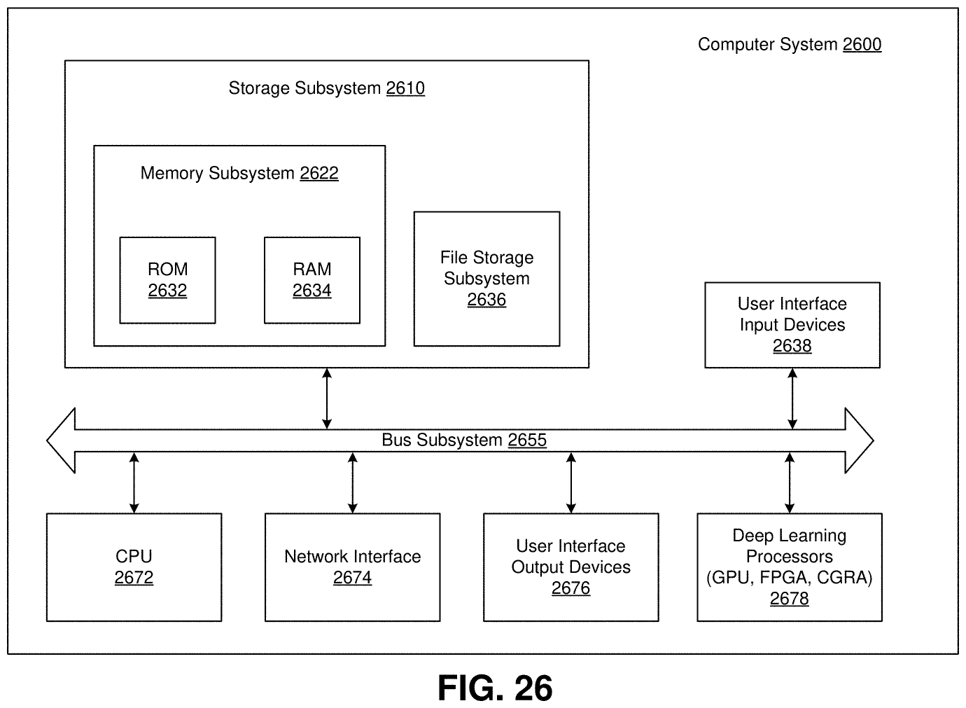

FIG. 26 is a simplified block diagram of a computer 2600 system that can be used to implement the technology disclosed. Computer system 2600 includes at least one central processing unit (CPU) 2672 that communicates with a number of peripheral devices via bus subsystem 2655. These peripheral devices can include a storage subsystem 2610 including, for example, memory devices and a file storage subsystem 2636, user interface input devices 2638, user interface output devices 2676, and a network interface subsystem 2674. The input and output devices allow user interaction with computer system 2600. Network interface subsystem 2674 provides an interface to outside networks, including an interface to corresponding interface devices in other computer systems.

User interface input devices 2638 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 2600.

User interface output devices 2676 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 2600 to the user or to another machine or computer system.

Storage subsystem 2610 stores programming and data constructs that provide the functionality of some or all of the modules and methods described herein. These software modules are generally executed by deep learning processors 2678.

In one implementation, the neural networks are implemented using deep learning processors 2678 can be configurable and reconfigurable processors, field-programmable gate arrays (FPGAs), application-specific integrated circuits (ASICs), and/or coarse-grained reconfigurable architectures (CGRAs) and graphics processing units (GPUs) other configured devices. Deep learning processors 2678 can be hosted by a deep learning cloud platform such as Google Cloud Platform™, Xilinx™, and Cirrascale™. Examples of deep learning processors 2678 include Google's Tensor Processing Unit (TPU)™, rackmount solutions like GX4 Rackmount Series™, GX149 Rackmount Series™, NVIDIA DGX-1™, Microsoft' Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamiclQ™, IBM TrueNorth™, and others.

Memory subsystem 2622 used in the storage subsystem 2610 can include a number of memories including a main random access memory (RAM) 2634 for storage of instructions and data during program execution and a read only memory (ROM) 2632 in which fixed instructions are stored. A file storage subsystem 2636 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 2636 in the storage subsystem 2610, or in other machines accessible by the processor.

Bus subsystem 2655 provides a mechanism for letting the various components and subsystems of computer system 2600 communicate with each other as intended. Although bus subsystem 2655 is shown schematically as a single bus, alternative implementations of the bus subsystem can use multiple busses.

Computer system 2600 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 2600 depicted in FIG. 26 is intended only as a specific example for purposes of illustrating the preferred implementations of the present invention. Many other configurations of computer system 2600 are possible having more or less components than the computer system depicted in FIG. 26.

### CLAUSES

The following clauses are part of this disclosure:

### Clause Set 1 (Input Normalization)

1. A computer-implemented method of generating base calls by a base caller, including:
   receiving a plurality of sensor data from a flow cell, wherein the plurality of sensor data is within a first range;
   identifying a second range, such that at least a threshold percentage of the plurality of sensor data are within the second range;
   mapping at least a subset of the plurality of sensor data, that are within the second range, to a third range, thereby generating a plurality of normalized sensor data; and
   processing the plurality of normalized sensor data in a base caller, to call, for the plurality of normalized sensor data, one or more corresponding bases.
2. The method of clause 1, wherein the second range is fully encompassed within the first range.
3. The method of clause 1, wherein one or more outlier sensor data within the first range are absent from the second range of sensor data.
4. The method of clause 1, wherein identifying the second range comprises:
   identifying, within the first range, a low value, such that a lower threshold percentage of the plurality of sensor data have a value that is lower than the low value; and
   identifying, within the first range, a high value, such that an upper threshold percentage of the plurality of sensor data have a value that is higher than the high value,
   wherein the second range is defined by the low value and the high value.
5. The method of clause 4, wherein at least one of the lower threshold percentage or the upper threshold percentage is 0.5% or less.
6. The method of clause 4, wherein at least one of the lower threshold percentage or the upper threshold percentage is 1.0% or less.
7. The method of clause 4, wherein each of the lower threshold percentage and the upper threshold percentage is 0.5% or less.
8. The method of clause 4, wherein each of the lower threshold percentage and the upper threshold percentage is 1% or less.
9. The method of clause 4, further comprising:
   identifying (i) a first outlier sensor data of the plurality of sensor data that is lower than the low value and (ii) a second outlier sensor data of the plurality of sensor data that is higher than the high value; and
   prior to the mapping, assigning the low value to the first outlier sensor data, and assigning the high value to the second outlier sensor data, such that the first outlier sensor data and the second outlier sensor data are within the second range subsequent to the assignment.
10. The method of clause 4, further comprising:
   identifying (i) a first outlier sensor data of the plurality of sensor data that is lower than the low value and (ii) a second outlier sensor data of the plurality of sensor data that is higher than the high value; and
   excluding the first outlier sensor data and the second outlier sensor data from the subset of the plurality of sensor data during the mapping, for being outside the second range, such that the first outlier sensor data and the second outlier sensor data are not mapped to the third range.
11. The method of clause 1, wherein mapping at least a subset of the plurality of sensor data comprises:
   mapping a first sensor data within the subset from a first value that is within the second range to a second value that is within the third range; and
   mapping a second sensor data within the subset from a third value that is within the second range to a fourth value that is within the third range.
12. The method of clause 1, wherein at least a part of the second range is non-overlapping with the third range.
13. The method of clause 1, wherein individual sensor data of the plurality of sensor data comprises corresponding intensity of a corresponding section of an image generated from the flow cell.
14. The method of clause 1, further comprising:
   processing the plurality of normalized sensor data in a base caller, to assign, for each base call, a first quality score indicating a probability of the called base being an A, a second quality score indicating a probability of the called base being a C, a third quality score indicating a probability of the called base being a T, and a fourth quality score indicating a probability of the called base being a G.
15. The method of clause 14, further comprising:
   assigning a plurality of quality scores that includes the first quality score, the second quality score, the third quality score, and the fourth quality score; and
   remapping each of at least a subset of the plurality of quality scores to a corresponding remapped quality score.
16. The method of clause 15, further comprising:
   quantizing each of a plurality of remapped quality scores to a corresponding one of a plurality of quantized remapped quality score.
17. A non-transitory computer readable storage medium impressed with computer program instructions that, when executed on a processor, implement a method comprising:
   receiving a plurality of intensity values from a flow cell, wherein an individual intensity value depicts a target cluster or an immediate vicinity of the target cluster of the flow cell, the target cluster populated with an unknown analyte;
   identifying a second range that includes at least a threshold percentage of the plurality of intensity values;
   mapping the threshold percentage of the plurality of intensity values to a third range that is different from the second range; and
   subsequent to the mapping, processing the threshold percentage of the plurality of intensity values, to generate likelihoods of the unknown analyte being an A, C, T, or G.
18. The non-transitory computer readable storage medium of clause 17, wherein the second range is fully encompassed within the first range.
19. The non-transitory computer readable storage medium of clause 17, wherein one or more outlier intensity values within the first range are absent from the threshold percentage of the plurality of intensity value.
20. The non-transitory computer readable storage medium of clause 17, wherein identifying the second range comprises:
   identifying, within the first range, a low value, such that a lower threshold percentage of the plurality of intensity values have a value that is lower than the low value; and
   identifying, within the first range, a high value, such that an upper threshold percentage of the plurality of intensity values have a value that is higher than the high value, wherein the threshold percentage is a sum of the lower threshold percentage and the higher threshold percentage,
   wherein the second range is defined by the low value and the high value.
21. The non-transitory computer readable storage medium of clause 20, wherein at least one of the lower threshold percentage or the upper threshold percentage is 0.5% or less.
22. The non-transitory computer readable storage medium of clause 20, wherein each of the lower threshold percentage and the upper threshold percentage is 1.0% or less.
23. The non-transitory computer readable storage medium of clause 20, further comprising:
identifying (i) a first outlier intensity value of the plurality of intensity values that is lower than the low value and (ii) a second outlier intensity value of the plurality of intensity values that is higher than the high value; and
prior to the mapping, assigning the low value to the first outlier intensity value, and assigning the high value to the second outlier intensity value, such that the first outlier intensity value and the second outlier intensity value are within the second range subsequent to the assignment.
24. The non-transitory computer readable storage medium of clause 20, further comprising:
   identifying (i) a first outlier intensity value of the plurality of intensity values that is lower than the low value and (ii) a second outlier intensity value of the plurality of intensity values that is higher than the high value; and
   excluding the first outlier intensity value and the second outlier intensity value from the subset of the plurality of intensity values during the mapping, for being outside the second range, such that the first outlier intensity value and the second outlier intensity value are not mapped to the third range.
25. The non-transitory computer readable storage medium of clause 17, wherein the mapping comprises:
   mapping a first intensity value from a first value that is within the second range to a second value that is within the third range; and
   mapping a second intensity value from a third value that is within the second range to a fourth value that is within the third range.
26. The non-transitory computer readable storage medium of clause 17, wherein at least a part of the second range is non-overlapping with the third range.
27. A system for base calling, comprising:
   memory storing images that depict original intensity emissions of a set of analytes, the original intensity emissions generated by analytes in the set of analytes during sequencing cycles of a sequencing run;
   a normalization module configured to receive the original intensity emissions and remap the original intensity emissions to generate remapped intensity emissions, such that a remapped intensity emission has a different intensity value relative to the original intensity emission; and a base caller configured to process the remapped intensity emissions, to generate base calls for the set of analytes.

### Clause Set 2 (Quality Score Remapping)

1. A computer-implemented method of calibrating quality scores generated by a base caller, comprising:
   processing sensor data in a base caller, to generate a plurality of probability scores, wherein each of the plurality of probability scores identifies a corresponding likelihood of a base being a corresponding one of A, C, T, or G;
   transforming each probability score to a corresponding quality score, thereby generating a plurality of quality scores corresponding to the plurality of probability scores, wherein each of the plurality of quality scores indicates, in a logarithmic scale, a corresponding likelihood of a base being a corresponding one of A, C, T, or G; and
   remapping one or more of the plurality of quality scores, to generate a corresponding plurality of remapped quality scores.
2. The method of clause 1, wherein:
   a first quality score of the plurality of quality scores is remapped to a first remapped quality score of the plurality of remapped quality scores;
   the first quality score indicates a first likelihood of a corresponding first base being an X, where X is one of A, C, T, and G;
   the first remapped quality score indicates a first remapped likelihood of the corresponding first base being the X; and
   the first remapped likelihood is more aligned to an empirically determined likelihood of the corresponding first base being the X, compared to an alignment of the first remapped likelihood to the empirically determined likelihood.
3. The method of clause 2, wherein the first quality score indicates the first likelihood in the logarithmic scale, and the first remapped quality score indicates the first remapped likelihood in the logarithmic scale.
4. The method of clause 2, wherein:
   a difference between the first remapped likelihood and the empirically determined likelihood is less than a difference between the first likelihood and the empirically determined likelihood.
5. The method of clause 1, wherein the remapping comprises:
   identifying, from a lookup table (LUT), that a first quality score of the plurality of quality scores is to remap to a first remapped quality score; and
   assigning the first remapped quality score to the first quality score, thereby remapping the first quality score to the first remapped quality score of the plurality of remapped quality scores.
6. The method of clause 1, wherein the remapping comprises:
   using a lookup table (LUT) to remap one or more of the plurality of quality scores, to generate the corresponding plurality of remapped quality scores.
7. The method of clause 6, wherein the LUT identifies, for one or more quality scores, corresponding one or more remapped quality scores.
8. The method of clause 1, wherein transforming each probability score to a corresponding quality score comprises:
   transforming a probability score P to a corresponding quality score Q by using the equation: Q=−10×log₁₀(1−P).
9. The method of clause 1, further comprising:
   reporting the plurality of remapped quality scores, which provides a more accurate indication of confidence levels in the base calls relative to the confidence levels associated with the plurality of quality scores.
10. The method of clause 1, further comprising:
   including each of the plurality of remapped quality scores in a corresponding one of a plurality of groups, such that a first group of the plurality of groups includes a first subset of the plurality of remapped quality scores, and a second group of the plurality of groups includes a second subset of the plurality of remapped quality scores;
   assigning, to each of the first subset of the plurality of remapped quality scores included in the first group, a first quantized quality score; and
   assigning, to each of the second subset of the plurality of remapped quality scores included in the second group, a second quantized quality score.
11. The method of clause 10, wherein including each of the plurality of remapped quality scores in a corresponding one of the plurality of groups comprises:
   assigning, to each group of the plurality of groups, a corresponding range of remapped quality scores;
   including a first remapped quality score in the first group, in response to the first remapped quality score being within a first range assigned to the first group; and
   including a second remapped quality score in the second group, in response to the second remapped quality score being within a second range assigned to the second group.
12. The method of clause 1, further comprising:
   quantizing each of the plurality of remapped quality scores, to generate a plurality of quantized quality scores.
13. The method of clause 1, wherein processing the sensor data comprises:
   processing the sensor data in the base caller, to generate a sequence of base calls; and
   identifying (i) a first base call sequence in the sequence of base calls and (ii) a second base call sequence in the sequence of base calls, and further identifying that the second base call sequence has a specific base sequence pattern,
   wherein remapping the one or more of the plurality of quality scores comprises, in response to identifying that the second base call sequence has the specific base sequence pattern,
      using a first Look Up Table (LUT) to remap quality scores associated with (i) each base of the first base call sequence and (ii) a first subset of the bases of the second base call sequence, and
      using a second LUT to remap quality scores associated with a second subset of the bases of the second base call sequence.
14. The method of clause 13, wherein:
   each of a first base of the first base call sequence, a second base of the first subset of the bases of the second base call sequence, and a third base of the second subset of the bases of the second base call sequence has a quality score of Q1;
   each of the first base of the first base call sequence and the second base of the first subset of the bases of the second base call sequence is remapped, using the first LUT, to a remapped quality score of Q2;
   the third base of the second subset of the bases of the second base call sequence is remapped, using the second LUT, to a remapped quality score of Q3; and
   the remapped quality score of Q2, the remapped quality score of Q3, and the quality score of Q1 are different from each other.
15. The method of clause 13, wherein:
   the second subset of the bases of the second base call sequence includes a middle one of the bases of the second base call sequence; and
   the first subset of the bases of the second base call sequence includes all the bases of the second base call sequence, except for the middle one of the bases of the second base call sequence.
16. The method of clause 13, wherein:
   the first LUT is a general purpose LUT that is applicable to quality scores of all bases, except for a middle base of the second base call sequence; and
   the second LUT is a base sequence specific LUT specifically applicable to quality scores of the middle base of the second base call sequence.
17. The method of clause 13, wherein:
   the specific base sequence pattern comprises a homopolymer pattern or a flanked-homopolymer pattern.
18. The method of clause 13, wherein:
   the specific base sequence pattern comprises five bases, with at least a first and a last base being a G.
19. The method of clause 13, wherein:
   the specific base sequence pattern comprises at least five bases, with at least three bases of the specific base sequence pattern being a G.
20. The method of clause 13, wherein:
   the specific base sequence pattern comprises any of GGXGG, GXGGG, GGGXG, GXXGG, GGXXG, where X is any of A, C, T, or G.
21. The method of clause 13, wherein:
   the specific base sequence pattern comprises at least five bases, with at least three bases of the specific base sequence pattern associated with dark cycles within the sensor data.
22. A non-transitory computer readable storage medium impressed with computer program instructions that, when executed on a processor, implement a method comprising:
   processing sensor data for a plurality of analytes through a base caller to produce a plurality of outputs, wherein each of the plurality of output identifies a corresponding likelihood of a base incorporated in a particular one of the analytes being a corresponding one of A, C, T, or G; and
   remapping one or more of the plurality of outputs to generate a corresponding plurality of remapped outputs.
23. The non-transitory computer readable storage medium of clause 22, wherein:
   a first output of the plurality of outputs provides a first likelihood of a corresponding first analyte being one of A, C, T, or G;
   the first output is remapped to generate a first remapped output that provides a second likelihood of the corresponding first analyte being one of A, C, T, or G; and
   the first likelihood is different from the second likelihood.
24. The non-transitory computer readable storage medium of clause 23, wherein each of the first output and the first remapped output respectively express the first likelihood and the first remapped likelihood in a logarithmic scale.
25. The non-transitory computer readable storage medium of clause 23, wherein:
   the second likelihood is better aligned with an empirically determined likelihood than an alignment of the first likelihood with the empirically determined likelihood; and
   the empirically determined likelihood is a likelihood, that is determined empirically, of the corresponding first analyte being one of A, C, T, or G.
26. The non-transitory computer readable storage medium of clause 22, wherein the remapping comprises:
   identifying, from a lookup table (LUT), that a first output of the plurality of outputs is to remap to a first remapped output; and
   modifying the first output to the first remapped output, based on the LUT.
27. The non-transitory computer readable storage medium of clause 22, further comprising:
   quantizing each of the plurality of remapped outputs, to generate a plurality of quantized outputs.
28. A non-transitory computer readable storage medium impressed with computer program instructions that, when executed on a processor, implement a method comprising:
   processing sensor data for a flow cell of a sequencing machine, to predict a sequence of base calls and a plurality of quality scores associated with bases of the sequence of base calls;
   identifying (i) a first base call sequence in the sequence of base calls and (ii) a second base call sequence in the sequence of base calls, and further identifying that the second base call sequence has a specific base sequence pattern; and
   remapping the plurality of quality scores to generate a corresponding plurality of remapped quality scores, wherein the remapping comprises, in response to identifying that the second base call sequence has the specific base sequence pattern,
      using a first Look Up Table (LUT) to remap quality scores associated with (i) each base of the first base call sequence, and (ii) a first subset of the bases of the second base call sequence, and
      using a second LUT to remap quality scores associated with a second subset of the bases of the second base call sequence.
29. The non-transitory computer readable storage medium of clause 28, wherein:
   each of a first base of the first base call sequence, a second base of the first subset of the bases of the second base call sequence, and a third base of the second subset of the bases of the second base call sequence has a quality score of Q1;
   each of the first base of the first base call sequence and the second base of the first subset of the bases of the second base call sequence is remapped, using the first LUT, to a remapped quality score of Q2;
   the third base of the second subset of the bases of the second base call sequence is remapped, using the second LUT, to a remapped quality score of Q3; and
   the remapped quality score of Q2, the remapped quality score of Q3, and the quality score of Q1 are different from each other.
30. The non-transitory computer readable storage medium of clause 29, wherein:
   the second subset of the bases of the second base call sequence includes a middle one of the bases of the second base call sequence; and
   the first subset of the bases of the second base call sequence includes all the bases of the second base call sequence, except for the middle one of the bases of the second base call sequence.
31. The non-transitory computer readable storage medium of clause 28, wherein:
   the first LUT is a general purpose LUT that is applicable to quality scores of all bases, except for a middle base of the second base call sequence; and
   the second LUT is a base sequence specific LUT specifically applicable to quality scores of the middle base of the second base call sequence.
32. The non-transitory computer readable storage medium of clause 28, wherein:
   the specific base sequence pattern comprises a homopolymer pattern or a flanked-homopolymer pattern.
33. The non-transitory computer readable storage medium of clause 28, wherein:
   the specific base sequence pattern comprises five bases, with at least a first and a last base being a G.
34. The non-transitory computer readable storage medium of clause 28, wherein:
   the specific base sequence pattern comprises at least five bases, with at least three bases of the specific base sequence pattern being a G.
35. The non-transitory computer readable storage medium of clause 28, wherein:
   the specific base sequence pattern comprises any of GGXGG, GXGGG, GGGXG, GXXGG, GGXXG, where X is any of A, C, T, or G.
36. The non-transitory computer readable storage medium of clause 28, wherein:
   the specific base sequence pattern comprises at least five bases, with at least three bases of the specific base sequence pattern associated with dark cycles within the sensor data.
37. A non-transitory computer readable storage medium impressed with computer program instructions that, when executed on a processor, implement a method for generating base calls, the method including:
   processing sensor data generated by a flow cell, to generate a plurality of quality scores, each quality score of the plurality of quality scores indicative of a probability of a corresponding base to be called being a corresponding one of A, C, T, or G; and
   modifying an individual quality score to generate a corresponding individual modified quality score, thereby generating a plurality of modified quality scores.
      Clause Set 3 (Loss Penalization for specific base sequences)
1. A computer-implemented method of training a neural network model used for base calling, comprising:
   during a training phase of the neural network model of a base caller, processing sensor data in a forward pass section of the neural network model to predict base calls;
   based on the predicted base calls and ground truth base calls, generating a loss function;
   penalizing the loss function, based at least in part on the ground truth base calls indicating a specific base sequence, to generate a penalized loss function; and
   processing, in a backpropagation section of the neural network model, the penalized loss function, to adapt weights of the neural network model, thereby training the neural network model for base calling.
2. The method of clause 1, further comprising:
   identifying, from the ground truth base calls, the specific base sequence having (i) a first base and (ii) one or more second bases flanking the first base,
   wherein penalizing the loss function comprises
      penalizing (i) a first element of the loss function, which is associated with the first base, with a first penalty, and (ii) each of one or more second elements of the loss function, which are respectively associated with the one or more second bases flanking the first base, with a second penalty that is different from the first penalty.
3. The method of clause 2, further comprising:
   identifying, from the ground truth base calls, one or more third bases that are not included in the specific base sequence,
   wherein penalizing the loss function comprises
      penalizing each of one or more third elements of the loss function, which are respectively associated with the one or more third bases, with the second penalty.
4. The method of clause 2, wherein the first penalty is higher than the second penalty.
5. The method of clause 2, wherein the second penalty has a value of one.
6. The method of clause 2, wherein the first penalty has a value that is different from one.
7. The method of clause 2, wherein the first penalty has a value that is greater than one.
8. The method of clause 2, wherein the first penalty is at least twice the second penalty.
9. The method of clause 1, wherein penalizing the loss function comprises:
   multiple individual elements of the loss function with a corresponding penalty.
10. The method of clause 1, wherein penalizing the loss function comprises:
   multiple individual elements of a loss function matrix with corresponding individual elements of a penalty matrix.
11. The method of clause 1, wherein the specific base sequence comprises GGXGG, where X is any of A, C, T, or G.
12. The method of clause 1, wherein the specific base sequence comprises a homopolymer pattern or a flanked-homopolymer pattern.
13. The method of clause 1, wherein the specific base sequence comprises five bases, with at least a first and a last base being a G.
14. The method of clause 1, wherein the specific base sequence comprises at least five bases, with at least three bases of the specific base sequence pattern being a G.
15. The method of clause 1, wherein the specific base sequence comprises any of GGXGG, GXGGG, GGGXG, GXXGG, GGXXG, where X is any of A, C, T, or G.
16. The method of clause 1, wherein processing the penalized loss function comprises:
   processing the penalized loss function, to generate input gradients, wherein the input gradients are used to adapt weights of the neural network model, thereby training the neural network model for base calling.
17. A non-transitory computer readable storage medium impressed with computer program instructions that, when executed on a processor, implement a method for training a neural network model used for base calling, the method comprising:
   during a training phase of the neural network model of a base caller, processing sensor data in a forward pass section of the neural network model to predict base calls;
   based on the predicted base calls and ground truth base calls, generating a loss function;
   penalizing the loss function, based at least in part on the ground truth base calls indicating a specific base sequence, to generate a penalized loss function; and
   processing, in a backpropagation section of the neural network model, the penalized loss function, to adapt weights of the neural network model, thereby training the neural network model for base calling.
18. The non-transitory computer readable storage medium of clause 17, further comprising:
   identifying, from the ground truth base calls, the specific base sequence having (i) a first base and (ii) one or more second bases flanking the first base,
   wherein penalizing the loss function comprises
      penalizing (i) a first element of the loss function, which is associated with the first base, with a first penalty, and (ii) each of one or more second elements of the loss function, which are respectively associated with the one or more second bases flanking the first base, with a second penalty that is different from the first penalty.
19. The non-transitory computer readable storage medium of clause 18, further comprising:
   identifying, from the ground truth base calls, one or more third bases that are not included in the specific base sequence,
   wherein penalizing the loss function comprises
      penalizing each of one or more third elements of the loss function, which are respectively associated with the one or more third bases, with the second penalty.
20. The non-transitory computer readable storage medium of clause 18, wherein the first penalty is higher than the second penalty.
21. The non-transitory computer readable storage medium of clause 18, wherein the second penalty has a value of one.
22. The non-transitory computer readable storage medium of clause 21, wherein the first penalty has a value that is different from one.
23. The non-transitory computer readable storage medium of clause 21, wherein the first penalty has a value that is greater than one.
24. The non-transitory computer readable storage medium of clause 21, wherein the first penalty is at least twice the second penalty.
25. The non-transitory computer readable storage medium of clause 17, wherein penalizing the loss function comprises:
   multiple individual elements of the loss function with a corresponding penalty.
26. The non-transitory computer readable storage medium of clause 17, wherein penalizing the loss function comprises:
   multiple individual elements of a loss function matrix with corresponding individual elements of a penalty matrix.
27. The non-transitory computer readable storage medium of clause 17, wherein the specific base sequence comprises GGXGG, where X is any of A, C, T, or G.
28. The non-transitory computer readable storage medium of clause 17, wherein the specific base sequence comprises a homopolymer pattern or a flanked-homopolymer pattern.
29. The non-transitory computer readable storage medium of clause 17, wherein the specific base sequence comprises five bases, with at least a first and a last base being a G.
30. The non-transitory computer readable storage medium of clause 17, wherein the specific base sequence comprises at least five bases, with at least three bases of the specific base sequence pattern being a G.
31. The non-transitory computer readable storage medium of clause 17, wherein the specific base sequence comprises any of GGXGG, GXGGG, GGGXG, GXXGG, GGXXG, where X is any of A, C, T, or G.
32. The non-transitory computer readable storage medium of clause 17, wherein processing the penalized loss function comprises:
   processing the penalized loss function, to generate input gradients, wherein the input gradients are used to adapt weights of the neural network model, thereby training the neural network model for base calling.
33. A system for base calling, comprising:
   memory storing sensor data; and
   a base caller comprising a neural network model configured to call bases, based on the sensor data, the neural network model comprising:
      a forward pass section configured to process the sensor data, to predict base calls,
      a loss generation module configured to compare the predicted base calls and ground truth base calls, to generate a loss function,
      a loss penalization module configured to selectively penalize the loss function, to generate a penalized loss function; and
      a backpropagation section to process the penalized loss function, to facilitate adaptation of weights of the neural network model, thereby training the neural network model for base calling.

1. A computer-implemented method of generating base calls by a base caller, comprising:
receiving a plurality of sensor data from a flow cell, wherein the plurality of sensor data is within a first range;
identifying a second range, such that at least a threshold percentage of the plurality of sensor data are within the second range;
mapping at least a subset of the plurality of sensor data, that are within the second range, to a third range, thereby generating a plurality of normalized sensor data; and
processing the plurality of normalized sensor data in a base caller, to call, for the plurality of normalized sensor data, one or more corresponding bases.

2. The method of claim 1, wherein the second range is fully encompassed within the first range.

3. The method of claim 1, wherein one or more outlier sensor data within the first range are absent from the second range of sensor data.

4. The method of claim 1, wherein identifying the second range comprises:
identifying, within the first range, a low value, such that a lower threshold percentage of the plurality of sensor data have a value that is lower than the low value; and
identifying, within the first range, a high value, such that an upper threshold percentage of the plurality of sensor data have a value that is higher than the high value,
wherein the second range is defined by the low value and the high value.

5. The method of claim 4, wherein at least one of the lower threshold percentage or the upper threshold percentage is 0.5% or less.

6. The method of claim 4, wherein at least one of the lower threshold percentage or the upper threshold percentage is 1.0% or less.

7. The method of claim 4, wherein each of the lower threshold percentage and the upper threshold percentage is 0.5% or less.

8. The method of claim 4, wherein each of the lower threshold percentage and the upper threshold percentage is 1% or less.

9. The method of claim 4, further comprising:
identifying (i) a first outlier sensor data of the plurality of sensor data that is lower than the low value and (ii) a second outlier sensor data of the plurality of sensor data that is higher than the high value; and
prior to the mapping, assigning the low value to the first outlier sensor data, and assigning the high value to the second outlier sensor data, such that the first outlier sensor data and the second outlier sensor data are within the second range subsequent to the assignment.

10. The method of claim 4, further comprising:
identifying (i) a first outlier sensor data of the plurality of sensor data that is lower than the low value and (ii) a second outlier sensor data of the plurality of sensor data that is higher than the high value; and
excluding the first outlier sensor data and the second outlier sensor data from the subset of the plurality of sensor data during the mapping, for being outside the second range, such that the first outlier sensor data and the second outlier sensor data are not mapped to the third range.

11. The method of claim 1, wherein mapping at least a subset of the plurality of sensor data comprises:
mapping a first sensor data within the subset from a first value that is within the second range to a second value that is within the third range; and
mapping a second sensor data within the subset from a third value that is within the second range to a fourth value that is within the third range.

12. The method of claim 1, wherein at least a part of the second range is non-overlapping with the third range.

13. The method of claim 1, wherein individual sensor data of the plurality of sensor data comprises corresponding intensity of a corresponding section of an image generated from the flow cell.

14. The method of claim 1, further comprising:
processing the plurality of normalized sensor data in a base caller, to assign, for each base call, a first quality score indicating a probability of the called base being an A, a second quality score indicating a probability of the called base being a C, a third quality score indicating a probability of the called base being a T, and a fourth quality score indicating a probability of the called base being a G.

15. The method of claim 14, further comprising:
assigning a plurality of quality scores that includes the first quality score, the second quality score, the third quality score, and the fourth quality score; and
remapping each of at least a subset of the plurality of quality scores to a corresponding remapped quality score.

16. The method of claim 15, further comprising:
quantizing each of a plurality of remapped quality scores to a corresponding one of a plurality of quantized remapped quality score.

17. A non-transitory computer readable storage medium impressed with computer program instructions that, when executed on a processor, implement a method comprising:
receiving a plurality of intensity values from a flow cell, wherein an individual intensity value depicts a target cluster or an immediate vicinity of the target cluster of the flow cell, the target cluster populated with an unknown analyte;
identifying a second range that includes at least a threshold percentage of the plurality of intensity values;
mapping the threshold percentage of the plurality of intensity values to a third range that is different from the second range; and
subsequent to the mapping, processing the threshold percentage of the plurality of intensity values, to generate likelihoods of the unknown analyte being an A, C, T, or G.

18. The non-transitory computer readable storage medium of claim 17, wherein the second range is fully encompassed within the first range.

19. The non-transitory computer readable storage medium of claim 17, wherein one or more outlier intensity values within the first range are absent from the threshold percentage of the plurality of intensity value.

20. The non-transitory computer readable storage medium of claim 17, wherein identifying the second range comprises:
identifying, within the first range, a low value, such that a lower threshold percentage of the plurality of intensity values have a value that is lower than the low value; and
identifying, within the first range, a high value, such that an upper threshold percentage of the plurality of intensity values have a value that is higher than the high value, wherein the threshold percentage is a sum of the lower threshold percentage and the higher threshold percentage,
wherein the second range is defined by the low value and the high value.

21. The non-transitory computer readable storage medium of claim 20, wherein at least one of the lower threshold percentage or the upper threshold percentage is 0.5% or less.

22. The non-transitory computer readable storage medium of claim 20, wherein each of the lower threshold percentage and the upper threshold percentage is 1.0% or less.

23. The non-transitory computer readable storage medium of claim 20, further comprising:
identifying (i) a first outlier intensity value of the plurality of intensity values that is lower than the low value and (ii) a second outlier intensity value of the plurality of intensity values that is higher than the high value; and
prior to the mapping, assigning the low value to the first outlier intensity value, and assigning the high value to the second outlier intensity value, such that the first outlier intensity value and the second outlier intensity value are within the second range subsequent to the assignment.

24. The non-transitory computer readable storage medium of claim 20, further comprising:
identifying (i) a first outlier intensity value of the plurality of intensity values that is lower than the low value and (ii) a second outlier intensity value of the plurality of intensity values that is higher than the high value; and
excluding the first outlier intensity value and the second outlier intensity value from the subset of the plurality of intensity values during the mapping, for being outside the second range, such that the first outlier intensity value and the second outlier intensity value are not mapped to the third range.

25. The non-transitory computer readable storage medium of claim 17, wherein the mapping comprises:
mapping a first intensity value from a first value that is within the second range to a second value that is within the third range; and
mapping a second intensity value from a third value that is within the second range to a fourth value that is within the third range.

26. The non-transitory computer readable storage medium of claim 17, wherein at least a part of the second range is non-overlapping with the third range.

27. A system for base calling, comprising:
memory storing images that depict original intensity emissions of a set of analytes, the original intensity emissions generated by analytes in the set of analytes during sequencing cycles of a sequencing run;
a normalization module configured to receive the original intensity emissions and remap the original intensity emissions to generate remapped intensity emissions, such that a remapped intensity emission has a different intensity value relative to the original intensity emission; and
a base caller configured to process the remapped intensity emissions, to generate base calls for the set of analytes.
